(19) United States
(12) Patent Application Publication
(10) Pub. No.: US 2006/0062859 A1
(43) Pub. Date: Mar. 23, 2006

(54) Composition and method to optimize and customize nutritional supplement formulations by measuring genetic and metabolomic contributing factors to disease diagnosis, stratification, prognosis, metabolism, and therapeutic outcomes

(76) Inventors: Kenneth Blum, San Antonio, TX (US); Brian Meshkin, Temecula, CA (US); Bernard William Downs, Lederach, PA (US)

Correspondence Address: Brian Mashkin;Salugen, Inc., Suite 500, 4460 Le Jolla Village Drive, San Diego, CA, 92122, US

(21) Appl. No.: 11/197,980

(22) Filed: Aug. 5, 2005

Related U.S. Application Data
(60) Provisional application No. 60/599,829, filed on Aug. 5, 2004.

(51) Int. Cl. A61K 36/30; A61K 38/38; A61K 31/737; A61K 31/56

(52) U.S. Cl. 424/725; 424/765; 424/769; 435/6; 514/2; 514/54; 514/171

(57) ABSTRACT

The present invention relates to a composition and custom business model and methods to measure genetic and metabolomic contributing factors affecting disease diagnosis, stratification, and prognosis, as well as the metabolism, efficacy and/or toxicity associated with specific vitamins, minerals, herbal supplements, homeopathic ingredients, and other ingredients for the purposes of customizing a subject's nutritional supplements with custom formulations to optimize health outcomes.

### CROSS REFERENCE TO RELATED APPLICATIONS

This application claims the benefit of U.S. provisional application No. 60/599,829, filed on Aug. 5, 2004.

### FIELD OF THE INVENTION

The present invention relates to a composition and custom business model and methods to measure genetic and metabolomic contributing factors affecting disease diagnosis, stratification, and prognosis, as well as the metabolism, efficacy and/or toxicity associated with specific vitamins, minerals, herbal supplements, homeopathic ingredients, and other ingredients for the purposes of customizing a subject's nutritional supplements with custom formulations to optimize health outcomes.

### BACKGROUND OF THE INVENTION

Nutragenomics

In this patent application, we are suggesting that in this era, genes and nutrition will be the target of ongoing research. Currently, the nutraceutical world has seen only limited research in this field of nutragenomics (NGx). However, the concept of gene-based response, especially in the pharmaceutical world is growing, and billions of research dollars are being poured into the field known as pharmacogenomics (PGx). In this application, our purpose is to show how one's genome is ever important in a response to any biologically-active substance such as drugs and more importantly nutrients. As our knowledge of genomics continues to grow so will nutrigenomics in all of its facets, especially to help us understand the basis of individual differences in response to dietary patterns and targeted supplementation. Additionally, this patent application will provide ample evidence that conventional therapeutic tactics, often ignorantly based on superficial symptomatic endpoints, are inadequate and erroneous, ignoring underlying genomic requirements and gene-specific therapeutics while futily endeavoring to override the “bi-phasic” mandates of“genomic behavior” in attempts to relieve obvious symptoms (i.e. appetite suppressants for obesity; pain blockers for chronic arthritic pathologies; killing cancer cells to cure cancer; etc.) under the erroneous guise of “curing” the problem. Nutrigenomics is based on the premise that genuine optimum nutrition blunts the initiation, promotion and progression of chronic disease pathologies, satisfies normal genomic requirements, and mitigates compensatory gene-expression sequelas (such as “amplified” gene polymorphisms) that lead to a cycle of abnormal conditions/behaviors.

The recent completion of the draft sequence of the human genome and related developments has increased interest in genetics, but confusion remains among health professionals and the public at large. Inaccurate beliefs about genetics persist, including the view that in the past it had no effect on the practice of medicine and that its influence today is pervasive. We have recently entered a transition period in which specific genetic knowledge is becoming critical to the delivery of effective health care for everyone. While we do not know precisely how many genes the human genome contains, current data indicate that the human genome includes approximately 30,000 to 35,000 genes—a number that is substantially smaller than was previously thought.

If genetics has been misunderstood, genomics is even more mysterious—what exactly, is the difference? Genetics is the study of single genes and their effects. “Genomics”, a term coined only 17 years ago, is the study not just of single genes, but of the functions and interactions of all the genes in the genome. Genomics has a broader and more ambitious reach than genetics. The science of genomics rests on direct experimental access to the entire genome and applies to common conditions, such as breast cancer, colorectal cancer, human immunodeficiency, cardiovascular, Parkinson's disease and certain brain and neurological disorders such as Alzheimer's, bipolar disorder, Neurogenobolic Deficiency Syndrome (NGDS), Reward Deficiency Syndrome (RDS), and even Attention Deficit Disorder (ADHD) and related behaviors. These common disorders are also all due to the interactions of multiple genes and environmental factors.

Only about half these genes have recognizable DNA sequence patterns that suggest possible functions. Mutations known to cause disease have been identified in approximately 1000 genes. However, it is likely that nearly all genes are capable of causing disease if they are altered substantially. Whereas it was dogma that one gene makes one protein, it now appears that, through the mechanism of alternative splicing, more than 100,000 proteins can be derived from these 30,000 to 35,000 genes. Rather than DNA expression being fixed in stone, new evidence now suggests that DNA expression is a dynamic process. Forces, such as metabolomic duress caused by a variety of extraordinary factors up to an including critical disease states, can push a modification in gene expression. In addition to alternative splicing, a number of “epigenetic” phenomena, such as methylation and histone modification, can alter the effect of a gene. Furthermore, a complex array of molecular mandates allows specific genes to be “turned on” (expressed) or “turned off” in specific tissues and at specific times. Genes are distributed unevenly across the human genome. Certain chromosomes particularly 17, 19, and 22 are relatively gene dense as compared with others, such as 4, 8, 13, 18, and Y.

Interestingly, gene density varies within each chromosome, being highest in areas rich in the bases cytosine and guanine, rather than adenine and thymine. Moreover, not all genes reside on nuclear chromosomes, several dozen involved with energy metabolism are on the mitochondrial chromosome. Since ova are rich in mitochondria and sperm are not, mitochondrial DNA is usually inherited from the mother. Therefore, mitochondrial genes—and diseases due to DNA sequence variants in them—are transmitted in a matrilineal pattern that is distinctly different from the pattern of inheritance of nuclear genes.

One characteristic of the human genome with medical and social relevance is that, on average, two unrelated persons share over 99.9 percent of their DNA sequences. However, given the more than 3 billion base pairs that constitute the human genome, this also means that the DNA sequences of two unrelated humans vary at millions of bases. Since a person's genotype represents the blending of parental genotypes, we are each thus heterozygous at about 3 million bases. Many efforts are currently under way, in both the academic and commercial sectors, to catalogue these variants, commonly referred to as “single-nucleotide polymorphisms” (SNPs), and to correlate these specific genotype variations with specific genotypic variations relevant to health. Some SNP-phenotype correlations occur as a direct result of the influence of the SNP on health. More commonly, however, the SNP is merely a marker of biologic diversity that happens to correlate with health because of its proximity to the genetic factor that is actually the cause. In the case of mood there are multiple genes (polygenic inheritance) involved and thus potentially hundred's of SNPs. In general terms, the SNP and the actual genetic factor are said to be in linkage disequilibrium.

The convergence of pharmacogenetics and rapid advances in human genomics has resulted in pharmacogenomics and/or nutrigenomics, terms used here to mean influence of DNA-sequence variation on the effect of a drug and/or a natural substance or nutrient. With the completion of the Human Genome Project, and the ongoing annotation of its data, the time is rapidly approaching when the sequences of virtually all genes encoding enzymes that catalyze phase 1 and phase 11 drug metabolism will be known including genes that encode drug (nutrient)-transporters, drug (nutrient) receptors, and other drug (nutrient) targets.

It is well know that individuals respond differently to medications and certain nutraceuticals, in terms of both toxicity and treatment efficacy. Potential causes for such variability in drug (nutrient) effects include the pathogenesis and severity of the disease being treated: drug (nutrient) interactions; the individual's age, nutritional status; kidney and liver function; overall metabolic competence (especially energetic and immunological); and concomitant illnesses. Despite the potential importance of these clinical variables in determining drug/nutrient effects, it is now recognized that inherited differences in the metabolism and disposition of drugs/nutrients, and genetic variants (polymorphisms) in the targets of drug/nutrient therapy (such as receptors like the dopamine D2 receptor), can have even greater influence on the efficacy and toxicity of either medications or nutraceuticals. In a recent review written by Dervieux and Meshkin, the review authors demonstrated various proofs of principle in the field of pharmacogenetics. The review authors discussed various genes and their impact on specific drugs, as well as analyzed the pharmacoeconomic impact of these discoveries. The proofs of principle included thiopurine methyltransferase and thiopurine therapy (azathioprine and 6-mercaptopurine) for Crohns' disease and lupus, dihydropyrimidine dehydrogenase/thymidylate synthase and 5-fluorouracil therapy for chemotherapy, folate enzyme MTHFR and methotrexate therapy for rheumatoid arthritis and leukemia, UGT1a1 and irinotecan therapy for chemotherapy, and CYP450 2C9 and S-warfarin therapy for cardiovascular disease. The review authors clearly demonstrated the clinical relevance for this type of pharmacogenetic testing, and the pharmacoeconomic benefits. With advancements in the use of companion molecular diagnostic testing with pharmaceutical compounds, it is clear that such companion testing can and should be used with various nutraceutical compounds.

Clinical observations of such inherited differences in drug effects were first documented in the 1950's, exemplified by the prolonged muscle relaxation after the drug known as suxamethonium (an inhibitor of the breakdown of acetylcholine) and an inherited deficiency in the genes that encode the enzyme responsible for the breakdown of this drug as marked by plasma cholinesterase (aka acetylcholinesterase, the enzyme which breaks down acetylcholine). The second gene-based drug response was observed when researchers found that certain patients bled to death after they were treated with an anti-malarial therapy because they carried a gene variant which lowered their blood cell glucose 6-phosphate dehydrogenase activity. Such observations gave rise to the field of “pharmacogenetics” the antecedent to pharmacogenomics, the current topic. However, we now know that individual differences in response to drugs and or nutrients are not due to single gene variants but rather they are determined by the interplay of several genes encoding proteins (enzymes, receptors, transporters) involved in multiple pathways of drug/nutrient metabolism, disposition and effects. We are embarking on new era where efficacy of any substance is governed by an individual's inherited genotype to a greater degree than even other non-genetic factors. Understanding structure/function normal physiology and certain observable dysfunctions may indeed lead to promising nutrient based targets, but without the knowledge afforded by accurate DNA based prescreening (genotyping) subsequent supplementation becomes nothing more than a crap shoot. Similar to the pharmaceutical industry the nutraceutical industry can become an equal opportunity player and begin to initiate ongoing research and development by incorporating these genomic-based doctrines as described herein.

Out of the 3 million unshared DNA bases, individuals could carry gene variants (polymorphisms) that might lead to either an increase or a decrease of a certain important drug/nutrient response related proteins such as receptors, enzymes, cell cycle control, chemical messenger synthesis or catabolism (breakdown) or many other cellular events. As stared earlier, while there is a paucity of molecular studies involving genome-based response in the nutrition field (see below), a plethora of molecular studies have revealed that many genes encoding drug targets exhibit genetic polymorphism (variants), which in many cases alters their sensitivity to specific medications and/or offer specific targeted therapy.

Such examples include the following:
Asthma—Polymorphisms in Beta-adrenergic receptors (adrenalin-like) impart differential sensitivity to substances that stimulate these receptors (beta-agonists) in asthmatics.
Renal function and Blood pressure—angiotensin converting enzyme (ACE) gene polymorphisms impart differential sensitivity to inhibitors of ACE.
Cardiovascular—angiotensin 11 T1 receptor gene polymorphisms impart differential sensitivity to the substance phenylalanine and subsequent vascular reactivity.
Diabetes—polymorphisms in the sulfonylurea receptor gene imparts differential responsiveness to sulfonylurea hypoglycemic agents.
Coronary atherosclerosis—polymorphisms in the gene that controls the enzyme cholesteryl ester transfer protein impart differential efficacy of the drug pravastatin in patients with coronary disease.
Dysrythmias—Potassium channel mutations predict drug-induced dysrythmias as an adverse effect.
Drug Metabolism—Polymorphisms in cytochrome P-450 enzymes responsible for metabolizing drugs such as caffeine and codeine impart differential clearance of these and other substances. One such an enzyme is the CYP2D6.
Breast Cancer—Trasruzumab is a drug known to target a certain genetic mutation in a protein product of the HER2/neu oncogene (which is over expressed in breast cancers) and has been found compared to standard therapy to be superior in preventing metastatic breast cancer.
Diuretic therapy—There is a gene known as C825T involved with a second messenger G-protein {beta}3 whereas polymorphisms in this gene predict responsiveness to the anti-diuretic drug (used to treat hypertension), hydrochlorothiazide.
Lipid response—Genetic variation of the apolipoprotein constituents of the lipoprotein molecules (APOE gene locus) predicts plasma low-density lipoprotein cholesterol (LDL-C) concentrations. Interesting carrying one form of the APOE (E4) seems to be more responsive to dietary modification than carriers of E3 and or E2 forms of the same gene.
Nicotine patch—Variation of the CT and TT allele of the dopamine D2 receptor gene confirms a differential response to the nicotine patch. At the eight-year mark, 12% of women with the CT or TT allele of the dopamine D2 receptor gene who had received the patch had remained abstinent. Only 5% of women with the CC allele had maintained their non-smoking status. No difference based on genetics was noted in men.

Certainly we have come full circle from the “Naturalistic Era” (400 B.C. -1750 AD), to the “Chemical Analytical Era” (1750-1900) to the “Biological Era” (1900-present), to the “Cellular Era” (post 1955) and the current era of the 21st century where “genomics” is the new buzz word. Utilizing tools derived from this new science will allow us to identify and understand molecular-level interaction between nutrients and other dietary bioactives with the human genome during transcription, translation and expression, the process during which all species of proteins (glyco/lipo-proteins) encoded by the genome are synthesized and expressed. There is growing evidence that certain gene polymorphisms predict response to nutrients.

In the broadest terms, the interface between the nutritional environment and cellular/genetic processes is being referred to as “nutrigenomics”. While nutrigenomics in this sense seeks to provide a molecular genetic understanding for how common dietary chemicals (i.e. nutrition) influences health by altering the expression and/or structure of an individual's genetic makeup, the more restricted view is governed by the same principles as seen with advent of pharmacogenomics in clinical medicine, which involves DNA based—targeted response to biologically active compounds.

The tenants for nutritional genomics include in the broadest sense the following:
Common dietary substances act on the human genome
Diet can be a risk factor for a number of genetic diseases or behavioral disorders.
Diet-regulated genes are likely to play a role in the onset, incidence, progression and/or severity of chronic diseases.
Diet affects the balance between healthy and disease states and this interaction depends on an individuals genetic makeup
Excess calorie rich-nutrient deficient dietary habit-induced nutritional deficiencies, combined with increased and burdensome metabolomic load caused by those poor habits, compromises maintenance of an optimal metabolic environment, lowers the level of homeostatic equilibrium, frustrates the metabolic ability to fulfill instructions by the genetic code and increases alternate compensatory genetic assignments (expressions) with increasing dysequilibrium.
Dietary intervention based on knowledge of nutritional requirement, nutritional status, and genotype (i.e. “individualized nutrition”) can be used to prevent, mitigate, or cure chronic disease or behavioral disorders.

While there is plethora of scientific information concerned with five of the six tenets, there is paucity with regard to “individualized nutrition”.

In terms of dietary intervention based in individualized nutrition such examples of a number of gene-disease association studies have shown promise of this approach as follows:
Hypertension—The amount of circulating angiotensinogen (ANG) is associated with increased blood pressure. A SNP (polymorphism) designated AA, at nucleotide position −6 of the ANG gene, is linked with the level of blood ANG protein. Individuals with the AA genotype who eat the Dietary Approaches To Stop Hypertension (DASH) diet show reduced blood pressure, but this diet was less effective for carriers of the GG genotype.
Cardiovascular Apo—A1 gene plays a role in lipid metabolism and coronary heart disease. The A allele (variant) was associated with decreased serum HDL levels. The variant was coupled with consumption of type of fat and subsequent effect on HDL levels in both males and females carrying different genotypes.
Cancer—Methylene Tetrahydrofolate Reductase (MTHFR) is a key gene in one-carbon metabolism and, indirectly, in all methylation reactions. The C677T polymorphism of this gene, which reduces enzymatic activity, is inversely associated with occurrence of colorectal cancer and acute lymphocyte leukemia. Low intake of folate, B12, B6 and methionine was associated with increased for cancer among those with the MTHFR TT genotype.
Rheumatoid arthritis—Polymorphisms in the proinflammatory cytokine tumor necrosis factor (TNF) impart a differential response to fish oil supplementation to treat rheumatoid arthritis.
Oxidant stress and inflammation—Polymorphisms in the TNF gene impart a differential response to vitamin E to promote anti-oxidant activity and reduce inflammatory processes.
Carbohydrate metabolism—Based on polymorphisms in the gene called carbohydrate responsive element-binding protein (ChREBP), a key regulator of glucose metabolism and fat storage, Cyclic AMP and a high fat diet inhibit ChREBP and slow down glucose utilization.
Obesity—In overweight women carriers of the C polymorphisms of the Leptin receptor gene lost more weight in response to low calorie diet than the non carriers.
Central Nervous System—Extracts of Ginkgo biloba induce differential expressions of 43 cortex genes, 13 hippocampus genes, and four other genes common to both brain regions.

A Case Study: Chromium and Dopamine Genes. While there is still controversy regarding the effects of chromium salts (picolinate and nicotinate) on body composition and weight loss in general, recent work seems to support the positive change in body composition in humans. The inventors embarked on a study with chromium picolinate to test out the principles of nutrigenomics. In this study they genotyped obese subjects for the dopamine D2 receptors gene (DRD2). The subjects were assessed for scale weight and for percent body fat. The subjects were divided into matched placebo and chromium picolinate (CrP) groups. The sample was separated into two independent groups; those with either an A1/A1 or A1/A2 allele and those with only the A2/A2 allelic pattern The measures of the change in fat weight, change in body weight, the percent change in weight, and the body weight change in kilograms were all significant, whereas no significance was found for any parameter for those subjects possessing a DRD2 A1 allele. These results suggest that the dopaminergic system, specifically the density of the D2 receptors, confers a significant differential therapeutic effect of CrP in terms of weight loss and change in body fat. Moreover, the inventors propose for the first time that mixed effects now observed with CrP administration in terms of body composition, may be resolved by typing the patient via DRD2 genotyping prior to treatment with chromium salts.

There is a current interest in the relationship between toxins, diet and the role of our genes and biological response. There is emerging data showing differential response to heart disease and other medical conditions based on levels of specific toxins as well as genetics. There is some interesting data on excitotoxins and their widespread use in foods (especially in artificial sweeteners). Blaylock has reviewed the effects of such toxins like lead, aluminum, cadmium, mercury, manganese etc and biological response and the role of genes. To give just one example of an interaction between race, diet and a toxin, American Blacks tend to have a genetic vulnerability to lead due to lactose intolerance, which results in low levels of calcium in their diet. Since lead is, like calcium, a divalent cation, exposure to lead by individuals with very low calcium in their circulating blood or body stores are more likely to absorb lead. And insofar as both genetics and poverty have reinforcing effects in this vulnerability, this may have important ramifications. The inventors support the notion that the widespread effects of calcium deficiency-induced lead neurotoxcity were a significant contributor to the development of historical cultural stereotypes of black inferiority.

In terms of obesity research it is noteworthy that genetic manipulation in nutrition, metabolism may involve current standard methods for over expressing, inactivating, or manipulating genes. These molecular biology procedures can be carried out with the maintenance of the genetic information to subsequent generations (transgenic technology) or devised to exclusively transfer the genetic material to a given target organism, which cannot be transmitted to the future progeny (gene therapy). Moreover, the novel technique of RNA interference (RNAi) approach allows for the creation of new experimental models by transient ablation of gene expression by degrading specific mRNA, which can be applied to assess different biological functions and mechanisms.

DNA-Based Individualized Nutrition—Certainly, if we could get the cost of identifying a person's SNPs down to pennies rather than hundreds of dollars, we will be on the correct path to realizing nutrigenomics. Current costs of genetic tests range from $250 for prenatal tests assessing 76 diseases to $1,595 for Alzheimer's. While there are a number of companies involved in genotyping an individual's DNA, there are few that couple DNA with individualized nutrition, but no other company utilizes genetic and/or metabolomic testing to customize formulations. Other companies will recommend a host of different supplement pills, but only Salugen customizes genome specific changes to the contents of the pill.

On the other hand, tools are now available and new ones are in progress which will have relevance to the arising field of nutrigenomics. One company already involved in “individualized nutrition”, Signature Health Partners, Inc (SHP) in Ventura Calif., developed a computerized program called Nutrascan® which catalogues health priorities and screens out drug-nutrient interactions using approximately 5000 evidence—based rules which will identify individualized nutritional needs. In one scenario a person can swab their mouth for cheek cells and submit the swab to a central DNA laboratory and determine brain related neurotransmitter gene (serotonin, endorphins, GABA, dopamine, acetylcholine etc) polymorphisms. If a person carries a gene variant in the serotonin receptor (deficient) then it quite plausible to induce receptor proliferation by providing that individual a tryptophan enhancing substance like chromium and or 5-hyroxytryptophan. This may be important for adjunctive supplementation to offset some of the symptoms related to a “sweet tooth” which could ultimately result in a reduction of weight. This can then be incorporated into a program on a genome based individualized basis using the Baxter customized packeting system already utilized commercially by a number of companies. We believe that nutrigenomics is closer than ever before and will indeed be the wave of the future. We propose in this that Salugen has a unique process of analyzing this genetic information to deliver customized nutraceutical formulations by using a polymorphic, multi-variate analysis of DNA.

Salugen intends on pursuing additional DNA tests, algorithms, and nutraceutical formulations as product lines and indications related all common healthcare concerns, including but not limited to:
Alcoholism affecting 12,264,000 Americans
Drug Addiction affecting 12,500,000 Americans
Smoking Addiction affecting 46,000,000 Americans
Obesity affecting 60,000,000 Americans
Attention Deficit Hyperactivity Disorder affecting 11,200,000
Pre-Menstrual Dysphorric Disorder affecting 4,000,000 Americans

Our knowledge about the important role of glycoforms in mediating and carrying out genetic instructions is increasing dramatically. Gene-nutrition interactions especially related to genome and glycome based responses will indeed be the next cornerstone of solid scientific approaches to assist individuals in choosing dietary supplements, functional foods, and even nutritional beverages on an individualized basis. As scientists engaged in understanding the potential of drug/nutrient responses as a function of our genome, glycome and all of their ramifications including academic and commercial aspects, our future looks bright. Nutrigenomics is the key to what we have termed “nutritional gene therapy” and from its origin will spring gene and sugar mapping as the wave of the future in nutrition. The information provided in this application will serve as evidence of our conviction of this scientific opportunity.

Reward Deficiency Syndrome

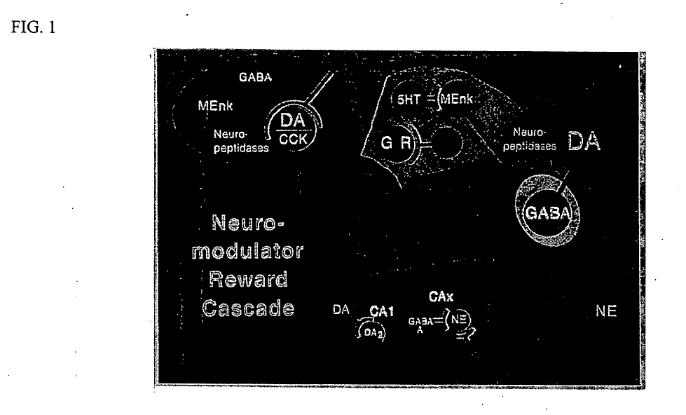

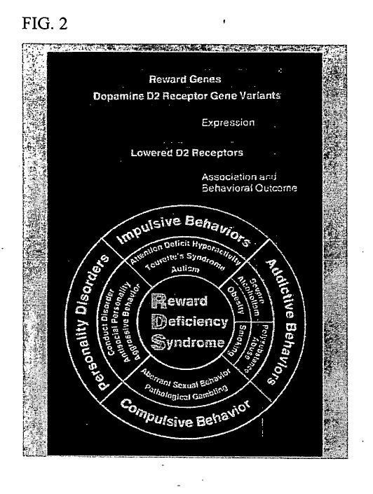

Reward Deficiency Syndrome (RDS)—In order to understand the potential role of RDS as a link to inflammation, pain, and other conditions, we provide important information as a way of background in support of the novel formula proposed in this application. Since dopamine is a major component in the mechanisms involving RDS and brain function and certain polymorphisms of the dopamine D3 receptor gene play a role in the function of prostaglandin induced transcription activity, RDS seems to be linked to flawed dopamine metabolism. The Reward Deficiency Syndrome (RDS) results from a dysfunction in the Brain Reward Cascade which directly links abnormal craving behavior with a defect in the DRD2 Dopamine Receptor Gene as well as other dopaminergic genes (D1, D3, D4, and D5), as illustrated in FIGS. 1 and 2. Dopamine is a very powerful neurotransmitter in the brain, which controls feelings of well being. This sense of well-being is produced through the interaction of dopamine and neurotransmitters such as serotonin, the opioids, and other powerful brain chemicals. Low serotonin levels are associated with depression. High levels of the opioids (the brain's opium) are associated with a sense of well-being. Kenneth Blum, Ph.D., has termed the complex interactions of these powerful neurotransmitters ultimately regulating the Dopaminergic Activity in the Reward Center of the Brain as “The Brain Reward Cascade”.

In individuals possessing an abnormality in the DRD2 Dopamine Receptor Gene, the brain lacks enough Dopamine receptor sites to use the normal amount of Dopamine in the Reward Center of the brain and thus reduces the function of Dopamine in this area of the brain. In individuals possessing the variant in the Dopamine Receptor Gene tend to be serious cocaine abusers, may have unhealthy appetites which lead to obesity or overeating or on the other extreme be anorexic with extremely low caloric intake, have levels of stress over an extended time period time period and their addictive brains lead to high generalized craving behavior. In essence they seek substances including alcohol, cocaine, nicotine, and/or glucose (substances known to cause preferential release of dopamine at the n. accumbens) to activate dopaminergic pathways as a self-healing process to offset their low D2 receptors caused by genetic antecedents known as the dopamine D2 receptor gene Taq1 A1 allele.

The overall effect is inadequate Dopaminergic Activity in the Reward Center of the Brain. This defect drives individuals to engage in activities of behavioral excess, which will increase brain Dopamine function. Consuming large quantities of alcohol or carbohydrates (carbohydrate bingeing) stimulate the brain's production of and utilization of Dopamine. So too does the intake of crack/cocaine and the abuse of nicotine. Also, it has been found that the genetic abnormality is associated with aggressive behavior, which also stimulates the brain's use of Dopamine. Such behavior exhausts nutrient availability, frustrates gene-nutrient interactions and can lead to “NeuroGenobolic Deficiency Syndrome (NGDS),” which results in further aberrant behavior (like excessive cravings and pleasure seeking) and can also produce a sort of metabolic short circuiting.

Reward Deficiency Syndrome involves a form of sensory deprivation of the brain's reward or pleasure mechanisms. Reward Deficiency Syndrome can be manifested in relatively mild or severe forms that follow as a consequence of an individual's biochemical inability to derive reward from ordinary, everyday activities. We believe that we have discovered at least one genetic aberration that leads to an alteration in the reward pathways of the brain. It is a variant form of the gene for the dopamine D2 receptor, called the A1 allele. This genetic variant also is associated with a spectrum of impulsive, compulsive, and addictive behaviors. The concept of the Reward Deficiency Syndrome unites those disorders and may explain how simple genetic anomalies give rise to complex aberrant behavior. It is our proposal that RDS is one manifestation of NGDS.

Evidence for the existence of RDS in Substance Use Disorder. In 1990, Blum and colleagues, using the Taq1 polymorphism of the dopamine D2 receptor gene locus (DRD2), for then first time reported a strong association between a virulent form of alcoholism and the minor allele A1) of the Drd2 gene in this population. Other more recent studies further support an association of the A1 allelic form of the DRD2 gene with substance abuse vulnerability and other compulsive behaviors. This association serves as the cornerstone of the biogenetic disease model and could ultimately lead us to better diagnosis and targeted treatment. A complete review of this work can be found in the Journal of Psychoactive Drugs.

This patent application will highlight the importance of a new concept, which provides a clearer understanding of impulsive, addictive, and compulsive behaviors. It is our notion that the real genesis of all behavior, whether so-called normal (socially acceptable) or abnormal (socially unacceptable) behavior, derives from an individual's genetic makeup at birth. This predisposition, due to multiple gene combinations and polymorphisms, is expressed differently based on numerous environmental elements including family, friends, educational status, economical position, environmental pollutants, and availability of psychoactive drugs including food. We believe the core of predisposition to these behaviors is a set of genes which promote a feeling of well-being via neurotransmitter interaction at the “reward site” of the brain (located in the meso-limbic system), leading to normal dopamine release. We also subscribe to the notion that at least one major gene, the dopamine D2 receptor gene, is responsible for the synthesis of dopamine D2 receptors. And further depending on the genotype (allelic form A1 versus A2), the dopamine D2 receptor gene dictates the number of these receptors at post-junctional sites.

A low number of dopamine D2 receptor suggests a hypodopaminergic function, as described by Eliot Gardner in a series of published works. When there is a paucity of dopamine receptors the person will be more prone to seek any substance (including glucose) or behavior that stimulates the dopaminergic system as a form of self-healing. In this regard we know that substances such as alcohol, cocaine, heroin, nicotine and glucose, as well as a number of behaviors like gambling and sex, preferentially release dopamine at the n. accumbens (the reward site). Understanding this preamble allows us to introduce the concept of reward deficiency syndrome into the field of addictive behavior, which will serve as a model to explain the commonality of a number of seemingly diverse addictions based on shared genetics and neurochemistry. In this regard, most recently, Qing-Shan Yan reported that ethanol, at a peak concentration within five to 10 minutes after interparenteral administration, significantly increased both extracellular dopamine and serotonin in the n. accumbens, supporting the role of these two neurotransmitters in the reinforcing properties of ethanol. Moreover, Honkanen and associates also found low basal dopamine release in alcohol accepting (AA) compared to alcohol non-accepting (ANA) rats, showing that dopamine plays a role in high alcohol preference of AA rats. One important study from Nora Volkow's group further provides support fro the role of the dopamine D2 receptor gene in alcohol intake in rats. Utilizing a cDNA construct of the dopamine D2 receptor gene implanted into the n. accumbens of rats, they found that following a four-day treatment, the dopamine D2 receptors increased to 150% above pretreatment level and alcohol drinking was reduced by 50%. After a period of 8eight days, the D2 receptor density returned to pretreatment level and so did alcohol drinking. Twenty-four days later, second injections of the same construct caused a similar increase in density with a two-fold decrease in drinking. The same group has confirmed this work in mice.

Reward Genes and The Addictive Brain—In 1990 Kenneth Blum in conjunction with Ernest P. Noble from UCLA and our colleagues, published a paper suggesting that a specific genetic anomaly was linked to alcoholism. Unfortunately it often was reported erroneously that we had found the “alcoholism gene.” Such misinterpretations are common-readers may recall accounts of an “obesity gene” or a “crime gene.” These reports imply that there is a one-to-one relationship between a gene and a specific behavior. Needless to say, there is no such thing as a specific gene for alcoholism, obesity, or criminal behavior. However, it would be naive to assert the opposite, that these complex problems of human behavior are not associated with any particular genes. Rather the issue at hand is to understand how certain genes and behaviors are connected.

In the past nine years scientists have pursued the association between certain genes and various behavioral disorders. In molecular genetics, an association refers to a statistically significant incidence of a genetic variant (an allele) among genetically unrelated individuals with a particular disease or condition compared to a control population. In the course of our work Blum and others discovered that the genetic anomaly previously found to be associated with alcoholism also is found among people with other addictive, compulsive, or impulsive disorders. The list is long and remarkable—it comprises overeating and obesity, Tourette's Syndrome, attention deficit disorder and pathological gambling. We believe these disorders are linked by a common biological substrate, a “hard-wired” system in the brain (consisting of cells and signaling molecules) that provides pleasure in the process of rewarding certain behavior. Consider how people respond positively to safety, warmth and a full stomach. If these needs are threatened or are not being met, we experience discomfort and anxiety. An inborn chemical imbalance that alters the intercellular signaling in the brain's reward process could supplant an individual's feeling of well-being with anxiety, anger or a craving for a substance that can alleviate the negative emotions. This chemical imbalance manifests itself as one or more behavioral disorders termed “Reward Deficiency Syndrome.”

This syndrome involves a form of sensory deprivation of the brain's pleasure mechanisms. It can be manifested in relatively mild or severe forms that follow as a consequence of an individual's biochemical inability to derive reward from ordinary, everyday activities. The inventors believe that we have discovered at least one genetic aberration that leads to an alteration in the reward pathways of the brain. It is a variant form of the gene for the dopamine D2 receptor, called the A1 allele (low D2 receptors), which may have been the natural prehistoric trait. This is the same genetic variant that was previously found to be associated with alcoholism as well as obesity (see below).

We look at evidence suggesting the A1 allele also is associated with a spectrum of impulsive, compulsive, and addictive behaviors, including a predisposition to overeating. The concept of the Reward Deficiency Syndrome unites these behaviors (impulsive/addictive/compulsive) and may explain how simple genetic anomalies give rise to complex aberrant behavior. Oddly enough, compared to the so called “normal” variant the A2, which occurs in approximately two-thirds of Americans having a normal compliment of D2 receptors, the A1 carriers may be predisposed to overeating, have a higher percent body fat, and have innate craving for carbohydrates.

The Biology of Reward—The pleasure and reward system in the brain was discovered by accident in 1954. The American psychologist James Olds was studying the rat brain's alerting process, when he mistakenly placed the electrodes in a part of the limbic system, a group of structures deep within the brain that generally are believed to play a role in emotions. When the brain was wired so the animal could stimulate this area by pressing a lever, Olds found that the rats would press the lever almost nonstop, as much as 5,000 times an hour. The animals would stimulate themselves to the exclusion of everything else except sleep. They would even endure tremendous pain and hardship for an opportunity to press the lever. Olds clearly had found an area in the limbic system that provided a powerful reward for these animals. Olds' research on human subjects revealed the electrical stimulation of some areas of the brain (medial hypothalamus, which is in the limbic system) produced a feeling of quasi-orgasmic sexual arousal. If certain other areas of the brain were stimulated, an individual experienced a type of light-headedness that banished negative thoughts. These discoveries demonstrated pleasure is a distinct neurological function that is linked to a complex reward and reinforcement system.

It is useful to think of the brain's reward system as a cascade in which one reaction triggers another. At the level of individual neurons, the reward cascade is catalyzed by a number of neurotransmitters. Each neurotransmitter binds to certain types of receptors and serves a specific function. The binding of the neurotransmitter to a receptor on a neuron, like a key in a lock, triggers a reaction that is part of the cascade. Disruption of these intercellular cascades results in one form or another of the Reward Deficiency Syndrome.

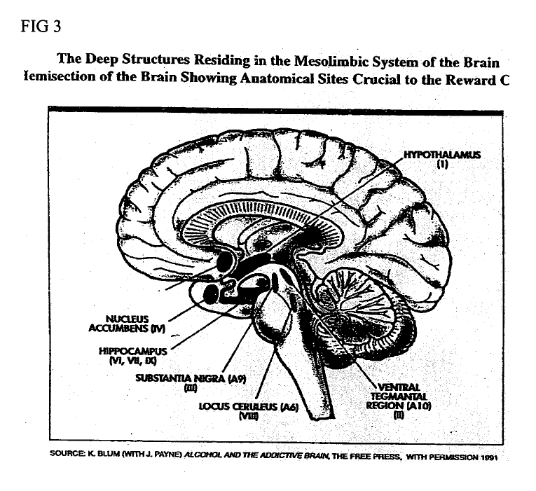

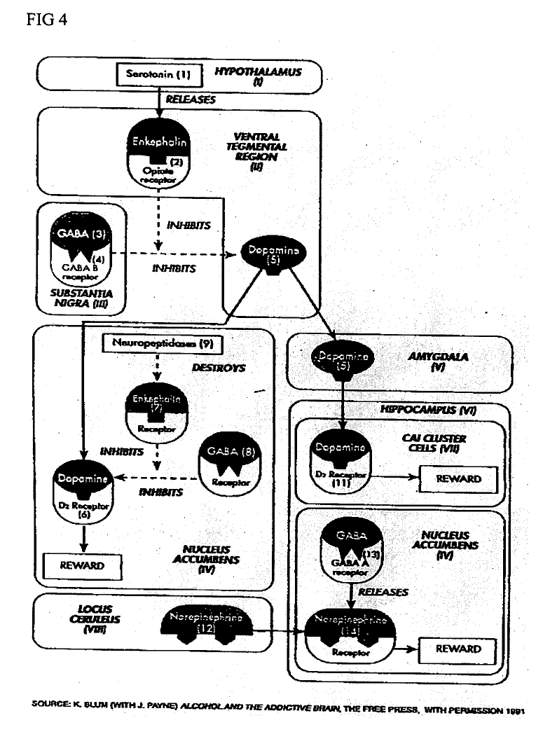

The Cascade Theory of Reward—During the past four decades, considerable attention has been devoted to the investigation of neurochemical and neuroanatomical systems underlying chemical dependency. The research on the neuropharmacological basis of dependence on alcohol, opiates, cocaine and glucose points to the involvement of common biochemical mechanisms. It appears as if a limbic-accumbens-pallidal circuit is the critical substrate for the expression of drug reward. However, while each substance of abuse appears to act on this circuit at a different step, the end result is the same, the release of dopamine the primary chemical messenger of reward at such reinforcement sites as the nucleus accumbens and the hippocampus. In a normal person, neurotransmitters (the messengers of the brain) work together in a pattern of stimulation or inhibition, the effects spreading downward from complex stimuli to complex patterns of response like a cascade, leading to feelings of well-being: the ultimate reward (Cascade Theory of Reward). Although the neurotransmitter system is too complex and still not completely understood, the main central reward areas in the human brain's meso-limbic system are illustrated and summarized in FIGS. 3 and 4.

In the reward areas the following interactions take place:
Serotonin (1) in the hypothalamus (I) indirectly activates opiate receptors (2) and causes a release of enkephalins in the ventral tegmental region A10 (II). The enkephalins inhibit the firing of GABA (3), which originates in the substantia nigra A9 region (III);
GABA's normal role, acting through GABA B receptors (4), is to inhibit and control the amount of dopamine (5) released at the ventral tegmental regions (II) for action at the nucleus accumbens (IV). When the dopamine is released in the nucleus accumbens it activates dopamine D2 receptors (6), a key reward site [there are at least five dopamine receptors, including D2]. This release also is regulated by enkephalins (7) acting through GABA (8). The supply of enkephalins is controlled by the amount of the neuropeptidases (9), which destroy them.
dopamine also may be released into the amygdala (V). From the amygdala, dopamine (10) reaches the hippocampus (IV) and the CA, cluster cells (VII) stimulates dopamine D2 receptors (11), another reward site.
an alternate pathway involves norepinephrine (12) in the locus of ceruleus A6 (VIII) whose fibers project into the hippocampus at a reward area centering around cluster cells which have not been precisely identified, but which have been designed a CAx (IX). When GABA A receptors (13) in the hippocampus are stimulated, they cause the release of norepinephrine (14) at the CAx site (See FIG. 4).

It is to be noted that the glucose receptor (GR) in the hypothalamus is intricately involved and “links” the serotinergic system with opioid peptides leading to the ultimate release of dopamine at the n. accumbens. In the “cascade theory of reward” as defined by Blum and Kozlowski, these interactions may be viewed as activities of subsystems of a larger system, taking place simultaneously or in sequence, merging in cascade fashion toward anxiety, anger, low self-esteem, or other “bad feelings” or toward craving for a substance that will make these bad feelings go away, for example sugar. Certainly, many overweight individuals also cross abuse other psychoactive substances (e.g. alcohol, cocaine, and nicotine). Alcohol activates the norepinephrine fibers of the mesolimbic circuitry through a cascade of events, including the interaction of serotonin, opioid peptides, and dopamine. In a more direct fashion, through the subsequent formation of the neuroamine condensation products TIQs, alcohol may either interact with opioid receptors or directly with dopaminergic systems.

In the cascade theory of carbohydrate bingeing, genetic anomalies, long-continued stress, or long-term abuse of sugar can lead to a self-sustaining pattern of abnormal craving behavior in both animals and humans. Animal model support for the cascade theory can be derived from a series of experiments carried out by T. K. Li et al., upon their substance-preferring (P) [seek carbohydrates, alcohol, opiates, etc.] and nonpreferring (NP) rat lines. They found that P rats have the following neurochemical profile:
lower serotonin neurons in the hypothalamus;
higher levels of enkephalin in the hypothalamus (due to a lower release);
more GABA neurons in the nucleus accumbens;
reduced dopamine supply at the nucleus accumbens;
reduced densities of dopamine D2 receptors in the meso-limbic areas.

This suggests a four-part cascade sequence leading to a reduction of net dopamine release in a key reward area. This was further confirmed when McBride et al. found that administering substances which increase the serotonin supply at the synapse, or by stimulating dopamine D2 receptors directly, craving behavior could be reduced. Specifically, D2 receptor agonists reduce alcohol intake in high alcohol preferring rats whereas D2 dopamine receptor antagonists increase alcohol drinking in these inbred animals.

Inhibitors of Enkephalinase(s) and Craving Behavior—As stated earlier, although it is known that opiates and/or opioids reportedly increase food intake in animals and humans, some papers suggest the opposite-suppression of food intake, especially when one considers macro selection of food sources (i.e., sugar/carbohydrates). Moreover, Broekkamp et al. reported that infusion of enkephalin into the ventral tegmental A10 area of the brain induces a short-term latency behavioral stimulant effect reminiscent of effects produced by stimulation of the meso-limbic dopamine pathway; this effect is blocked by pretreatment of the opiate receptor antagonist naloxone. This takes on importance in terms of feeding behavior, as feeding has been shown to increase dopamine levels in various brain structures such as the posterior hypothalamus, the nucleus accumbens, and the amygdala.

It is well known that dopamine in sufficient concentration can inhibit food intake. Gilman and Lichtingfeld proposed as an appropriate therapeutic for carbohydrate bingeing (i.e., bulimia) a selective D2 agonist such as bromocriprtine [or natural released dopamine], providing D2 occupancy. In this regard, using a push-pull cannula technique, Chesselet et al., were able to induce dopamine release in the “brain reward center” after local application of enkephalin, which suggests regulation by delta receptor stimulation. Indeed Kelotorphan (an inhibitor of the opioid peptide degrading enzyme) may protect against possible CCK-8 degradation by brain peptidases. This important satiety neuropeptide is co-localized with dopamine in the nucleus accumbens, and there is a close interaction between CCK-8, dopamine, and endogenous opioid peptides (like enkephalins). The opioid peptides are involved not only in macro-nutrient intake, but have been implicated in substance seeking, as well as brain self-stimulation behavior. In essence, there are a substantial number of animal experiments which support not only the “Brain Reward Cascade” but the subsequent sequela induced by a defected reward cascade leading to a number of addictive, compulsive and impulsive behaviors—defined as the “Reward Deficiency Syndrome”.

In this regard, Blum et al. reversed alcohol-seeking behavior in genetically preferring C57B1/6J mice with the chronic administration of an enkephalinase inhibitor. In other work by George et al., they concluded that a relative lack of enkephalin peptides trans-synaptically, possibly resulting from enhanced enkephalin degradation, might contribute to increased alcohol consumption in C57B1/6J mice. Moreover, others showed that intracranial self-stimulation by rats was reduced by nucleus accumbens microinjections of kelatrophan, a potent enkephalinase inhibitor.

Brain Hypodopaminergic Function and The Self-Healing Process—Since deficits have been found in neurotransmitter functions underlying craving behavior, and since these deficits may be alleviated by facilitated dopamine release consequent to the use of drugs, nicotine, alcohol, and food, the studies mentioned above indicate enkephalinase inhibition may similarly compensate for neurotransmitter imbalance (i.e., opioids, thereby attenuating craving behavior). In an attempt to understand that carbohydrate craving is a subset of generalized craving behavior (“Reward Deficiency Syndrome”), due to hypodopaminergic function (an impaired “reward cascade”), scientists believe individuals self-heal through biochemical (illicit or non-illicit) attempts to alleviate the low dopaminergic brain activity via drug-receptor activation (alcohol, heroin, cocaine, and glucose). It is conjectured this will substitute for the lack of reward and yield a temporary sense of well-being. In order to help explain this so called self-healing process, it is germane that the reinforcing properties of many drugs of abuse may be mediated through activation of common neurochemical pathways, particularly with regard to the meso-limbic dopamine system. In this regard, glucose, opiates, nicotine, cocaine, tetrahydrocannibinol (THC), and ethanol have been shown to directly or indirectly enhance release or block re-uptake of dopamine in at least one of the primary terminal sites for the limbic dopamine neurons, the nucleus accumbens.

A number of studies of genetically bred animal models support the D2 dopamine receptor involvement in substance-seeking behavior due to lower D2 receptor sites in preferring compared to non-preferring animals. One inference from these observations is that ethanol intake, as well as the self-administration of other substances (i.e., glucose), might be altered by manipulation of dopamine receptors. Of interest, Gardener observed further confirmation of the “Reward Deficiency Syndrome” in generalized substance-behavior involving slow dopamine release in the nucleus accumbens in polysubstance seeking Lewis animals.

Reward Deficiency Syndrome: Human Studies—Human support for the Reward Deficiency Syndrome can be derived from a series of clinical trials with macronutrients (precursor amino acid loading technique and enkephalinase inhibition) indicating:
Reduced alcohol and cocaine craving
Reduced stress rates
Reduction of leaving treatment against medical advice (AMA)
Facilitated recovery
Reduced relapse rates
Reduction in carbohydrate bingeing
Loss of body weight
Prevention of weight regain
Reduction of glucose craving
Enhancement of insulin sensitivity (Reversal of Metabolic Syndrome X)
Reduction of cholesterol
Enhancement of memory and focus
Enhanced compliance with narcotic antagonists.

There are a number of studies using precursor amino-acids and enkephalinase inhibition that have been shown to affect various aspects of RDS [see Table 1 below].

TABLE 1
Summary of Completed Clinical Studies with Nutraceutical Supplementation (A Literature Review)

| Drug | | | | | | |
|---|---|---|---|---|---|---|
| Abused | | | | | | |
| or Dys- | Supplement | No. of | No. of | Study | | |
| function | Used | Patients | Days | Type | Significant Results | Publication |
| Alcohol | SAAVE | 22 | 28 | TO | 100% decrease in BUD scores. Detoxification measures: | Blum K, Trachtenberg MC, Ramsey |
| | | | | IP | reduction in benzodiazepine requirement, reduction in | J. Improvement of inpatient treatment |
| | | | | | withdrawal tremors after 72 hours, reduction in | of the alcoholic as a function of |
| | | | | | depression | neuronutrient restoration: a pilot |
| | | | | | | study. Int J Addiction. 1988; |
| | | | | | | 23: 991-98. |
| | | | | | | Blum K, Trachtenberg MC. |
| | | | | | | Neurogenic deficits caused by |
| | | | | | | alcoholism: restoration by SAAVE. |
| | | | | | | Journal of Psychoactive Drugs. 1988; |
| | | | | | | 20: 297. |
| Alcohol | SAAVE | 62 | 21 | DBPC | Reduction in psychosocial stress reduction as measured | Blum et al. Enkephalinase inhibition |
| plus | | | | IP | by SCL, reduced BESS score, improved physical score, | and precursor amino acid loading |
| Poly- | | | | | six-fold decrease in likelihood of leaving AMA after five | improves inpatient treatment of |
| drugs | | | | | days. | alcoholics and poly-drug abusers: a |
| | | | | | | double-blind placebo-controlled study |
| | | | | | | of the neuronutrient intervention |
| | | | | | | adjunct SAAVE. Alcohol. 1989; |
| | | | | | | 5: 481. |
| Cocaine | Tropamine | 54 | 30 | TO | Drug hunger significantly reduced in patients taking | Blum et al. Reduction of both drug |
| | | | | IP | SAAVE as compared to controls: 4.2 percent AMA rate | hunger and withdrawal against advice |
| | | | | | for patients on Tropamine versus 28 percent for patients | rate of cocaine abusers in a 30 day |
| | | | | | on SAAVE and 37 percent for controls. | inpatient treatment program with the |
| | | | | | | neuronutrient tropamine. Curr Ther |
| | | | | | | Res. 1988; 43: 1204. |
| Alcohol | SAAVE and | 60 | 379 | TO | At end of one year over 50 percent of the alcoholic DUI | Brown et al. Neurodynamics of |
| and | Tropamine | | | CP | offenders not using SAAVE dropped out of the program | relapse prevention: a neuronutrient |
| Cocaine | | | | | while less than 15 percent of those using SAAVE | approach to outpatient DUI offenders. |
| | | | | | dropped out. For the cocaine abusers over 90 percent | J. Psychiatric Drugs. 1990; 22: 173. |
| | | | | | of the Non-Tropamaine group dropped out, but less than | |
| | | | | | 25 percent of the patients in the control group. | |
| Over- | PCAL 103 | 27 | 90 | TO | The PCAL 103 group lost an average of 27 pounds in 90 | Blum et al. Neuronutrient effects on |
| Eating | | | | OP | days compared with an average loss of 10 pounds for | weight loss on carbohydrate bingeing |
| | | | | | the control group. Only 18.2 percent of the PCAL 103 | in a bariatric setting. Curr Ther Res. |
| | | | | | patient group relapsed compared to 82 percent of the | 1990; 48: 2a17. |
| | | | | | patients in the control group. | |
| Over- | PCAL 103 | 247 | 730 | PCOT | After two years. craving and binge eating were reduced | Blum K, Cull JG, Chen JHT, |
| Eating | | | | OP | one-third in group of patients on PCAL 103, as | Garcia-Swan S, Holder JM, Wood R, |
| | | | | | compared to the control patients. PCAL 103 group | et al. Clinical relevance of PhenCal |
| | | | | | regained 14.7 pounds of their lost weight compared with | in maintaining weight loss in an |
| | | | | | 41.7 percent weight regained in control patients. | open-label, controlled 2-year study. |
| | | | | | | Curr Ther Res. 1997; 58: 745-63. |
| Over- | Chromium | 40 | 112 | RDBPC | 21 percent increase (p < 0.001) in resting metabolic rate | Kaats FE et al. The short-term |
| Eating | Picolinate | | | CP | (RMR), no change in lean body mass (LBM), RMR: LBM | therapeutic effect of treating obesity |
| | (CP) and L- | | | | increased 25 percent (p < 0.001). Body fat decreased | with a plan of improved nutrition and |
| | Camitine | | | | approximately 1.5 lbs./week, and reduction in serum | moderate caloric restriction. |
| | | | | | cholesterol while increasing RMR with no loss of LBM | Curr Ther Res. 1992; 51: 261. |
| Over- | Chromium | 32 | 180 | DBPC | After six months the CrP group had an increase in lean | Bahadori B, Habersack S, Schneider |
| Eating | Picolinate | | | OP | body mass and avoided non-fat related weight loss. | H, Wascher TC. Topiak H. Treatment |
| | | | | | Difference between groups was significant at p < 0.001. | with chromium picolinate improves |
| | | | | | | lean body mass in patients following |
| | | | | | | weight reduction. |
| | | | | | | Federation Am Soc Exp Bio 1995. |
| Over- | Chromium | 154 | 72 | RDBPC | 200 and 400 mcg of CrP brought about significant | Kaats FE, Blum K, Fisher JA, |
| Eating | Picolinate | | | OP | changes in Body Mass composition indicies when | Aldeman JA. Effects of chromium |
| | | | | | compared with placebo | picolinate supplementation on body |
| | | | | | | mass composition: a randomized, |
| | | | | | | double-blind, placebo-controlled |
| | | | | | | study. Curr Ther Res. 1996; |
| | | | | | | 57: 747-56 |
| Over- | Chromium | 122 | 90 | RDBPC | After controlling for differences in caloric expenditure | Kaats FE, Blum K, Pullin D, Keith |
| Eating | Picolinate | | | OP | and caloric intake as compared with the placebo group, | SC, Wood R. A randomized |
| | | | | | 400 mcg CrP group lost significantly more weight | double-masked placebo-controlled |
| | | | | | (p < 0.001) and body fat (p < 0.004), had a greater | study of the effects of chromium |
| | | | | | reduction in body fat (p < 0.001), significantly improve | picolinate supplementation on body |
| | | | | | body composition (p < 0.004). | composition: a replication of previous |
| | | | | | | study. Curr Ther Res. 1998; |
| | | | | | | 59: 379-88. |
| Over- | Chromium | 122 | 90 | RDBPC | Measures of changes in fat weight, change in body | Blum K, Kaats G, Eisenbery A, |
| Eating | Picolinate | | | OP | weight, percent change in weight, and body weight | Sherman M, Davis K, Comings DE, |
| | | | | | changes in kgms were all significant in A2/A2 group, | Cull JG. Chen THJ, Wood R, |
| | | | | | and non-significant in A1/A2 and A1/A1 carriers. | Bucci L, Wise JA, Braverman ER, |
| | | | | | | and Pullin D. Chromium Picolinate |
| | | | | | | Induces Changes in Body |
| | | | | | | Composition as a Function of the |
| | | | | | | Taq1 Dopamine D2 Receptor A1 |
| | | | | | | Alleles. Submitted to International |
| | | | | | | J. Eat. Dis. |
| Over- | Chromium | 43 | 63 | ROTPC | CrP supplementation resulted in significant weight gain, | Grant KE, Chandler RM, Castle AL, |
| Eating | Picolinate | | | OP | while exercise training combined with CrP | Ivy JL. Chromium and exercise |
| | and | | | | supplementation resulted in significant weight loss and | training: effect on obese women. |
| | Chromium | | | | lowered insulin response to an oral glucose load. | J Am Sports Med 1997; 29(8): |
| | Picolinate | | | | Concluded high levels of CrP supplementation are | 992-8. |
| | comparison | | | | contraindicated for weight loss, in young obese women. | |
| | | | | | Moreover, results suggested that exercise combined | |
| | | | | | with CrP supplementation may be more beneficial than | |
| | | | | | exercise training alone for modification of certain CAD or | |
| | | | | | NIDDM risk factors | |
| Healthy | Tropagen | 15 | 30 | DBPC | Non-drug subjects with Tropagen performed better on | Defrance JJ, Hymel C, Trachtenberg |
| Volun- | | | | OP | computer memory and performance tasks as measured | MC et al. Enhancement of attention |
| teers | | | | | with P300 wave evoked potential. Changes in P300 | processing by Kantrol in healthy |
| | | | | | wave evoked potential result in better focusing ADHD | humans: A pilot study. Clin |
| | | | | | patients | Electroencephalgr. 1997; 28: 68-75. |

Abbreviations used:
BUD—building up to drink;
AMA—withdrawal against medical advice;
OP—outpatient;
MMPI—Minnesota Multiphasic personality inventory;
DB—double-blind;
IP—inpatient;
SCL—skin conductance level;
BESS—behavioral, emotional, social, spiritual;
DBPC—double-blind placebo-controlled;
DUI—driving under the influence;
R—randomized;
TO—open trial

Most recently, research by Ortiz and associates at Yale University School of Medicine and the University of Connecticut Health Services Center supported the notion of dopamine as the “final common pathway” for a number of diverse drugs of abuse such as cocaine, morphine, and alcohol (as well as glucose). This support demonstrates that chronic treatment with cocaine, morphine, or alcohol similarly result in several biochemical adaptations in the meso-limbic dopamine system, which may “underlie prominent changes in the structural and functional properties of the neuronal pathway” related to the above. The brain reward cascade schematic (illustrated in FIG. 4), since then, became the blueprint for the search for “reward genes”. We propose that the Reward Deficiency Syndrome gives rise to a wide range of disorders that can be classified as impulsive-addictive-compulsive diseases. Impulsive diseases include attention deficit disorder and Tourette's Disorder. Addictive diseases include substance-seeking behavior involving alcohol, drugs, nicotine, and most importantly food. Compulsive diseases include pathological gambling and excessive sexual activity. In terms of personality disorders it includes conduct disorder, oppositional defiant disorder, antisocial personality disorder, schizoid/avoidant behavior, violent aggressive behaviors (See FIG. 1).

Reward Genes—Historical background—In the late 1980's Blum was inspired by a Jane England (1987) paper reporting the association of a variant found on chromosome 11 at the tyrosine hydroxylase loci in bi-polar affective disorder among the Amish. This molecular genetic observation coupled with the then current research on the inheritance of alcoholism provided the impetus for Blum, and associates to investigate potential genetic differences between alcoholics and nonalcoholics. They suspected that one of the differences was the activity of chemical signaling molecules in the brain. Over the course of two years they compared eight genetic markers associated with various neurotransmitters and metabolic enzymes (including serotonin, endogenous opioids, GABA, transferrin, acetylcholine, and alcohol and aldehyde dehydrogenases). In each instance they failed to find a direct association between the genetic markers and alcoholism. Finally, as we stated above, Blum, Noble and others began to study the gene which controls the laying down of dopamine D2 receptors; the dopamine D2 receptor gene. They found a very significant association between the Dopamine D2 receptor gene and severe alcoholism. In their original study over 70 percent of the alcoholics had cirrhosis of the liver, a disease suggestive of severe and chronic alcoholism. Quickly following this first study published in the Journal of the American Medical Association (JAMA), a number of other flawed studies were negative. The negative studies failed to adequately assess controls to eliminate alcoholism, drug abuse, and other related “reward behaviors” including carbohydrate bingeing and used less severe alcoholics. In this regard, Drs. Katherine Neiswanger and Shirley Hill of the University of Pittsburgh (funded by the National Institutes of Alcoholism and Alcohol Abuse) found a strong association of the D2 A1 allele and alcoholism. Hill suggested failures reported in the literature were due to poor assessment of controls. Their suggestion significantly bolsters the appropriate use of “super” controls to more accurately assess a true phenotype. This is especially important when studying complex behavioral diseases. The same researchers found evidence for linkage between the dopamine D2 receptor gene and severe alcoholism, early onset, physical dependence symptoms, and Antisocial Personality Disorder.

Joint Health

While often referred to as if it were a single disease, arthritis is actually an umbrella term used for a group of more than 100 medical conditions that collectively affect nearly 70 million adults and 300,000 children in America alone. While the most common form of arthritis—osteoarthritis (OA)—is most prevalent in people over 60, arthritis in its various forms can start as early as infancy. Some forms affect people in their young-adult years as they are beginning careers and families and still others start during the peak career and child-rearing years.

The common thread among these 100-plus conditions is that they all affect the musculoskeletal system and specifically the joints—where two or more bones meet. Arthritis-related joint problems include pain, stiffness, inflammation and damage to joint cartilage (the tough, smooth tissue that covers the ends of the bones, enabling them to glide against one another) and surrounding structures. Such damage can lead to joint weakness, instability and visible deformities that, depending on the location of joint involvement, can interfere with the most basic daily tasks such as walking, climbing stairs, using a computer keyboard, cutting your food or brushing your teeth.

For many people with arthritis, joint involvement is not the extent of the problem. Many forms of arthritis are classified as systemic, meaning they can affect the whole body. In these diseases, arthritis can cause damage to virtually any bodily organ or system, including the heart, lungs, kidneys, blood vessels and skin. Arthritis-related conditions primarily affect the muscles and the bones.

According to the Arthritis Foundation, arthritis and related conditions are a major cause of disability in the United States, costing the U.S. economy more than $124 billion per year in medical care and indirect expenses such as lost wages and production—and costing millions of individuals their health, their physical abilities and, in many cases, their independence. And unless something changes, the picture is going to get worse. As the population ages, the number of people with arthritis is growing.

Number of Americans with arthritis or chronic joint symptoms (Source: Arthritis Foundation, www.arthritis.org, searched on Jun. 16, 2005):
1985-35 million
1990-37.9 million
1998-nearly 43 million (1 in 6 people)
2005-66 million (nearly 1 in 3 adults)

Based upon the Arthritis Foundation web site, arthritis is one of the most prevalent chronic health problems and the nation's leading cause of disability among Americans over age 15. Arthritis is second only to heart disease as a cause of work disability. Arthritis limits everyday activities such as walking, dressing and bathing for more than 7 million Americans. Arthritis results in 39 million physician visits and more than a half million hospitalizations. Costs to the U.S. economy totals more than $86.2 billion annually. Arthritis affects people in all age groups including nearly 300,000 children. Baby boomers are now at prime risk. More than half those affected are under age 65. Half of those Americans with arthritis don't think anything can be done to help them. Arthritis refers to more than 100 different diseases that affect areas in or around joints. Arthritis strikes women (41 million) more often than men (almost 29 million).

The disease also can affect other parts of the body. Arthritis causes pain, loss of movement and sometimes swelling. Some types of arthritis that impact joint health are:
Osteoarthritis, a degenerative joint disease in which the cartilage that covers the ends of bones in the joint deteriorates, causing pain and loss of movement as bone begins to rub against bone. It is the most prevalent form of arthritis.
Rheumatoid arthritis (RA), an autoimmune disease in which the joint lining becomes inflamed as part of the body's immune system activity. Rheumatoid arthritis is one of the most serious and disabling types, affecting mostly women. New insights indicate that in RA, impaired galactosylation alters the requisite three dimensional configurations of glycoprotein structures, including certain immune factors, such as IGg and possibly type II collagen, producing the loss of self recognition and identity. This loss of self-identification alters recognition and response signaling during immune surveillance, inciting attack on the body's own joint collagen. Alterations of glycosylation and galactosyl structures are hallmark characteristics of RA. (Other autoimmune disorders have also been associated with faulty glycosylation.)
Gout, which affects mostly men, is usually the result of a defect in body chemistry. This painful condition most often attacks small joints, especially the big toe. Fortunately, gout almost always can be completely controlled with medication and changes in diet.
Ankylosing spondylitis (AS), a type of arthritis that affects the spine. As a result of inflammation, the bones of the spine grow together. Ankylosing spondylitis is also associated with a misrecognition event in which HLA-B57 is mistakenly identified as an unwelcome foreign antigen, which incites unremitting attacks that result in the characteristic damage of AS.
Juvenile arthritis, a general term for all types of arthritis that occur in children. Children may develop juvenile rheumatoid arthritis or childhood forms of lupus, ankylosing spondylitis or other types of arthritis.
Systemic lupus erythematosus (lupus) is a serious disorder that can inflame and damage joints and other connective tissues throughout the body.
Scleroderma is a disease of the body's connective tissue that causes a thickening and hardening of the skin.
Fibromyalgia, is where widespread pain affects the muscles and attachments to the bone. It affects mostly women.

There are more than 100 types of arthritis that affect joint health, and related conditions affecting approximately 70 million Americans today. A complete listing includes Achilles tendonitis, Achondroplasia, Acromegalic arthropathy, Adhesive capsulitis, Adult onset Still's disease, Ankylosing spondylitis, Anserine bursitis, Avascular necrosis, Behcet's syndrome, Bicipital tendonitis, Blount's disease, Brucellar spondylitis, Bursitis, Calcaneal bursitis, Calcium pyrophosphate dihydrate (CPPD), Crystal deposition disease, Caplan's syndrome, Carpal tunnel syndrome, Chondrocalcinosis, Chondromalacia patellae, Chronic synovitis, Chronic recurrent multifocal osteomyelitis, Churg-Strauss syndrome, Cogan's syndrome, Corticosteroid-induced osteoporosis, Costostemal syndrome, CREST syndrome, Cryoglobulinemia, Degenerative joint disease, Dermatomyositis, Diabetic finger sclerosis, Diffuse idiopathic skeletal hyperostosis (DISH), Discitis, Discoid lupus erythematosus, Drug-induced lupus, Duchenne's muscular dystrophy, Dupuytren's contracture, Ehlers-Danlos syndrome, Enteropathic arthritis, Epicondylitis, Erosive inflammatory osteoarthritis, Exercise-induced compartment syndrome, Fabry's disease, Familial Mediterranean fever, Farber's lipogranulomatosis, Felty's syndrome, Fibromyalgia, Fifth's disease, Flat feet, Foreign body synovitis, Freiberg's disease, Fungal arthritis, Gaucher's disease, Giant cell arteritis, Gonococcal arthritis, Goodpasture's syndrome, Gout, Granulomatous arteritis, Hemarthrosis, hemochromatosis, Henoch-Schonlein purpura, Hepatitis B surface antigen disease, Hip dysplasia, Hurler syndrome, Hypermobility syndrome, Hypersensitivity vasculitis, Hypertrophic osteoarthropathy, Immune complex disease, Impingement syndrome, Jaccoud's arthropathy, Juvenile ankylosing spondylitis, Juvenile dermatomyositis, Juvenile rheumatoid arthritis, Kawasaki disease, Kienbock's disease, Legg-Calve-Perthes disease, Lesch-Nyhan syndrome, Linear scleroderma, Lipoid dermatoarthritis, Lofgren's syndrome, Lyme disease, Malignant synovioma, Marfan's syndrome, Medial plica syndrome, Metastatic carcinomatous arthritis, Mixed connective tissue disease (MCTD), Mixed cryoglobulinemia, Mucopolysaccharidosis, Multicentric reticulohistiocytosis, Multiple epiphyseal dysplasia, Mycoplasmal arthritis, Myofascial pain syndrome, Neonatal lupus, Neuropathic arthropathy, Nodular panniculitis, Ochronosis, Olecranon bursitis, Osgood-Schlatter's disease, Osteoarthritis, Osteochondromatosis, Osteogenesis imperfecta, Osteomalacia, Osteomyelitis, Osteonecrosis, Osteoporosis, Overlap syndrome, Pachydermoperiostosis Paget's disease of bone, Palindromic rheumatism, Patellofemoral pain syndrome, Pellegrini-Stieda syndrome, Pigmented villonodular synovitis, Piriformis syndrome, Plantar fasciitis, Polyarteritis nodosa, Polymyalgia rheumatica, Polymyositis, Popliteal cysts, Posterior tibial tendonitis, Pott's disease, Prepatellar bursitis, Prosthetic joint infection, Pseudoxanthoma elasticum, Psoriatic arthritis, Raynaud's phenomenon, Reactive arthritis/Reiter's syndrome, Reflex sympathetic dystrophy syndrome, Relapsing polychondritis, Retrocalcaneal bursitis, Rheumatic fever, Rheumatoid arthritis, Rheumatoid vasculitis, Rotator cuff tendonitis, Sacroiliitis, Salmonella osteomyelitis, Sarcoidosis, Saturnine gout, Scheuermann's osteochondritis, Scleroderma, Septic arthritis, Seronegative arthritis, Shigella arthritis, Shoulder-hand syndrome, Sickle cell arthropathy, Sjogren's syndrome, Slipped capital femoral epiphysis, Spinal stenosis, Spondylolysis, Staphylococcus arthritis, Stickler syndrome, Subacute cutaneous lupus, Sweet's syndrome, Sydenham's chorea, Syphilitic arthritis, Systemic lupus erythematosus (SLE), Takayasu's arteritis, Tarsal tunnel syndrome, Tennis elbow, Tietse's syndrome, Transient osteoporosis, Traumatic arthritis, Trochanteric bursitis, Tuberculosis arthritis, Arthritis of Ulcerative colitis, Undifferentiated connective tissue syndrome (UCTS), Urticarial vasculitis, Viral arthritis, Wegener's granulomatosis, Whipple's disease, Wilson's disease, and Yersinial arthritis.

Dopamine and Pain: Brain Reward Cascade—The principle ascending pathways for pain (e.g. spinothalamic tract) originate mainly in the dorsal horn of the spinal cord and medulla wherein second order neurons receive synaptic input from primary afferent neurons that supply nociceptors in tissue. The second order neurons of origin are within layer I as well as deep layers (IV-VI) of the dorsal horn. Second order neurons of origin of pain-related pathways are mainly wide dynamic range (WDR) neurons or nociceptive-specific (NS) neurons and these two types of neurons process both exteroceptive and interoceptive information associated with pain. Our cutaneous nociceptive system clearly serves as an exteroreceptive role in signaling potentially dangerous stimuli impinging upon our bodies, so that we can respond appropriately, depending upon the situational context. Our interoreceptive nociceptive system signals tissues disorders (e.g. rheumatoid) that are essentially inescapable, and calls for responses more obviously in the homeostatic domain.

Pharmacological aspects of pain control—Opioids such as morphine and heroin and psychostimulant drugs such as amphetamine and cocaine are effective pharmacological tools against chronic pain. Interestingly, amphetamine and related drugs relieve cancer pain and sometimes administered as an adjuvant analgesic in the clinical situation because they potentiate opioid analgesia and counter opioid-related sedation and cognitive disturbances. In support of these clinical findings, studies have shown that, in rats, psychostimulants potentiate the analgesic effect of morphine in an animal model of persistent pain. There is increasing evidence that sites rostral to the brainstem play a critical role in the analgesic effects of opioid and psychostimulant drugs. It is well known that opioids can inhibit pain by acting at spinal sites and at sites in the brainstem where they modulate activity in descending brain stem pathways projecting to the spinal cord. A primary site of action is the periacqueductal gray of the brain stem, where stimulation of opioid receptors activates through direct projections, serotonin-containing cells in the nucleus raphe magnus. In turn, the latter cells activate neurons that project, via the dorsolateral funiculus, to the dorsal horns of the spinal cord where they inhibit cells that transmit information about noxious painful stimulation from the periphery to supraspinal sites. The brainstem—descending pain-suppression system, however, plays a more important role in the suppression of brief, rapidly rising, transient, and well-localized (i.e. phasic) pain than it does in the suppression of injury—produced persistent (i.e. tonic) and inescapable pain. However, several lines of evidence suggest that the inhibition of the tonic pain requires the activation of neural systems in addition to those requires the activation of neural systems in addition to those required for inhibition of phasic pain.

Mesolimbic dopamine in the suppression of tonic pain—There is little information to date concerning the identity of the endogenous pain systems that serve to inhibit tonic pain. The suppression of tonic pain involves systems in addition to those known to suppress phasic pain, and that these systems appear to involve forebrain sites, rostral to the brainstem. A clue to this problem is that both opioids and psychostimulants reduce tonic pain and increase transmission in mesocorticolimbic dopamine neurons known to be activated by natural rewards such as food and sex. These neurons arise from dopamine cell bodies that lie in the ventral tegmental area (VTA) and project to various forebrain sites such as the nucleus accumbens (Nacc), amygdala, and prefrontal cortex. Opioids cause the release of dopamine from these neurons through their indirect activation (see reward cascade), whereas psychostimulant drugs such as amphetamine and cocaine increase dopamine extracellularly by decreasing reuptake and/or inducing release. Moreover, opioids and psychostimulants have both rewarding effects and analgesic effects in the clinical setting, suggesting that reward and analgesia might share common neural substrates. Morgan and Franklin found that dopamine-depleting 6-hydroxydopamine lesions of the ventral midbrain, which contains the cell bodies of the neurons that give rise to ascending forebrain projections, block the analgesic effects of systemic morphine and amphetamine in the formalin, but not the tail-flick test. Their findings provided the first evidence that mesolimbic dopamine neurons play a role in the suppression of tonic, but not phasic pain. In recent studies, Taylor et al. found that while the D1—selective agonist SKF 38393 was without effect at a dose of 0.5 nmol/side, the D2—selective agonist quinpirole dose dependency (0.05-5.onmol/side, bilateral) inhibited the persistent phase of formalin-induced nociception. This was blocked by pre-administration of a selective S2-dopaminergic antagonist raclopride. These results indicate dopamine agonists that activate D2 receptors in the Nacc, inhibit inflammatory pain.

Dopamine D2 receptors and chronic pain—Plastic changes in synaptic neurotransmission in the brain are thought to play a role in chronic pain. Animal studies suggest that striatal and cortical dopaminergic systems participate in pain transmission or modulation. Dopamine D2 receptors have been reported to mediate the inhibitory role of dopamine in animal models for persistent pain. Hagelberg et al., showed that in healthy volunteers that high D2 receptor availability in the putamen is associated with low cold pain threshold and a high pain modulation capacity induced by conditioning stimulation. Furthermore, decreased [18F] FDOPA uptake and increased D2 receptor availability have been demonstrated in the putamen in a chronic orofacial pain state, the burning mouth syndrome. Moreover, it was found that the increase in D2 receptor availability in the left putamen and the decrease in D1/D2 ratio imply that alterations in the striatal dopaminergic system as evaluated by PET may be involved in chronic orafacial pain conditions. In essence, we hypothesize that low or hypodopaminergic function in the brain may predispose individuals to low pain tolerance. Current research would support this concept and thus carriers of the D2 Taq A1 allele as observed in RDS behaviors may be good candidates for nutrients designed to enhance dopamine release in the brain.

Stress in America—The effects of excessive stress in modern life leads to chronic states of fatigue-related depression. This is an unfortunate fact yet true that about 80% of all illness can be traced back to stress and depression. The American Academy of Family Physicians suggests that about ⅔ of office visits relate to stress.

The importance here is to understand that it is our position that indeed in an obese individual or a carbohydrate binger the subject is definitely in a stressful condition and therefore there is increased neuronal firing. There are numerous examples in the literature to support this contention. Furthermore, if an obese individual has the DRD2A1 variant, numerous studies have shown that resultant low dopamine D2 receptors caused an inability to cope with stress in the family and as an individual. In this regard, it is known that stress could even reduce D2 receptor mRNA message in the substantia nigra, the lateral part of the VTA, basal ganglia especially in the “reward site” the nucleus accumbens. This work supports the concept that forebrain dopamine systems are involved in mediating the behavioral effects of chronic mild stress. It further supports the view that in obese subjects (with chronic mild to moderate stress) with a compromised number of D2 receptor sites and reduced mRNA message, the firing frequency of a catecholaminergic neuron is enhanced and would be quite receptive to l-tyrosine supplementation as proposed in the formula. Moreover, it is also known that neuronal depletion of dopamine could also induce an independent end-product inhibitory state for TOH, which will also respond to l-tyrosine supplementation. With a slow release formula, there is constant dopamine release because of the effect of enhanced opioidergic activity via d-phenylalanine on substantia nigra GABA neurons.

Stress and dopamine: Implications for the pathophysiology of chronic widespread pain—The relationship between stress, endorphins and hypothalamic-pituitary-adrenal (HPA) axis is well known. Certainly in the world of addiction stress plays a critical role in both the acquisition and relapse. It is known that certain genetic and environmental elements play significant roles in drug dependency and dysregulation of brain reward pathways. In fact, dopamine D2 receptor polymorphisms have been associated with stress coping mechanisms and posttraumatic stress disorder. Interestingly, either stress can induce a painful condition or it can exacerbate the pain. Exposure to stress also activates dopamine transmission in mesocorticolimbic dopamine neurons and this effect appears to involve opioid mechanisms in the VTA. More specifically, intra-VTA infusions of the opioid receptor antagonist, Naltrexone, prevent the stress-induced activation of dopamine metabolism in the NAcc and prefrontal cortex, and exposure to stress causes the release of met-enkephalin into the VTA. These findings combined with those indicating that exposure to stress can inhibit tonic pain and that intra-VTA morphine induces analgesia in the formalin test, suggest that the endogenous release of opioids in the VTA might be a mechanism underlying the stress-induced inhibition of tonic pain. This has been supported by the finding that intra-VTA infusions of the opioid receptor antagonist, Naltrexone, stress-induced analgesia in the formalin test. In addition, it has been proposed that release of the tachykinin neuropeptide, substance P (SP), in the VTA might play a similar role in the stress-induced suppression of tonic pain. In this regard, Altier and Stewart have also found that activation of midbrain dopamine neurons by SP did indeed inhibit tonic pain in the formalin test. The current data suggests that exposure to stress induces analgesia by causing a release of SP in the VTA, which in turn activates mesocorticolimbic dopamine neurons. Finally, opioids, amphetamine, and SP all share the ability to increase dopamine release in the NAcc. Moreover, opioids administered systemically or into the VTA augment dopamine metabolism and extracellular levels of dopamine in the NAcc.

With that background it becomes increasingly clear that tonic pain maybe attenuated by dopamine D2 activation. It follows then that in this application we embrace as one inventive embodiment a natural method to cause a preferential release of dopamine in mesocorticolimbic pathways. In this regard, support of an attenuation of stress has be found with a variant of the Synaptamine complex proposed herein in a double-blind placebo controlled study. We propose herein that unless there is a way of increasing endogenous opioids, which in turn inhibit GABA causing dopamine release in the NAcc, simple neurotransmitter precursors will not be as effective in reducing tonic pain.

Fibromyalgia—One example of how stress and dopamine may interact involves fibromyalgia (FM), which has been called a “stress-related disorder” due to the onset and exacerbation of symptoms in the context of stressful events. The cardinal feature of FM is pain, the experience of which involves both afferent and efferent processes. While exposure to acute stress is known to produce stress-induced analgesia, the induction of which depends on of dopamine containing neurons within the NAcc, rat studies have demonstrated that prolonged exposure to stress eliminates this response, resulting instead in a state of stress-induced hyperalgesia. Chronic stress has been shown to result in the attenuation of dopaminergic activity within the NAcc and is therefore proposed to contribute to the development of stress-related hyperalgesia.

Interestingly, in FM patients clinical studies have suggested a disruption of dopaminergic function, including but not limited to decreased dopamine metabolites in cerebrospinal fluid. A variety of stressors result in the release of dopamine within the NAcc, including acute psychological stress a cornerstone symptom of FM. Thus, a vicious cycle occurs whereby stress from the pain further exacerbates the release of dopamine, which in turn results in a hyperalgesia state. Hyperalgesia to both thermal and chemical stimulants persists up to 9 days after stress exposure in rats. Moreover, other neurotransmitters are also involved as well. The selective 5-HT reuptake inhibitors clomipramine and fluextine, as well as the 5-HT reuptake precursor tryptophan, blocks development of hyperalgesia, suggesting that repeated stress produces a long-lasting increase in pain sensitivity. In fact, whereas there is a disruption of both serotinergic and dopaminergic function that occurs within the NAcc following chronic stress, the impact on dopamine outlasts that of 5-HT. In this regard there are three possibilities which have been proposed: (1) there is regulatory interaction between 5-HT and Dopamine during stress-induced analgesia; (2) a disruption of this interaction contributes to the inception of stress-induced hyperalgesia; and (3) dopaminergic dysfunction, which outlasts that of 5-HT, may be responsible for the persistent expression of stress-induced hyperalgesia after serotinergic function has been normalized. This phenomenon may explain why strategies aimed at boosting serotinergic function only on patients with chronic widespread pain have met with limited success insofar as analgesia is concerned. Thus since FM is a stress-related disorder, one would predict that strategies aimed at boosting dopaminergic function within the mesolimbic pathway would have superior efficacy. While no one has attempted combining therapies in term of multiple pharmacogenomic targets, and the outcome of such an attempt is unknown, on this we are proposing that natural manipulation of the reward signaling and circuitry could become very commercially viable. Breaking of this cycle with a stress reducing substance, such as the proposed Synaptamine with a genetically customized formulation of nutritional supplements, is clearly warranted.

There are many nutritional supplements that have been studied and/or are being used to improve joint health and reduce the signs and symptoms associated with these forms of arthritis. The following list put together by Arthritis Today, a publication of the Arthritis Foundation, outlines each of these ingredients, their respective sources, their forms and typical “one size fits all” dosage, anticipated efficacy, studies, and potential risks.

BLACK CURRANT OIL, BLACK CURRANT SEED OIL, Ribes nigrum
Where It Comes From: Black currant seed oil obtained from seeds of the black currant. Do not confuse with black currant berry. Black currant seed oil contains 6 percent to 19 percent gamma-linolenic acid (GLA).
Forms and Dosage: Capsules; typical dosage ranges from 500 mg to 1,000 mg black currant oil daily. Look for capsules with concentrations of GLA varying from 0.2 grams to 0.3 grams.
Extras: May increase immune response in elderly.

BORAGE OIL, BORAGE SEED OIL, Borago officinalis
Where It Comes From: Oil from the seeds of the Borage plant. Borage seed oil contains about 20 percent to 26 percent of the essential fatty acid GLA.
Forms and Dosage: Capsules; 1,300 mg (for oil) daily. Look for capsules with concentrations of GLA varying from 0.2 grams to 0.3 grams.

BOVINE CARTILAGE
Where It Comes From: Ground cartilage of cow, usually from the trachea or windpipe.
Forms and Dosage: Capsule, powder and cream; no typical dosage.
What It's Supposed to Do: Anti-inflammatory agent, believed to ease the symptoms of osteoarthritis (OA) and rheumatoid arthritis (RA). Topical bovine cartilage may treat psoriasis and aid in wound healing.
What We Know: Like shark cartilage, researchers think bovine cartilage may support resynthesis of cartilage.
Studies: Some anti-inflammatory effects shown in a few in vitro and animal studies. No good, well-controlled clinical studies support arthritis claim.
What to Watch For: Can cause diarrhea, nausea, swelling, local redness and itching. May enhance the anticoagulant effect of certain drugs like nonsteroidal anti-inflammatory drugs (NSAIDs). Potential for contamination from diseased animal parts related to mad cow disease, however no cases have been reported.

BROMELAIN Pineapple, Ananas comosus
Where It Comes From: Enzyme found in pineapple stems and juice that breaks down protein.
Forms and Dosage: Tablets; 80 mg to 320 mg, two or three times per day for eight to 10 days or as needed for more than 10 days.
What It's Supposed to Do: Decrease pain and swelling of RA and OA, increase mobility.
What We Know: Some evidence that enzymes like bromelain, which break down protein, have pain-relieving and anti-inflammatory effects comparable to NSAIDs. Likely safe.
Studies: In one human study, a bromelain supplement containing enzymes rutin and trypsin relieved pain and improved function in 73 people with knee OA. The effect was similar to taking an NSAID. No research shows bromelain alone is effective for people with arthritis.
What to Watch For: Bromelain can cause stomach upset and diarrhea. Avoid if allergic to pineapples. Can increase the effect of blood-thinning medicine.
Extras: May help reduce swelling after surgery or injury. May have potential to help burn wounds and accelerate healing.

CAT'S CLAW, Uncaria tomentosa
Where It Comes From: Dried root bark of a woody vine that grows in the Amazon rain forests in Peru.
Forms and Dosage: Capsules, tablets and tea bags; dosage varies.
What It's Supposed to Do: Believed to have anti-inflammatory properties. Used for treating knee OA.
What We Know: Although widely used for a variety of illnesses from diverticulitis and ulcers to cancer and AIDS, there is little clinical research to support these claims.
Studies: The most promising work comes from a study in which 100 grams daily of freeze-dried cat's claw reduced knee pain in people with OA.
What to Watch For: Headache, dizziness and vomiting. Cat's claw can lower blood pressure, so don't use if taking an antihypertensive medication. Cat's claw might adversely affect people with autoimmune disorders. Avoid using if you have RA, lupus, multiple sclerosis or other autoimmune disorders.

CETYL MYRISTOLEATE, CETYL-M, CMO, Cis-9-cetylmyristoleate
Where It Comes From: Waxy, fat-like substance that comes from mice, believed to protect them against developing arthritis.
Forms and Dosage: Capsules and creams; no typical dose.
What It's Supposed to Do: Lubricate joints, regulate immune system, act as an anti-inflammatory, ease symptoms of RA, OA, fibromyalgia, Sjöegren's syndrome, lupus and ankylosing spondylitis.
What We Know: Cetyl-m prevents mice from getting arthritis, but there's no evidence it works in humans or other animals.
Studies: No well-designed clinical studies to support claims.

CHONDROITIN SULFATE
Where It Comes From: Chondroitin is a component of human cartilage, bone and tendon. In supplements, chondroitin sulfate usually comes from bovine trachea or pork byproducts.
Forms and Dosage: Capsules, tablets and powder; 1,200 mg daily in two doses.
What It's Supposed to Do: Reduce pain and inflammation, improve joint function and slow disease progression.
What We Know: Chondroitin is believed to enhance the shock-absorbing properties of collagen and block enzymes that break down cartilage. Currently there is no proof that it can reverse cartilage loss. It generally takes two to four months to work completely.
Studies: Many studies using chondroitin have been small and scientifically flawed. However, two large studies that evaluated data from about a dozen studies showed significant improvement in pain and inflammation and improved joint function. Some people taking chondroitin are able to decrease NSAID dosage.
What to Watch For: Diarrhea, constipation and abdominal pain. Some chondroitin tablets may contain high levels of manganese, which may be problematic with long-term use. Because chondroitin is made from bovine products, there is the remote possibility of contamination associated with mad cow disease. Chondroitin taken with blood-thinning medication like NSAIDs may increase your risk of bleeding.
In the Works: There is no convincing evidence that glucosamine and chondroitin together are more effective than each one individually or alone. To find out, the National Institutes of Health (NIH) recently commissioned a large, multi-centered study to be completed by 2005.

COLLAGEN HYDROLYSATE, COLLAGEN, GELATIN, GELATINE, GELATIN HYDROLYSATE, HYDROLYZED [Denatured] COLLAGEN
Where It Comes From: The main protein that makes up human and animal cartilage. In supplements, it can come from pig, cow, ox, chicken or sheep.
Forms and Dosage: Capsules, tablets or powder; 10 grams daily.
What It's Supposed to Do: Relieve pain, inflammation, swelling and stiffness of RA, OA, juvenile rheumatoid arthritis (JRA) and gout. Repair cartilage.
What We Know: Scientific evidence of the effectiveness of collagen hydrolysate is controversial. Most work supporting collagen hydrolysate has been done in Germany.
Studies: A recent review of the role of collagen hydrolysate in bone and joint disease found no differences in pain between pharmaceutical-grade collagen hydrolysate (PCH) and a placebo. PCH did, however, appear to improve the collagen absorption of cartilage when combined with calcitonin, a hormone that stops the bone from losing calcium.
What to Watch For: Stomach upset and nausea. Do not take chicken collagen if allergic to chicken or egg. In bovine sources, there is the remote potential for contamination associated with mad cow disease. Note: Do not confuse with Undenatured Type II Chicken collagen.

DEVIL'S CLAW, DEVIL'S CLAW ROOT, GRAPPLE PLANT OR WOOD SPIDER Harpagophytum procumbens
Where It Comes From: A traditional herbal plant used in South Africa and Namibia.
Forms and Dosage: Capsules, powdered root and tea; six 435 mg capsules daily; 1 gram to 4.5 grams daily of powdered root or tea.
What It's Supposed to Do: Relieve pain and inflammation. Act as a digestive aid and appetite stimulant.
What We Know: The active ingredient in devil's claw is harpagoside, which appears to reduce pain and inflammation in joints. Some studies suggest stomach acid may counteract benefits. To avoid this problem, take the supplement between meals when stomach acid is at its lowest.
Studies: One recent clinical human study showed devil's claw relieved OA pain in the knee and hip, especially when used with NSAIDs. People taking devil's claw may be able to decrease use of NSAIDs.
What to Watch For: Do not take if you have ulcers or are taking an antacid. It can affect heart rate and may interfere with cardiac, blood-thinning and diabetes medication. Do not take if you have gallstones. May cause diarrhea.

DHEA—Dehydroepiandrosterone
Where It Comes From: An androgen steroid hormone naturally produced in the body.
Forms and Dosage: Capsule and tablets available both as prescription and non-prescription products; typically 200 mg for lupus. Do not take without a prescription.
What It's Supposed to Do: Control lupus.
What We Know: DHEA levels have been found to be particularly low in people with RA and lupus. May regulate immune system. Has been submitted to FDA for approval for the treatment of lupus.
Studies: DHEA used in conjunction with conventional lupus treatment may reduce disease activity and flares. In studies it also allowed doctors to lower women's glucocorticoid dosages. DHEA also appeared to counteract bone loss caused by medication and increase bone density. Long-term safety, overall effectiveness and appropriate dosages have not been established.
What to Watch For: Acne, stomach upset, abdominal pain and high blood pressure. Decreases HDL levels and may cause facial hair growth, voice deepening and changes in menstrual pattern. Can increase insulin resistance or sensitivity for people with diabetes. Can affect liver, if you have liver disease.

DMSO Dimethyl Sulfoxide—See MSM
Where It Comes From: A colorless sulfur-containing organic liquid used as an industrial solvent and a by-product of paint thinner and anti-freeze.
Forms and Dosage: Cream, gel, injection, liquid or solution; topically, 70 percent to 90 percent DMSO solution; internally, take only if prescribed by a physician.
What It's Supposed to Do: Relieve pain and inflammation, improve joint mobility in OA, RA, JRA and scleroderma, and manage amyloidosis (excessive build-up of protein in organs as seen in RA).
What We Know: Topically, appears to be an anti-inflammatory.
Studies: Controlled studies as a topical application for DMSO and OA have yielded conflicting results. Few human studies.
What to Watch For: Headache, dizziness, drowsiness, nausea, vomiting, diarrhea, constipation and anorexia. Topical DMSO can also cause skin irritation and dermatitis. Do not use if you are have diabetes, asthma, or liver, kidney or heart conditions.
Extras: Never take industrial-grade DMSO.

EVENING PRIMROSE OIL, EVENING PRIMROSE OR PRIMROSE—Oenothera biennis and other Oenothera species.
Where It Comes From: The small seeds of a native American wildflower, containing 2 percent to 16 percent gamma-linolenic acid (GLA).
Forms and Dosage: Capsules; generally five 500 mg capsules per day. For RA, 540 mg daily to 2.8 grams daily. Look for capsules with concentrations of GLA ranging from 2 grams to 3 grams.

FEVERFEW Tanacetum parthenium
Where It Comes From: Fresh or dried leaves of the feverfew plant, which grows in Europe.
Forms and Dosage: Capsules, tablets, whole fresh leaves or dried leaves; capsules or tablets of the freeze-dried leaf, 50 mg to 125 mg daily standardized to 0.6 percent to 0.7 percent of the active ingredient parthenolide; two to three leaves taken with food daily.
What It's Supposed to Do: Decrease pain and inflammation.
What We Know: A few animal studies show feverfew may reduce inflammation, but results have been mixed. The few human studies have shown no benefit to arthritis.
What to Watch For: Stomach upset, nausea, diarrhea, flatulence and vomiting. Do not take if you are allergic to plants in the daisy family including ragweed, marigolds and others. Chewing feverfew can cause mouth sores, swelling of the mouth, tongue and lips, and loss of taste. Do not take for longer than four months.

FISH OIL
Where It Comes From: Oil from cold-water fish such as mackerel, salmon, herring, tuna, halibut and cod liver.
Forms and Dosage: Fish, capsules or pills; one serving of fish two to three times a week; capsules or pills, typically 3 grams EPA/DHA—the active ingredient in omega-3 fatty acids—daily.
What It's Supposed to Do: Fight inflammation, lessen fatigue and reduce morning stiffness. Treat RA, lupus, psoriasis, depression and Raynaud's syndrome. DHA is important for brain function and may inhibit RA development.
What We Know: Fish oil is made up of omega-3 fatty acids.
Studies: More than 20 clinical trials have shown fish oil supplements, alone and in combination with conventional drugs, to be effective in treating RA symptoms. Polymorphisms in the proinflammatory cytokine tumor necrosis factor (TNF) impart a differential response to fish oil supplementation to treat rheumatoid arthritis.
What to Watch For: Nausea, diarrhea, heartburn and nosebleeds. Acts as a blood thinner, so do not take if already taking anticoagulant medication. Fish oil can suppress the immune system, increase blood sugar and lower blood pressure.
Extras: Fish oil lowers blood triglyceride (fats that circulate in the blood stream) levels, protecting against heart disease and reducing high blood pressure. One study from Israel found EPA may enhance antidepressant medication in people with recurrent depression. Also helps to regulate heart rhythm, so it protects against sudden cardiac death.

FLAXSEED and FLAXSEED OIL, FLAX OIL or LINSEED OIL Linum usitatissimum
Where It Comes From: Seed of the flax plant, primarily composed of omega-3 fatty acids and lignans (beneficial plant compounds, similar to fiber).
Forms and Dosage: Whole seeds, ground meal or flour, capsules or oil; whole seeds must be ground into meal or flour; ground meal or flour, 30 grams (1 ounce) daily; capsules, available in 1,000 mg to 1,300 mg, no typical dosage; oil, 1 to 3 tablespoons daily.
What It's Supposed to Do: Ease symptoms of RA, lupus and Raynaud's syndrome. Lubricate joints and lessen stiffness and joint pain.
What We Know: Flaxseed is high in alpha-linolenic acid, a type of omega-3 fatty acid that can be converted to EPA and DHA (the active ingredients in fish oil).
Studies: No good studies showing flaxseed affects RA, but omega-3 fatty acids are known to be anti-inflammatory agents.
What to Watch For: Flaxseed is a natural laxative, so increase consumption slowly and drink plenty of liquids. Fiber in flaxseed can impair absorption of some medications. Flaxseed acts as blood thinner, so beware when taking blood thinners, aspirin or other NSAIDs. Should be avoided by women with hormone-sensitive breast and uterine cancer, and by people with high cholesterol.
Extras: Flaxseed oil should not be heated. It spoils quickly, so it must be kept in a dark bottle and refrigerated. Flaxseed lowers total and LDL (bad) cholesterol, reduces risk of heart disease and cancer, and is a good source of fiber.

GINGER—Zingiber officinale
Where It Comes From: The dried or fresh root of the ginger plant.
Forms and Dosage: Powder, extract, tincture, spice and oils; 225 mg twice daily.
What It's Supposed to Do: Decrease joint pain and reduce inflammation in people with OA and RA. Protect stomach from ulcers and damaging gastrointestinal effects of NSAIDs.
What We Know: Ginger contains active ingredients that may have analgesic and anti-inflammatory properties. Ginger reduces nausea and vomiting.
Studies: A double-blind clinical study using highly purified ginger extract found ginger reduced knee OA pain. Dosages used in the study were 225 mg twice daily.
What to Watch For: Heartburn, diarrhea and stomach discomfort. Can interfere with medications for blood pressure, blood-thinning, heart, diabetes or antacid. Do not use if you have gallstones.

GINGKO—Ginkgo biloba
Where It Comes From: Leaf of the gingko tree, native to China.
Forms and Dosage: Liquid, tablet and capsule; typically 120 mg to 240 mg daily. Choose supplements standardized to 6 percent terpene lactones and 24 percent flavone glycosides, the active ingredients in gingko.
What It's Supposed to Do: Increase blood flow and circulation in Raynaud's syndrome.
Studies: No research on Raynaud's syndrome, but clinical evidence shows that gingko helps circulation.
What to Watch For: Stomach upset, dizziness or headaches. Do not take if you are taking blood-thinning medication like aspirin, have epilepsy or experience seizures, have diabetes, or are scheduled for surgery.
Extras: Gingko leaf may boost memory and cognition.

GINSENG—American Ginseng: Panax quinquefolius, Asian Ginseng: Panax ginseng, Siberian Ginseng: Eleutherococcus senticosus
Where It Comes From: The root of the ginseng plant, native to North America and Asia.
Forms and Dosage: Capsules, tablets, tea, tincture powder or tincture tonic; capsules or tablets made from dried root; typically 200 mg to 600 mg daily. As a tea, use 1 teaspoon of fresh grated ginseng steeped in one cup of water one to three times a day for three weeks.
What It's Supposed to Do: Increase ability to deal with stress, alleviate fatigue, “boost” immune system, increase stamina and improve cognitive function.
What We Know: Little known about what it does or how it might work. Effectiveness is unknown.
Studies: One study done in Mexico on hundreds of people showed those taking 40 mg of ginseng and a multivitamin appeared to have improved quality of life and sense of well-being. However, a recent evaluation of 16 well-controlled clinical trials found no evidence that ginseng was effective in treating any condition.
What to Watch For: Do not take if you have a heart condition, diabetes, a hormone-sensitive condition, schizophrenia, high or low blood pressure, have had an organ transplant, are pregnant or are taking medication that has blood-thinning effects, medication that suppresses the immune system, or MAO inhibitors. Can amplify effects of glucocorticoids such as prednisone, cause insomnia and act as a stimulant.
Extras: Asian ginseng is the most-studied supplement. Approximately six million Americans use it regularly.

GLA (GAMMA-LINOLENIC ACID)
Where It Comes From: A type of omega-6 fatty acid found in evening primrose oil, black currant oil and borage oil.
Forms and Dosage: Capsules or oil; 2 grams to 3 grams daily.
What It's Supposed to Do: Lessen joint pain, stiffness and swelling associated with RA. Ease symptoms of Raynaud's syndrome and Sjöegren's syndrome.
What We Know: Several studies show GLA (in all three oils) is an effective treatment for reducing inflammation in RA with few side effects. GLA only works if taken orally; there is no evidence that these oils applied topically are effective. It also may regulate the immune system.
Studies: One of the most promising studies was a placebo-controlled trial of 56 patients with active RA who received 2.8 grams of GLA for six months. Participants showed significant improvements related to joint pain, stiffness and grip strength. GLA doses at this level were concluded to be safe and effective for RA.
What to Watch For: GLA is a blood thinner so may increase risk of bleeding if taken with NSAIDs, anticoagulant medications or blood-thinning supplements. Orally, evening primrose oil can cause indigestion, nausea, soft stools and headache. Borage seed oil may exacerbate liver disease.
Extras: GLA increases effectiveness of drugs used to treat breast cancer. Increases HDL (the “good” cholesterol) and decreases fat levels in blood stream. Helps treat nerve problems due to diabetes.

GLUCOSAMINE—Glucosamine sulfate, glucosamine hydrochloride, N-acetyl glucosamine
Where It Comes From: Major component of joint cartilage. Supplements are derived from the shells of shellfish such as shrimp, lobster and crab.
Forms and Dosage: Capsules, tablets, liquid or powder (to be mixed into a drink); 1,500 mg per day for all forms. Because not as much is known about the glucosamine hydrochloride and N-acetyl glucosamine, some researchers encourage people interested in trying this product to use glucosamine sulfate.
What It's Supposed to Do: Slow deterioration of cartilage, relieve OA pain and improve joint mobility.
What We Know: Glucosamine provides the natural building blocks for growth, repair and maintenance of cartilage. Like chondroitin, it helps cartilage absorb water and keeps the joint lubricated. Effects may be similar to NSAIDs for easing OA symptoms but may take twice as long as conventional drugs to work.
Studies: Studies on glucosamine are promising. A review of two studies, each of which analyzed more than a dozen glucosamine studies, found this supplement to significantly and consistently improve pain and joint function, as well as or better than conventional drug therapy (NSAIDs). One recent long-term study conducted in Belgium over three years showed patients with mild to moderate knee OA taking 1,500 mg of glucosamine had 20 percent to 25 percent less pain and disability than those taking the placebo. Researchers also found glucosamine slowed, if not stopped, the progression of the disease and reduced cartilage loss. Glucosamine has been studied only in people with knee or hip OA. More studies are needed for long-term safety and effectiveness to be established.
What to Watch For: Mild stomach upset, nausea, heartburn, diarrhea, constipation and increased blood glucose, cholesterol, triglyceride and blood pressure levels. Don't use if you are allergic to shellfish.
Extras: To determine effectiveness, NIH is conducting a large long-term study on glucosamine, chondroitin and a combination of the two in people with knee OA. Results are expected in 2005.

GOTU KOLA, GOTU COLA, BRAHMI, BRAHMA-BUTI, INDIAN PENNYWORT Centella asiatica
Where It Comes From: Plant that grows in India, Japan, China, South Africa and Indonesia.
Forms and Dosage: Capsules, tablets, tincture, creams, ointments, dried leaves or tea; standardized extracts in capsule or liquid form, 60 mg to 120 mg per day; tablets, 600 mg three times per day; tea, three cups daily, with one quarter to one half teaspoon dried herb to one cup boiling water.
What It's Supposed to Do: Reduce fatigue, decrease pain, improve circulation and ease symptoms for RA, psoriasis, ankylosing spondylitis and lupus.
What We Know: Gotu kola contains plant compounds that may have anti-inflammatory and analgesic effects, but evidence is weak. Preliminary evidence in animal studies suggests these compounds may prevent and treat gastrointestinal ulcers. Topically, gotu kola appears to help psoriasis.
Studies: No clinical human studies to support claims.
What to Watch For: Stomach upset, nausea, drowsiness, sensitivity to sunlight and increased blood pressure, glucose and cholesterol levels. May interfere with hypertension and diabetes medications. Topically, can cause allergic dermatitis and burning sensation. Do not take for more than six weeks unless your doctor advises otherwise.

GRAPESEED, GRAPESEED OIL, GRAPESEED EXTRACT Vitis vinifera
Where It Comes From: Seeds of grapes from a woody vine native to Asia minor.
Forms and Dosage: Tablets or capsules; 75 mg to 300 mg grapeseed extract daily for three weeks followed by a maintenance dosage of 40 mg to 80 mg per day.
What It's Supposed to Do: Fight inflammation, relieve symptoms of chronic fatigue and fibromyalgia, and improve circulation.
What We Know: A potent antioxidant, grapeseed oil contains flavonoids (beneficial plant compounds), essential fatty acids and tocopherol (vitamin E). Effectiveness has not been proven.
Studies: No human clinical studies on grapeseed and arthritis. Preliminary evidence suggests grapeseed may strengthen connective tissue.
What to Watch For: Grapeseed increases risk of bleeding, so do not use if taking medication or supplements with blood-thinning effects.

GREEN TEA OR CHINESE TEA Camellia sinensis
Where It Comes From: Leaf buds and young leaves of the tea plant; native to Southeast Asia.
Forms and Dosage: Capsule, tablets or tea; typically 125 mg to 250 mg of catechins (a group of beneficial tea compounds) or equivalent to three cups of tea.
What It's Supposed to Do: Fight inflammation.
What We Know: Green tea contains polyphenols, antioxidants that may fight inflammation.
Studies: In several animal studies, green tea appears to exhibit a variety of anti-inflammatory responses. In in-vitro studies, it has reduced inflammation and slowed cartilage breakdown. No human studies show green tea to be effective for arthritis.
What to Watch For: Stomach upset and constipation. Because green tea contains caffeine, large doses should not be taken if you are pregnant, nursing, have depression, anxiety disorder, ulcers, a heart condition, kidney disease or high blood pressure. May cause an allergic reaction.

GUGGUL, GUGULIPID, GUGGAL Commiphora mukul
Where It Comes From: The gum resin of the guggul tree, which grows in India.
Forms and Dosage: Extract or pill; dosages vary.
What It's Supposed to Do: Decrease inflammation.
What We Know: Research suggests guggul has anti-inflammatory properties, but no conclusive evidence it is effective for arthritis.
Studies: No human studies on arthritis.
What to Watch For: Stomach upset, headache, nausea, belching and hiccups.
Extras: May reduce cholesterol.

INDIAN FRANKINCENSE, FRANKINCENSE, BOSWELLIA, BOSWELLIN, SALAI GUGGAL Boswellia serrata
Where It Comes From: Gum resin from the bark of the Oswellia tree found in India, Northern Africa and the Middle East.
Forms and Dosage: Capsule or pill; typically 150 mg three times per day.
What It's Supposed to Do: Reduce inflammation and treat RA, OA and bursitis symptoms.
What We Know: Indian frankincense may have anti-inflammatory and anti-arthritis properties, but effects are inconsistent.
Studies: Few clinical studies done. When combined with supplements like turmeric and zinc or turmeric, ginger and ashwagandha, Indian frankincense appears to be effective for OA and RA.
Extras: Indian frankincense may treat symptoms of ulcerative colitis.

KAVA KAVA, KAVA, KAVA PEPPER, TONGA, KAVA ROOT, Piper methysticum
Where It Comes From: Dried root of the kava plant, native to the Pacific Islands and Hawaii.
Forms and Dosage: Capsules, tablets, powder, tea or drink, extract; typically 100 mg of standardized extract three times a day; dried root extract, 70 mg to 240 mg; 140 mg to 210 mg daily of kava's active ingredient, kava-lactone; one cup of tea three times daily.
What It's Supposed to Do: Ease pain and treat depression and anxiety. Sedative and muscle relaxant.

What We Know: Active ingredient is kava-lactones. Commercial kava is generally prepared with 30 percent to 70 percent kava-lactones. Kava affects the brain and central nervous system, and its side effects make kava unsafe for consumption. Recent reports show kava taken at normal doses and for short periods (one to three months) can cause liver disease and even death.
Studies: Clinical studies show kava is an effective anti-anxiety agent, reducing stress and nervousness without being addictive. There is no evidence kava treats arthritis.
What to Watch For: If you take kava, consult a doctor immediately if you have yellow skin, dark urine, light colored stools, tiredness, muscle weakness and red eyes. Long-term use is associated with poor health and dermatitis or dry, scaly, yellow skin. Do not take kava if you have Parkinson's disease or are genetically susceptible to it. Do not take if you are depressed.
Extras: Because of reports of liver toxicity, kava has been recently banned from over-the-counter sales in Germany, Britain and Switzerland. Canada, Australia and the United States have issued warnings against taking kava.

MELATONIN
Where It Comes From: A hormone produced by the pineal gland, which is located at the base of the brain. Commercial melatonin is synthesized in a laboratory or may come from animal pineal glands.
Forms and Dosage: Capsules, liquid, tablets, lozenges and tea; capsules and tablets, 0.3 mg to 5 mg at bedtime for insomnia.
What It's Supposed to Do: Aid sleep, boost immune system, prevent osteoporosis and slow aging.

What We Know: A potent antioxidant, melatonin regulates sleep/wake cycles. It appears to treat insomnia and sleep disturbances related to conditions like fibromyalgia and depression. Aspirin and other NSAIDs can decrease melatonin levels. Not proven safe or effective.
Studies: No good clinical studies on melatonin and sleep disorders, so standard dosages have not been established. Studies show melatonin may help regulate bone growth and strengthen immune system.
What to Watch For: Melatonin should not be taken for more than two weeks. Higher doses or long-term usage require a doctor's supervision. Melatonin may increase or decrease the effects of heart, depression and immunosuppressant medications or supplements like valerian or kava kava. Do not take with alcohol. Do not take if you have an autoimmune disease such as lupus or if you have depression.

MSM (Methylsulfonylmethane)
Where It Comes From: Organic sulfur compound found naturally in fruits, vegetables, grains, animals and humans.
Forms and Dosage: Tablets or powder, topical and oral; typically 1,000 mg to 3,000 mg daily with meals.
What It's Supposed to Do: Reduce pain and inflammation.
What We Know: MSM, an organic sulfur, has been studied for arthritis. Sulfur is needed to form connective tissue.
Studies: A few animal studies have shown MSM may ease inflammation. One small study on humans appeared to show relief of arthritis symptoms. No good, well-controlled human studies to date and no evidence for safety or effectiveness in treating arthritis.

NEW ZEALAND GREEN-LIPPED MUSSEL Perna canaliculus
Where It Comes From: Mussel that comes from New Zealand.
Forms and Dosage: Freeze-dried, concentrated or ground in capsules; 1,000 mg per day (takes four weeks to three months to see effects), then a maintenance dose of 350 mg to 700 mg per day.
What It's Supposed to Do: Relieve inflammation in OA and RA.

What We Know: Although New Zealand green-lipped mussels contain omega-3 fatty acids and other compounds (lyprinol and glycomarine) believed to decrease inflammation, findings have been mixed.
Studies: Studies from the United Kingdom and New Zealand show glycomarine reduces inflammation, lubricates joints and reduces pain in 70 percent of people with OA who take it. Works similar to NSAIDs. Lyprinol and mussel powders without the active carbohydrate ingredient do not show anti-inflammatory activity. (Note: Labels do not say if mussels contain the active component.)
What to Watch For: Diarrhea, nausea, intestinal gas and liver problems. Don't take if you are allergic to shellfish.

PHELLODENDRON AMURENSE
Where It Comes From: Extract from the bark of the phellodendron tree; found in China.
Forms and Dosage: Capsule and tablet; 500 mg to 2 grams per day.
What It's Supposed to Do: Relieve pain and joint stiffness, improve mobility and act as anti-inflammatory agent.
What We Know: There is no evidence it works for arthritis.
Studies: No published studies on animals or humans.
What to Watch For: Safety is unknown.

SAM-e-S-adenosyl-L-methionine
Where It Comes From: A naturally occurring chemical in the body.
Forms and Dosage: Tablets and injection; 600 mg to 1,200 mg daily for OA; 1,600 mg daily for depression. Should not be taken without the supervision of a doctor.
What It's Supposed to Do: Treat pain, stiffness and joint swelling, improve mobility, rebuild cartilage and ease symptoms of fibromyalgia, bursitis, tendonitis, chronic low back pain and depression.
What We Know: SAM-e is an effective anti-inflammatory and analgesic for people with OA. It is comparable to NSAIDs, but with fewer gastrointestinal side effects. Symptom relief from SAM-e may take up to twice as long as from NSAIDs, but benefits last longer, continuing after supplementation ends. “Loading” doses, initially starting out with a high dose and then dropping to lower doses later, may work as maintenance. SAM-e works closely with the B vitamins B12, B6 and folate, so it is important to get enough of the B vitamins when taking this drug.
Studies: Over the last two decades, multiple clinical trials involving thousands of people have shown SAM-e to improve joint health and treat OA. It has been found to be equal to NSAIDs in clinical studies. Most of this research has been done in Europe, where SAM-e is sold as a drug. Several studies suggest SAM-e repairs and rebuilds cartilage. These studies however, have only been done in vitro and in animal models. No good human clinical evidence for this.
What to Watch For: High doses can cause flatulence, vomiting, diarrhea, headache and nausea. Avoid if you have bipolar disorder (manic depression). May interact with antidepressive medication. Avoid if you are taking monoamine oxidase inhibitors (MAOI). SAM-e may worsen Parkinson's disease.

SHARK CARTILAGE, CARTILAGE
Where It Comes From: Ground cartilage of sharks caught in the Pacific Ocean.
Forms and Dosage: Capsules, tablets, extract and powder; typically 500 mg to 4.5 grams given in two to six doses daily. Do not mix shark cartilage extracts and powders with acidic fruit juice, such as orange, apple, grape or tomato, because it will decrease effectiveness.
What It's Supposed to Do: Ease pain and inflammation of arthritis and psoriasis.
What We Know: Shark cartilage is composed of collagen, water, calcium, phosphate and some chondroitin sulfate.
Studies: Preliminary animal and in vitro research suggests shark cartilage may have anti-inflammatory and pain-relieving effects. No well-designed human studies. Shark cartilage applied topically may subdue psoriasis.
What to Watch For: Nausea, vomiting, stomach upset, constipation, bloating, low blood pressure, dizziness, high blood sugar, high calcium levels and fatigue.

ST. JOHN'S WORT Hypericum perforatum
Where It Comes From: The yellow flower, leaves and stem of the St. John's Wort plant, which is native to Europe and grows wild throughout the United States.
Forms and Dosage: Extract in the form of powder (dried), liquid or tablet, capsules and tea; extract, typically 900 mg daily.
What It's Supposed to Do: Act as an antidepressant drug and reduce inflammation and muscle pain.
What We Know: St. John's Wort's mood-elevating properties were believed to be from active ingredients hypericin and hyperforin, chemicals that raise levels of serotonin, a chemical found in the brain. Serotonin levels may be low in people who are depressed and possibly in those who have fibromyalgia. New research, however, now suggests the whole preparation (and not just the two active ingredients) is more effective. Benefits can take up to four weeks. People taking St. John's Wort may exhibit withdrawal symptoms when they stop. No scientific evidence showing that St. John's Wort is effective for reducing inflammation. Although St. John's Wort taken alone is considered safe, it is potentially dangerous if taken with prescription medication, which has caused the FDA to issue a warning about taking the herb.
Studies: Small studies show St. John's Wort treats mild to moderate depression. One recent large well-controlled study found St. John's Wort not effective for people with severe depression.
What to Watch For: Insomnia, restlessness, anxiety, irritability, stomach upset, fatigue, dry mouth, dizziness or increased sensitivity to sunlight. Do not take if you are taking any kind of prescription medication. Do not take if you have Alzheimer's disease, HIV, depression, schizophrenia, infertility or bipolar disorder. Do not take for longer than two months.

STINGING NETTLE Urtica dioica
Where It Comes From: The leaves and roots of the stinging nettle plant, a tall, herbaceous, stalk-like plant that can be found throughout the world.
Forms and Dosage: Tea, tincture, extract or whole leaf; tea, one cup, three times a day; tincture, 1 ml to 4 ml three times a day; nettle leaf applied directly to the skin.
What It's Supposed to Do: Reduce inflammation, aches and pains of OA.
What We Know: Stinging nettle leaves are covered in tiny hairs that produce a chemical that irritates the skin when touched. They contain a number of phytochemicals (beneficial plant substances) that may relieve pain and fight inflammation.
Studies: There are a few studies that show nettle leaf, taken as an extract, along with conventional NSAIDs, can allow users to reduce their dosages of NSAIDs. Two small studies, one on people with hip OA and the other on subjects with thumb joint pain suggest nettle leaves, applied topically, can lessen pain.
What to Watch For: Since nettle is high in vitamin K, it can increase the risk of clotting. May decrease the effects of blood thinners, diabetes and heart medications, and lower blood pressure. May also increase the effects of tranquilizers and sedatives. Avoid if you have kidney problems.

THUNDER GOD VINE Tripterygium wilfordii
Where It Comes From: Leaf and root of a vine-like plant from Asia.
Forms and Dosage: Extract; 30 mg of thunder god vine extract daily used in studies. No standardized safe doses have been established.
What It's Supposed to Do: Reduce pain and inflammation and treat symptoms of RA, lupus and other autoimmune diseases.
What We Know: Used in Chinese medicine for years, thunder god vine shows evidence of suppressing the immune system.
Studies: Most of the research related to RA was done on laboratory rodents. However, one study on people with RA done in China found thunder god vine to relieve symptoms in people taking NSAIDs. Another Chinese study showed thunder god vine to significantly improve symptoms of people with systemic lupus, leading to a reduction in conventional medicine.
What to Watch For: Stomach upset, skin reactions, temporary infertility in men and amenorrhea (lack of menstruation) in women. Should not be used by people taking immunosuppressive drugs, like prednisone. The leaves and flowers of this plant are highly toxic and can cause death, so preparation should only be made from the root.

TURMERIC—Curcuma longa, Curcuma domestica
Where It Comes From: A yellow-colored powder ground from the roots of the lily-like turmeric plant. It is a common ingredient in curry powder. The turmeric plant grows in India and Indonesia and is related to the ginger family.
Forms and Dosage: Capsules or spice; capsule, typically 400 mg to 1,000 mg three times per day; or 0.5 gram to 1 gram of powdered root up to 3 grams per day.
What It's Supposed to Do: Reduce pain, inflammation and stiffness related to RA and OA; treat bursitis.
What We Know: Traditionally used in Chinese and Indian Ayurvedic medicine to treat arthritis; the active ingredient in turmeric is curcumin.
Studies: One small human study that used a combination supplement of turmeric, boswellia and zinc found a decrease in pain associated with OA. Two other studies using a combination of turmeric, boswellia, ginger and ashwagandha relieved pain and inflammation in RA. Effectiveness alone is unknown.
What to Watch For: High doses can act as a blood thinner and cause stomach upset. Do not take if you have gallstones.
Extras: Known as a cleansing agent, turmeric is often used as a digestive aid in India.

TYPE II UNDENATURED CHICKEN COLLAGEN, CHICKEN COLLAGEN, CHICKEN TYPE II COLLAGEN, TYPE II COLLAGEN
Where It Comes From: A protein derived from chicken sternum cartilage.
Forms and Dosage: Type II chicken collagen is undenatured (in its natural state), as opposed to denatured or hydrolyzed collagen (see collagen hydrolysate). To date, only one manufacturer produces undenatured Type II chicken collagen. Recommended dosage is 10 mg daily, which is typically sold as a 40 mg capsule, providing 10 mg of undenatured type II collagen.
What It's Supposed to Do: Works via the immune system to relieve pain, inflammation, swelling and stiffness of RA, OA, JRA and gout.
What We Know: Small amounts of collagen, taken by mouth, appear to reduce RA symptoms by suppressing the autoimmune response. Research has shown results are very dose sensitive.
Studies: Several studies from Harvard University and Germany have shown positive effects on the symptoms of RA. One of the largest studies involving a multi-centered, double-blind, placebo controlled group of 274 people with RA tested four dosages: 20 mcg, 100 mcg, 500 mcg or 2,500 mcg (2.5 mg). Only the lowest dose demonstrated significant improvement, which included a reduction in inflammation and joint pain. High doses had no effect. Undenatured Type II collagen also appeared to reduce swollen and tender joints in JRA subjects in a three-month study from Harvard. Here, patients were given 100 mcg daily the first month, followed by 500 mcg for the next two. Several German studies on RA and undenatured Type II collagen have also found a positive outcome. To date, there are no clinical studies on OA and undenatured Type II collagen, however, there is some speculation that it would be beneficial.
What to Watch For: No known drug interactions or side effects in small doses; large doses can cause nausea. Do not take if allergic to chicken or eggs.
Extras: Only undenatured Type II chicken collagen has been shown to be safe and effective in clinical trials. Denatured or collagen hydrolysate has not. If you are unsure of the type of collagen, look at dosages. Generally, undenatured collagen is only required in small amounts, for example, one 40 mg capsule daily, providing 10 mg of undenatured type II collagen. Denatured collagen products can recommend up to 2 grams or more, which can sometimes mean four tablets a day.

VALERIAN—Valeriana officianalis
Where It Comes From: The dried root of the perennial herb valerian.
Forms and Dosage: Capsules, tablets, extracts or tea; 300 mg to 500 mg of valerian extract daily (maximum dose is 15 grams of root per day); one cup of tea taken several times a day. Tea is made by steeping 2 grams to 3 grams of dried root in boiling water for five to 10 minutes.
What It's Supposed to Do: Treat insomnia and ease muscle and joint pain.
What We Know: Valerian works as a mild sedative and sleep agent. No known effects on muscle or joint pain and arthritis. Active ingredients in valerian are unknown.
Studies: Clinical studies have shown valerian to alleviate insomnia.
What to Watch For: Headache, excitability, uneasiness and insomnia. Do not drive or operate machinery while taking valerian. Do not take with alcohol, barbiturates, tranquilizers or other sedative-type drugs/herbs. Do not use longer than one month.

WHITE WILLOW, WILLOW BARK, WHITE WILLOW BARK—Salix Alba
Where It Comes From: Bark of the white willow tree.
Forms and Dosage: Tea, liquid extract or tincture. Dosage varies.
What It's Supposed to Do: Reduce pain and inflammation, ease muscle and joint aches related to gout, ankylosing spondylitis, OA and RA.
What We Know: Active ingredient in willow bark is salicin, which is chemically similar to salicylates, the main active ingredient in aspirin and other NSAIDs. Thus, willow bark has the same potential benefits and adverse reactions as aspirin and NSAIDs. Used appropriately, it is considered safe and possibly effective. However, due to other compounds in willow bark, it takes much longer to act than salicylates. Variations in salicin levels among the 300 willow species means no one knows how much salicin is in willow bark. In general, even high-quality willow bark contains only small amounts of salicin. According to one researcher, it would take about 1.5 gallons of willow bark tea per day to obtain the same amount of pain relief of 4.5 grams of aspirin, the average daily dose used to treat arthritis and related conditions.
Studies: One clinical study showed moderate doses of chemically standardized willow bark extract to have an analgesic effect on people with OA. A few studies have also found 120 mg to 240 mg of willow bark to be safe and effective for relieving lower back pain.
What to Watch For: Do not take if you have aspirin allergies. Do not give to children under 18 years of age who could develop Reye's syndrome, a fatal illness that strikes children. May increase the effects of blood-thinning drugs or supplements.

WILD YAM—Discorea villosa
Where It Comes From: The root and bulb of the herbaceous vine of a plant that grows wild in much of the United States.
Forms and Dosage: Tablet or cream; no typical dosage.
What It's Supposed to Do: Promoted as the “natural” source of DHEA and used for RA, lupus and a host of other illnesses.
What We Know: Although wild yam contains steroids, they are not in a form your body can use. These raw ingredients must be converted in a laboratory for your body to access them. Possibly safe, but ineffective. There is no evidence that wild yam helps arthritis-related symptoms.
Studies: No well-controlled studies on animals or humans.

SierraSil—Natural hydrothermally altered volcanic mineral composite
Where It Comes From: Pristine mineral deposit from the High Sierra Mountains.
Forms and Dosage: Powder, capsule, tablet, liquid, food stuffs; standard form Clinical Strength dosage is a minimum of 2 grams per day on an empty stomach. Micronized form (median area under the curve is ˜4 microns) Clinical Strength dosage is a minimum of 1 gram per day on an empty stomach. Lesser quantities can be used as a source of trace minerals.
What We Know: SierraSil has an acidic pH that promotes effective ionization, absorption and bioavailability. SierraSil works through myriad mineral dependent pathways to act as pH maintenance buffers, function as electrolytes, aid in enzymatic processes, promote energy production and healthy metabolism, promote effective antioxidant activities, and promote healthy immune function, promote molecular synthesis (formation of molecules, membranes and tissues), support structural integrity of the body, and reduce or eliminate the need to initiate an inflammatory response.
Studies: Both a human pilot study and a randomized, double blind, placebo controlled clinical study found SierraSil works quickly to promote joint health, mobility, flexibility, increased range of motion and increased joint strength, while protecting cartilage from wear and tear and breakdown.

AlgaeCal—A wild harvested plant-source of calcium and magnesium augmented by a complete array of macro and trace minerals.
Where It Comes From: AlgaeCal is wild harvested on private, pristine and protected areas of the coast of South America.
Forms and Dosage: Recommended intake levels for Full Strength range from 2400 mg to 3200 mg per day. Lesser quantities are used as a vegetarian source of plant minerals.
What We Know: AlgaeCal is a viable source of calcium (˜30%) and Magnesium (˜10%) along with a full complement of other macro and trace minerals. AlgaeCal works through myriad mineral dependent pathways to act as pH maintenance buffers, function as electrolytes, aid in enzymatic processes, promote energy production and healthy metabolism, promote effective antioxidant activities, and promote healthy immune function, promote molecular synthesis (formation of molecules, membranes and tissues), promote healthy bones, tendons, cartilage and muscles, and support the structural integrity of the body.
Studies: Clinical study in progress.
   Obesity

Increased Fat Storage: A “Prehistoric” Perspective on Genetically Mandated Survival Behavior—In consideration of the protective ability of our bodies to increase fat storage, we must review a few simple facts. Without our ability to increase fat storage, we as humans would have never survived. Ironically, whereas today the quantity of food is plentiful (although quality of nutrition is questionable), our goal is to turn away food. In contrast, in the time of our prehistoric ancestors, the hunter-gatherers did not have a plentiful food supply. For example, when pristine sources of nutrient-rich berries and roots were in season and when wild animals were not hibernating, our ancestors ate well and “they fattened up”. However, when these foods were not available, they relied on the stored fat to see them through the lean times. To help us understand the importance of our ability to bolster fat storage, two biological functions assisted our prehistoric ancestors as they struggled to survive this perpetual cycle known as “feast” and “famine”. When there is an abundant supply of pristine quality food, our bodies efficiently store fat, and during times of a lack of food, our metabolism slows down. Scientists believe that abundant food induced efficient fat storage in our ancestors and when there was less fat their metabolism slowed to adjust to the smaller quantities programmed to adapt their metabolic rates to food intake. Those who survived were “blessed” with “fat-storage” genes, while those who lacked these genes perished. This suggests that the survivors passed their “thrifty” genes on to future generations—to you and me. Within the realm of modern times these ancient genes evolved over thousands of years and ultimately forced our bodies to store energy from nutrient-deficient concentrated sugars, processed carbohydrates, and adulterated fats to survive the famine that chronic intake of these types of low quality “foods” simulate. Today we are faced with an obesity epidemic, which contributes to an estimated 300,000 deaths in people who die prematurely from this disease. In fact, obesity is a contributing risk factor for four of the seven leading causes of death. The Center for Disease Control has stated that Obesity is the number one health risk, greater than a lifetime of smoking, drinking and poverty (Public Health, 2003). Obesity in the United States is doubling every five years and the Institute of Medicine has declared war on the nation's “obesity epidemic”. To make sense out of all of this, we must understand that in today's modern world, no longer do we struggle through periods with very little food. Instead, we live in a perpetual calorie rich-nutrient deficient food feast with a fast-food chain virtually around the corner for all Americans. This means that we are perpetually simulating a famine, continually maintaining our bodies in “fat storage mode”; except when we go on “low-fat diets” or any deprivation-based programs (from our already nutrient deficient diets), our brain loses control and there is an overwhelming call to “eat”. It is this threat of famine (survival insurance) that amplifies protective fat storage signaling with a concomitant down-regulation of the basal/resting metabolic rate. The primary objective of commercial weight loss programs is “rapid weight loss.” The numerous array of stimulatory, deprivation-based and metabolic inhibitory tactics employed to achieve these objectives are usually pursued without regard for or knowledge of the impact on health, the body's natural genetically mandated homeostatic response to such tactics, or the fact that depriving the body of resources essential to maintain health is counterproductive. Essentially, these types of tactics simulate circumstances equivalent to a famine and induce genetically programmed energy conservation responses. In addition, at some point in the energy conservation sequela, increased appetite is a natural and automatic consequence. Alarmingly, many of these tactics are approved, administered and/or supervised by medical or health professionals. While initially appearing to promote “weight loss” (phase 1), such tactics are destined to fail as gene-induced recalibration of energy management and storage instructions homeostatically adjusts to the artificially imposed influence of such tactics, generally by lowering the basal metabolic rate, increasing energy storage requirements and promoting increased fat retention (phase 2) [Tataranni et al. 2001]. Chronic and repeated attempts to lose weight with such tactics are referred to as the “yo-yo” rebound weight gain effect. This phenomenon is responsible for ever-increasing frustration, anxiety and a sense of helplessness caused by the out-of-control “weight loss”/gain juggernaut. Thus, in all of us, there is a rebound effect, which reacts by quickly regaining the lost weight in preparation for the next food shortage, just as it did for our prehistoric ancestors.

A 1996 article in Obesity Research sums up the problem: “[The] modern western lifestyle appears to provide the social and environmental conditions that favor maximum expression of underlying individual genetic differences and the susceptibility to promoting the sequela of events that lead to-obesity.” This is an important view because we now know that in today's society, with its highly processed foods, chemicals and pollution, with regard to metabolic effects, the body's instinct is to prepare for and defend against famine, but there is even a more important facet to the genetic propensity to store excess fat and it does not reside in genes which control fat storage, and/or resting metabolic rates. These genes are termed “reward genes”.

Compulsive bingeing—the role of dopamine & other genes—Obesity is a disease that comes in many forms. Once thought to be primarily environmental, it now is considered to have both genetic and environmental components. In a Swedish adoption study, for example, the weight of the adult adoptees was strongly related to the BMI of the biological parents and to the BMI of the adoptive parents. Other studies of adoptees and twins suggest heredity is an important contributor to the development of obesity, whereas childhood environment has little or no influence. Moreover, the distribution of fat around the body also has been found to have heritable elements. The inheritance of subcutaneous fat distribution is genetically separable from body fat stored in other compartments (among the viscera in the abdomen, for example). It has been suggested there is evidence for both single and multiple gene anomalies. In fact according to our laboratory, in conjunction with David Comings of the City of Hope National Medical Center, at least twelve different genes have been associated with obesity providing a 33 per cent contribution to the overall variance.

Given the complex array of metabolic systems that contribute to overeating and obesity, it is not surprising that a number of neurochemical defects have been implicated. Indeed at least three such genes have been found: one associated with cholesterol production, one with fat transport and one related to insulin production. Other genes include human chromosome 2, uncoupling protein 2 and the APO-D genes. The ob gene and its product the leptin protein have also been implicated in regulating long-term eating behavior. Another protein, glucagon-like peptide 1(GLP-1) has been found to be involved in the regulation of short-term eating behavior. The regulation between leptin and GLP-1 is not known. The ob gene may be involved in the animal's selection of fat. But perhaps not in the ingestion of carbohydrates, which appears to be regulated by the dopaminergic system. It may be that the ob gene is functionally linked to the opioid peptodergic system involved in reward. Whatever the relation between these systems the complexity of compulsive eating disorders suggests that more than one defective gene is involved. Indeed, the relation between compulsive overeating and drug and alcohol addiction is well documented. Neurochemical studies show that pleasure-seeking behavior is a common denominator of addiction to alcohol, drugs, and carbohydrates.

Variants of the dopamine D2 receptor gene appear to be risk factors in obesity. The A1 allele was present in 45 percent of overweight subjects as compared to 19 percent of non-overweight subjects. Furthermore, the A1 allele was not associated with a number of other metabolic and cardiovascular risks, including elevated levels of cholesterol, and high blood pressure. In contrast, when the subject's profile included factors such as parental obesity, a later onset of obesity and carbohydrate preference, the prevalence of the A1 allele rose to 85 percent.

Genotyping For Customized Nutraceuticals—Overeating is a biogenetic condition that comes in many forms. Once thought to be primarily environmental, it is now considered to have both genetic and environmental components. Given the complex array of metabolic systems that contribute to overeating, it is not surprising that a number of neurochemical defects have been implicated. It is important to realize that carbohydrates cause the release of the pleasure-inducing brain chemical dopamine. Because of the complexity of compulsive eating disorders, it is likely that more than one defective gene is involved. Indeed at least twelve genes (involved in the neurochemistry of brain reward) have been associated with morbidly overweight people. Moreover, three metabolic type genes have been identified: one associated with cholesterol production; one with fat transport; and one related to insulin production. In studying genetically obese mice, a Leptin gene was isolated that plays a role in regulating the brain center that controls eating behavior. There is general agreement that other pleasure-inducing substances such as alcohol and nicotine, like glucose, work through the dopaminergic pathways of the brain. This shows the common genetic thread of multiple addictions. There are a number of investigators that have observed a significant association between an abnormal form of gene known as the dopamine D2 receptor gene (involved in expressing the actual number of dopamine D2 receptors) and obesity; time of onset of obesity; carbohydrate preference or craving ; high body mass index; percent of body fat; co-morbid drug abuse; energy expenditure, hyperphagia (overeating) and low dopamine D2 receptors. Since obesity and overeating including bingeing behavior is polygenic, many genes have been identified. These will be briefly discussed, but emphasis will be directed to certain candidate genes having impact on the brain reward cascade. The reason for this is that the subject of genes and obesity is an opus book in by itself and genetics, while very important, is not the sole intent of this book.

In his book, the “The Gene Bomb”, medical geneticist David E. Comings suggests that while genes and environment play a significant role in complex behavioral disorders including but not limited to a number of impulsive, compulsive, and addictive behaviors, the rate of selection of certain genes will progressively increase, based on the age of first pregnancy. Accordingly, Comings believes that as a result, the rate of selection of these genes will potentially destroy the species from within. While this book addresses problems dealing with ADHD and substance seeking behaviors, the concept may have impact on the obese community because obesity is life threatening and could also destroy society. To help us understand our genetic legacy and societal impact Comings had this to say, “In the near future, all the genes involved in increasing one's risk of developing alcoholism, drug abuse, sugar craving, ADHD, aggressive and impulsive conduct and related disorders, will have been identified, and the relative risk of developing these disorders for individuals with varying combinations of the mutant genes will be known.” The question to ponder then; will gene testing be important to individuals if we indeed discovered the genes involved in contributing to obesity and related behaviors? The second question will be whether these genes map to not only obesity but to sugar craving and are these genes associated with what I have coined “Reward Deficiency Syndrome”.

Moreover, in our attempt to understand the meaning of obesity we must be cognizant of the fact that although many still regard obesity as a problem of self-control, obesity is a multi-factorial process that is believed to be catalyzed, at least in part, by certain nutrient deficiencies (i.e. magnesium, chromium, potassium, zinc, etc.) that contribute to the development of Syndrome X sequela, which intensify insulin resistance, consequential genetic polymorphisms, and metabolomic dysequilibrium resulting in symptoms characteristic of this metabolomic, medical, and genetic disorder. For example, magnesium is essential for calcium metabolism. Calcium is required for DNA synthesis, an important downstream event from magnesium's involvement. Further, magnesium is required for insulin and insulin-like growth factor I (IGF-1) function. Insulin and IGF-1 have related roles in the regulation of cell growth and metabolism, however, insulin is primarily involved in such physiological processes as glucose transport and synthesis of glycogen and fat, whereas, IGF-1 has been shown to be more potent in stimulating cell growth by increasing DNA synthesis and in promoting cell differentiation. IGF-1 Receptor has been shown to be important in the onset and maintenance of the transformed phenotype in vivo and in vitro (Kaleko et al., 1990; Baserga, 1995). Furthermore, all enzymatic reactions that involve ATP (adenosine triphosphate) have an absolute requirement for magnesium. Magnesium metabolism is interactively linked to calcium, potassium, and sodium metabolism and controlled/regulated by the kidneys and gastrointestinal tract. As such, magnesium has an important role in IGF-1 function and is essential for healthy insulin, glucose and energy metabolism; pH homeostasis; and a multitude of other biological systems involved in maintaining healthy body composition. Therefore, deficiencies in essential nutrients (i.e. magnesium) place undue stress on metabolomic homeostasis and amplify the consequences of gene polymorphisms. A more serious problem is that mineral losses (i.e. magnesium and potassium) may be overlooked as serum concentrations must remain within normal ranges while muscle concentrations (critical for cellular energetics) can be considerably reduced. Moreover, in long-term diuretic induced Mg and K deficiencies, oral magnesium intake reestablished normal Mg as well as K status. Furthermore, the normalization of muscle Mg and K was accompanied by a restoration of the concentration of Na/KATP-pumps. Therefore, the events that lead to obesity involves a complex interplay of survival need-induced gene expressions and their interactive influences on our endocrine and immune systems, nutrient deficiency-amplified expressions of gene polymorphisms, general cravings, how we burn energy, store fat and regulate our appetites. Further, the hormone Ghrelin plays an important role in stimulating food intake by activating hypothalamic neuropeptide Y (NPY) nerurons/agouti related peptide neurons. Ghrelin also stimulates growth hormone (GH) secretion through its action as an endogenous ligand for the hypothalamic-pituitary GH secretagouge receptor. In addition, ghrelin has been shown stimulate neurogenesis and is implicated in longterm energy homeostasis. Whether genetic or not, the diet industry collects more than $50 billion a year promoting the idea that we can achieve an ideal body shape if we only follow a few simple rules. This runs contrary to the emerging scientific/medical view that our body shape and weight are very tightly regulated, that genes expression controls much of this regulation, and that it is very difficult to change.

During each decade of life, the average adult American eats 10 million calories—an enormous amount of energy—and gains only a few pounds. From this, we can calculate that the calories eaten are 99.83% of the calories burned. Someone who is “only” 99.5% efficient can store fat at triple the usual rate. These numbers show that appetite is precisely regulated in humans. This suggests that obesity is a problem where hunger and metabolism are not properly coordinated and that in order to understand how appetite is controlled, we must begin to identify genes and treatment targets thereof.

Frequently Asked Questions about our Genes & Obesity

Can obesity run in families?
There are numerous studies that have provided evidence that obesity runs in families. In fact, the genetics of human obesity is receiving increasing attention as exemplified by the number of papers published on the subject since 1980. The growth has been particularly remarkable in the 1990's. There is ample evidence from twin and family studies that genes play a role in obesity. Most of these genes are susceptibility genes. These genes are necessary, but not sufficient alone, to cause obesity.

When obesity runs in families, is the reason genes or environment?
As with most common disorders, both genetics and environmental elements play a role in obesity. In fact, obesity is a very good example of a strong interaction between genes and environment. Obesity only occurs when a person eats more calories (especially nutrient-deficient calories) than they burn and when their ability to process and burn calories is inadequate and/or impaired. Moreover, obesity does not occurs when nutrient-rich food is consistenly scarce; a condition typical for most of human history and in many parts of the world today. Under scarce food conditions, our environment exerts a controlling influence on our gene expression. Fortunately, the Western World today has an abundance of food for most persons. Interestingly, it is believed that when there is an abundance of food it “brings out” the gene variants that contribute to obesity.

The following table taken from the WebMD site may help show this interaction:

TABLE 2

| Factors | Genetic Factors | Environmental |
|---|---|---|
| Calories eaten | Leptin (decrease) | Food poisoning (decrease) |
| (appetite) | Eating Disorders | Thanksgiving (increase) |
| | (decrease) | Amphetamines (decrease) |
| calories burned | Thyroid (increase) | Exercise (increase) |
| (metabolism) | | Temperature (Increase and/or |
| | | decrease) |

TABLE 2 from WebMD shows that genes and environment each affect both factors of obesity. For example, thyroid hormone is made by the body's thyroid gland. Genes control how much hormone the gland producers, and the hormone influences the burning of energy. On the other hand, someone who takes amphetamines (“speed”) will lose their appetite and hence lose weight, especially lean mass, which is their critically needed energy processing equipment. Consequently, this process creates a crisis that can result in excessive aberrant and unhealthy behaviour. Eating disorders, such as anorexia nervosa, have both genetic and environmental components. Their complex effects on appetite ultimately result in fewer calories becoming available to the body, exacerbating the gene-expressed survival crisis response.

Are there many obesity genetic disorders?
There are many disorders, caused by defects in individual genes that have obesity as one feature of the disorder. These disorders are rare (1 person in 25,000), but they illustrate clearly that genes can play a very significant role in body weight. I am quite sure that most of you probably do not care to know all the genetic names involved, however, for those of you curious and may be victim of one or more of these disorders the list may be of interest. These genetic disorders include (not complete): Achondoplasia, Adiposis dolorosa, Posterior polymiphous corneal dystrophy, Momo Syndrome, Prader-Willi syndrome, Schinzel syndrome, Polycystic ivarian syndrome, Alstrom Syndrome, Badet-Biedi, Biermond syndrome, Cohen syndrome, Cushing Syndrome, Pickwickian syndrome, Short stature-obesity syndrome, Summit syndrome, Borjeson-Forssman-Lehmann syndrome, Chroideremia with deafness & obesity, Wilson-Turner syndrome. There are approximately more than 30 genetic disorders, but most of these disorders are rare, and their genes have not been found.

How many obesity predisposition genes have been identified?
As of October 2000, there have been forty-seven human cases of obesity caused by six different single gene mutations; twenty-four Mendelian disorders exhibiting obesity as one clinical manifestation; from animal research we know of 115 genetic sites associated with obesity and related problems; 130 studies now report positive associations of 48 specific genes;59 different chromosomal sites have been linked to obesity; there are now over 250 genes, markers, and chromosomal regions that have has been associated with obesity and related behaviors.

Is obesity mostly genetic or is it mostly environmental?
Studies of twins provide the clearest evidence for genes and environment both having a role. In 1997, researches examined data from 25,000 pairs of twins and a total of 50,000 family members. They found that, on an average, obesity is 67% genetic and 33% environment.

What genes are involved in obesity?
There are many excellent reviews on the genetics of obesity. The risk of becoming obese has a strong genetic component. There is no single gene that causes obesity and/or any related behavior including sugar craving. It is likely that many genes are involved in risk for obesity and related behaviors. This is a very complex area of research with many conflicting results. Our goal here is to provide an incomplete list of certain genes, which have been considered with some degree of confirmation by a number of investigators. Most scientists would agree that there are at least 40 genes currently under study, one on every chromosome (46 pairs). Obesity researchers believe that there may be two basic categories involved in obesity gene risk: rare genes and common genes. A few genes have variants that are powerful enough to cause obesity all by themselves. For example, rare variants of the POMC gene on chromosome 2 and another gene called the PC1 gene on chromosome 5 can cause obesity, among other problems. The leptin gene in published families has been found to increase appetite and obesity. Growth Hormone Secratagogue Receptor (GHSR) is located on chromosome 3q26.31 and is a target of ghrelin, which becomes the endogenous ligand for GHSR. Ghrelin is an orexigenic gastric hormone that induces NPY release and inhibits proinflammatory cytokines via its antagonistic relationship with leptin. NPY is a potent appetite stimulator controlled by ghrelin and leptin and also acts as a mediator of immune function. Ghrelin is increased during caloric restriction and fat depletion to increase appetite and food intake. Human ghrelin plasma levels affect muscle status, energy dynamics and are inversely correlated with body mass index (BMI); extremely high levels are observed in patients with anorexia nervosa, and obese individuals have reduced levels. A role of ghrelin in the regulation of lipid and energy metabolism is suggested by fat gain independent of changes in food intake during exogenous ghrelin administration in rodents. Recall that a proposition of this patent is that impaired immune system function contributes to gene-regulated metabolomic survival behaviors including (when possible) increased fat storage, lowering of the metabolic rate and increased appetite, factors which also can contribute to obesity under the appropriate circumstances. This relationship is why appetite regulatory factors are adversely amplified or depressed during various states of compromised and/or strained immune function. Among genotyped Ghrelin single nucleotide polymorphisms (GHRL SNP), the variant allele for GHRL −4427G>A was inversely associated with all cases of Non-Hodgkins lymphoma. Nutrient deficiencies that lead to obesity-based neurogenobolic behaviors also affect the type, competence and magnitude of immune system responsiveness. Moreover, variants of another gene, the MC4R gene on chromosome 18, are somewhat more common. Inheriting one copy of certain gene variants (i.e. MC4Rgene) causes obesity in some families. These variants are found in 3-5% of very obese persons (BMI over 40). Notably, not all individuals with uncommon variants of MC4R are obese. Researchers are investigating all the chromosomes and have now found that gene variants on chromosome 20.11,10.5 and 2 seem to be most promising. In a recent study on 388 morbid obese patients with a mean BMI of 52 who underwent gastric banding surgery a number of gene variants have been associated with either obesity or the rate of the development of obesity. These include two specific genes that have already been linked to diabetes: Beta-AR 3 W64R and ucp-1 a-386g variants. There are other genes that have been identified with food regulation including genes controlling leptin, genes producing leptin receptors, genes involved in the synthesis of opioid peptides and other peptides and a gene called the PCI gene (proconvertase) which is involved in producing alpha-melanocyte-stimulating hormone, which reduces food intake when it binds to its MC4R brain receptor. A major gene that has been associated with obesity is the dopamine D2 receptor gene located on chromosome 11. This particular gene has associatioins with many eating behaviors. Information on this gene will help us understand the program we designed to help combat sugar cravings.

How will discoveries about DNA help people and families with obesity?
As we learn more about our genes and how these genes influence our uncontrollable desire to eat sweet tasting foods, as only one example, from this understanding, new treatments and prevention strategies will emerge. The leptin story is proof that DNA analysis could potentially lead to hope for the millions caught up in the web of obesity. Let me explain by way of example. The leptin gene was discovered in 1994. As researchers worked out the function of the gene, they discovered a previously unknown chemical system used by the body to control appetite and calorie burning. Then scientists actually found some obese humans who had defects in this system. Would you believe that efforts are underway to treat obesity in these individuals using leptin as a major anti-obesity agent. So you see how a simple discovery on a bit of DNA may indeed lead to potential treatment.

When obesity runs in families, why don't all family members have it?
This is an age-old question. To understand the answer a simpler question would be—Why don't all members of my family look like me? As we know all members in a family do not have the same height, weight, and face. Using this as an analogy, it makes sense that they don't all have the same conditions and diseases including carbohydrate bingeing behavior. As stated earlier, usually genes do not cause the disease and for most complex behavioral disorders it is a combination of both genetics and environment. Any phenotype [P](outward characteristic including obesity) is always equal to one's genes[G] (genome) and the environment [E](many elements including stress). Thus a simple formula could explain the above question: P=G+E. In essence, some family members will inherit genes that predispose to carbohydrate bingeing, and others will not. Some family members will be exposed to environmental agents that trigger disease, and others will not.

What environmental factors are involved in obesity?
We will not address all of the environmental factors that may have a role in obesity and related disorders. There are a few factors that stand out, including:
   Physical activity—Would you believe that physical activity accounts for 20-50% of all energy we burn. The rest is burned as our organs work, however, there is no exact estimate it varies between different people and different ethnic groups. I am sure that you may have noticed that people of the same obesity level tend to have the same amount of inactivity, and that thinner people are more active. For example, it was found that children who watch television for 5 hours or more each day were 5.3 times more likely to be obese if compared to children who watch for less than 2 hours a day.
   Energy Intake—Here is an interesting fact concerning will power, although genes play a role in determining how many calories we eat, voluntary control still plays a very important and large role. If we take an extreme example: Sumo wrestlers, to stay obese must consume voluntarily a very high caloric diet.
   Fatty Foods—Fast food can be a person's poison. The effects of modern living changes in physical activity and the availability of high-fat foods are linked to obesity. High-fat foods are important according to some experts, because the brain is relatively poor at sending the “stomach full”, stop eating message when fat foods are consumed. The message here is that while there are a number of reasons for why people overeat, when it relates to high fat food the brain—stop eating connection may be the real culprit.
   Fetal Programming—While research in this area is just beginning, it is known that the nutritional state of a pregnant mother has important effects on the adult health of her child. Here is a good one to ponder. Babies born after the 1944-1945 famine in Holland showed that adult obesity was more likely if famine conditions had occurred during the first two trimesters of pregnancy. This fact, as in ancient times where there was great famines, suggests that the developing infant adjusts its metabolism to favor the storage of energy—a trait that leads to obesity when food is plentiful.

Can the environment change genes?
When we take all these environmental examples into account, it is becoming more clear that the expression of genes can be altered by then environment and that brain manipulation beyond the DNA stage (the RNA stage) can bring about a brain reprogramming. For example, if you constantly stimulate the dopamine D2 receptor with a dopamine agent, even if the person is carrying a DNA form which compromises the normal number of D2 receptors this bombardment at the D2 site signals to the brain and activates the brain's innate machinery through RNA to proliferate more D2 receptors. This kind of manipulation may be the basis of the “Sugar Cure” concept. The point is that by manipulating the environment we cannot change a person's DNA or genes but a gene's impact on a physiological condition can be altered.

Genetic Anatomy Of The Chemical Messenger Link to Obesity and Bingeing—The goal here is not to be burdensome with an enormous litany of scientific facts that support the notion that certain candidate genes, which regulate certain chemical messengers, play important roles in both obesity and carbohydrate bingeing behavior. Instead, it is to provide facts that challenge the belief that will-power alone controls eating behavior. This will be accomplished by simply bulleting some important scientific facts concerning how genes can control neurotransmitters (chemical messengers) and in turn how these messengers influence various aspects of weight management. Since our program involves natural manipulation of the brain reward circuit with amino-acid precursors and certain herbals; and the final common pathway involves dopamine release, subsequent information will only cover the interaction of this chemical messenger, obesity, related physiological processes and bingeing behavior. Multiple genes are involved as well as multiple brain chemicals since-obesity is a complex disorder caused by polygenes and environment.

Neurotransmitters and Obesity: Animal Studies—The literature on eating is very complex. The same chemical element or neurotransmitter commonly will have different effects when administered in low doses versus high doses, centrally versus peripherally, in short-term versus non-predisposed, in overweight versus normal weight versus anorectic animals, as function of paradigm, and so on.

Eating-Stimulatory Neurotransmitters—The eating-stimulatory neurotransmitters include the catecholamine norepinephrine, acting through noradrenergic receptors, GABA, and three classes of neuro-peptides the opioids (endorphins, enkephalins, and dynorphins); the pancreatic polypeptides (neuro-peptide Y and YY), and galanin. These substances, when administered directly into the rat hypothalamus, potentiate eating in satiated animals. Furthermore, chronic administration of certain monoamines (norepinephrine [NE]) and neuropeptides significantly alter daily food intake and weight gain.

Eating-Inhibitory Neurotransmitters—The eating-inhibitory neurotransmitters in the brain include the monoamines, dopamine, serotonin, and gut-brain peptides cholecytokinin-8 (CCK-8), neurotensin, calcitonin, glycogen, and corticotropin-releasing factor. The effects of these neurotransmitters on eating are characterized primarily by a specific change in macro-nutrient selection, rather than an increase or decrease in total food intake. Many peptides, including CCK-8, bomesin, calcitonin, corticotropin-releasing factor, neurotensin, somatostatin, glucagon, and methionine-enkephalin have selective inhibitory actions on macro-nutrients. Leibowitz and associates reported that medial para-ventricular nucleus (PVN) injections of NE in the rat induce a selective increase in carbohydrate ingestion with little or no change in fat and suppression of protein intake. Carbohydrate-craving behavior is observed consistently with chronic stimulation of NE and neuropeptide Y. With regard to the all important monoamine Dopamine (released at the reward center), mixed effects have been observed with regard to the selective actions on macro-nutrient intake.

In contrast, serotonin, in the medial hypothalamus, may selectively suppress carbohydrate intake, while sparing protein intake. Direct serotinergic agonists (e.g., quipazine), indirect serotinergic agonists (e.g., d-fenfluramine), or selective inhibitors of serotonin uptake into serotinergic neurons (e.g., fluoxine) decrease food ingestion in animal studies. Borsini et al. reported that d-fenfluramine (Redux®) reduced the consumption of a sucrose solution in non-deprived rats. Leander demonstrated that fluoxitine suppresses the ingestion of saccharin solutions in normal rats. A similar finding was true for alcohol intake in preferring rat lines (animals genetically bred to prefer alcohol over water). All the above indicates that direct and indirect serotinergic agonists depress a feeding response activated by sweet taste.

Opioid Peptides and Macro-nutrient Selection—Current evidence suggests that the pharmacology of the opioidergic system on eating behaviors is very complex and it would therefore be difficult to a ascribe a generalized role, particularly in view of different effects observed with specific opioid peptides on macro-nutrient selection. In support of the above observation, both increases in food intake as well as decreases in food intake have been observed under a variety of experimental conditions. In short-term experiments, administration of agonists, centrally or peripherally, results in feeding increases.

The results have been far more complicated than expected. In general, chronic administration of antagonists has been disappointing. Naltrexone caused some reduction in binge-eating in bulimics, however, it produced weight gain in anorexic patients. Shimomura et al. observed increased food intake with chronic naloxone treatment and decreased food intake with chronic morphine. Dhatt et al. had similar observations with chronic administration. These observations suggest that while in acute situations opioid agonists increase and antagonists decrease food intake, in chronic situations opposite effects prevail. In this regard, it is noteworthy that the opioid peptides, as well as opiates acting through mu, delta, and kappa receptors, augment ingestion of fat and protein, while actually suppressing the relative proportion of carbohydrates ingested. Tepperman and Hirst showed that upon inducing neonatal reduction of endorphins, rats become overweight. Compared with control animals, these overweight rats chose a greater percentage of their daily calories as carbohydrates and lower percentages as fat and protein.

Inhibitors of Enkephalinase(s) and Craving Behavior—As stated earlier, although it is known that opiates and/or opioids reportedly increase food intake in animals and humans, some papers suggest the opposite-suppression of food intake, especially when one considers macro selection of food sources (i.e., sugar/carbohydrates). To reiterate, Broekkamp et al. reported that infusion of enkephalin into the ventral tegmental A10 area of the brain induces a short-term latency behavioral stimulant effect reminiscent of effects produced by stimulation of the meso-limbic dopamine pathway; this effect is blocked by pretreatment of the opiate receptor antagonist naloxone. This takes on importance in terms of feeding behavior, as feeding has been shown to increase dopamine levels in various brain structures such as the posterior hypothalamus, the nucleus accumbens, and the amygdala.

To reiterate, it is well known that dopamine in sufficient concentration can inhibit food intake. Gilman and Lichtigfeld proposed as an appropriate therapeutic for carbohydrate bingeing (i.e., bulimia) a selective D2 agonist such as bromocriptine [or natural released dopamine], providing D2 occupancy. In this regard, using a push-pull cannula technique, Chesselet et al. were able to induce dopamine release in the “brain reward center” after local application of enkephalin, which suggests regulation by delta receptor stimulation. Indeed Kelotorphan (an inhibitor of the opioid peptide degrading enzyme) may protect against possible CCK-8 degradation by brain peptidases. This important satiety neuropeptide is co-localized with dopamine in the nucleus accumbens, and there is a close interaction between CCK-8, dopamine, and endogenous opioid peptides (like enkephalins).

D2 Receptors and Animal Models—Hamdi et al. studied the specific binding of [3H] YM-09151-2 to investigate the possible differences in age-associated changes in striatal D2 dopamine receptor properties in genetically obese (fa/fa) Zucker rats and their lean littermates. The maximal binding sites of D2DA receptors was found to decline with age in both obese and lean rats: the rate of decline in receptor Bmax was slightly higher in lean than obese rats. However, the Bmax of D2DA receptor in 6-, 12- and 18-month old obese rats was significantly lower compared to the age matched lean rats. The very important interpretation by the authors further support the role of dopamine in obesity. According to the authors, their data indicate that obesity decreases the number of striatal D2DA receptors without affecting the rate at which receptor number decreases with age.

Long-term administration of the antipsychotic drugs known to block D2 receptors such as sulpiride, haloperodol, etc increased body weight in rats. This effect was found to be sex dependent, that is, while female rats were prone to gain weight, male rats did not. In a study conducted by Baptista et al., a linear relationship between dose of sulpiride and body weight gain was found. Also sulpiride increased caloric intake, and both actions were counteracted by the specific D2 agonist bromocriptine. These results confirm that antipsychotic drugs affect feeding and body weight and suggest that hyperphagia and body weight gain might be mediated by blockade of dopamine D2 type receptors.

Hypothalamic neuropeptide Y (VPY) and corticotropin—releasing hormone (CRH) influence feeding and levels of plasma glucose, insulin, free fatty acids, and triglycerides. Treatment of genetically obese, ob/ob mice, with D1/D2 agonists normalizes hyperphagia, body weight gain, hyperglycemia, and hyperlipidemia. Bina and associates (2000) examined whether levels of NPY and CRH immunoreactivity in discrete hypothalamic nuclei are altered in ob/ob mice, and whether dopaminergic treatment reverses this alteration. Such dopaminergic treatment, while normalizing body weight gain and hyperglycemia, also significantly reduced elevated brain levels of NPY and CRH. These findings suggest that dopaminergic D1/D2 coactivation may improve hyperphagia, hyperglycemia, and obesity in the ob/ob mouse, in part by normalizing elevated levels of both NPY and CRH in obese mice. Additionally, the work of Kuo revealed that injection of NPY anti-sense unto brain could modify the anorectic action of repeated S1/S2 agonists, indicating the involvement of NPY. Taken together the present knowledge suggests that both subtypes of D1 and D2 receptors and cerebral NPY are involved in the anorectic action of the dopamine releasing agent amphetamine.

Scislowski and associates reported that a two week treatment with SKF 38393 (a dopamine D1 receptor agonists) plus bromocriptine (a D2 agonist) [BC] acted synergistically to normalize overeating, body fat, hyperglycemia and hyperlipidemia in ob/ob mice. In a more recent study they found that the BC/SKF treatment also increased serum dehydroepiandrosterone (DHEA) sulfate concentrations, an inhibitor of body fat store accumulation. The authors conclude that their findings demonstrate that dopaminergic treatment not only normalizes overeating (hyperphagia) of ob/ob mice, but also redirects several metabolic and endocrine activities, independent of its effects on feeding to improve the obese-diabetic syndrome in ob/ob mice.

More recently, Freeman et. al. studied the effect of glucose on anti-psychotic drug-induced changes in dopamine neuronal activity and suggested that caloric intake may influence antipsychotic drug-induced changes in the population activity of midbrain dopaminergic neurons. In fact, glucose significantly reduced the number of spontaneously active A9 and A10 dopaminergic cells per track in control rats, but significantly attenuated the chronic haloperidol- and clozapine-induced reductions in dopaminergic cells per track.

Dopaminergic Genes and Obesity—In a study by F. Yasuna from Japan, personality is a behavioral pattern, which differs among individuals. E. Kretchmer categorized personality variants according to the concept of fundamental body types. Several lines of evidence suggest that the central dopamine system may underlie the regulation of weight and personality trait. In this study, the authors examined the dopamine D2 receptor (D2R) binding together with body mass index (BMI) and personality trait on the temperament and character inventory in 16 subjects. The data demonstrates a significant relation among the D2R binding in the amygdala, BMI and personality trait of harm avoidance (this is in agreement with other work by Blum et al. showing significant association with D2RA1 variants and harm avoidance). The authors conclude that variation of dopaminergic activity in the amygdala underlies the personality variants related to body type.

In a study by Jenkinson and associates, the association of the dopamine D2 receptor polymorphisms Ser311cys and Taq1A with obesity or type diabetes mellitiis in Pima Indians was evaluated. They found that heterozygotes at the Ser311 CysDRD2 polymorphism had a higher BMI than homozygotes.

Moreover, the atypical antipsychotics have been shown to have superior efficacy compared with typical antipsychotics such as haloperidol, particularly in the treatment of negative symptoms of schizophrenia. However, following clinical use, marked bodyweight gain has been frequently observed with some of the atypical antipychotic drugs. A careful review of the literature from 1966-2000, revealed that relative receptor affinities of the atypical antipsychotics for 5-HT2 and dopamine D2 receptors appear to be most robust correlate of body weight gain. This makes sense because if one blocks dopaminergic sites at the receptor it will increase carbohydrate bingeing. Wetterling suggests that if obesity is a problem in a patient other modalities must be considered for the long term treatment.

In a study, G. N. Thomas evaluated the potential relationship between blood pressure and obesity and dopamine D2 receptor Taq1 polymorphism. Pharmacological data suggest that obesity and blood pressure (BP) may be modulated through the dopamine D2 receptor (DRD2), which may represent and underlying mechanism that links these conditions. Thomas et al. found that the A1 was decreased in hypertensives, compared with controls. In the combined population, systolic, diastolic, and mean arterial BP's were lower in subjects with the A1A1 genotype relative to the A2A2 genotype. However, the DRD2A1 allele frequency increased with increased markers of “gynoidal” or peripheral subcutaneous obesity.

Brain Dopamine Receptors and Obesity Risk—Moreover, dopamine plays a major in the regulation of appetite and growth hormone. Dopaminergic agonists are known to suppress appetite and dopamine D2 receptor antagonists enhance it. Comings found that DRD2 polymorphisms significantly associated with high BMI as well as height.

In another study, Wang and associates found that striatal dopamine D2 receptor availability was significantly lower in ten obese individuals than in lean controls. The availability of the D2 receptors was decreased in obese individuals in proportion to their BMI. Dopamine modulates motivation and reward circuits and hence dopamine deficiency in obese subjects may perpetuate pathological eating as a means to compensate for decreased activation of these circuits. The authors conclude that strategies aimed at improving dopamine function may be beneficial in the treatment of obese individuals.

There also is an increased prevalence of the A1 allele in overweight subjects who have severe alcohol and drug dependence. When obesity, alcoholism, and drug addiction were found in a patient, the incidence of the A1 allele rose to 82 percent. In contrast, the allele had an incidence of zero percent in non-overweight patients who also were not substance abusers and did not have a family history of substance abuse. In an unpublished study Blum and associates also found the A1 allele of the dopamine D2 receptor gene significantly contributes to percent body fat in morbidly overweight subjects. The percent contribution was found to be as much as 45.9 percent of the overall variance, when compared with “super” controls (highly assessed controls-no “reward deficiency” behaviors). Additionally, Comings et al found that the Dopamine D2 receptor A1 allele also associated with overweight young females. Both the ob and the Dopamine D2 receptor gene are additive in contributing to the overall variance of obesity (22 per cent in young females). Thus, the presence of the dopamine D2 receptor gene variants increase the risk of obesity and related behaviors along with other polymorphic genes, some of which have not as yet been identified. In order to investigate the prevalence of the Taq1A1 allele of the dopamine receptor gene in obesity with and without comorbid SUD, a total of 40 patients, from an outpatient clinic were studied. In this sample with a mean BMI of 32, the A1 allele of the DRD2 gene was present in 52% of these obese subjects. Furthermore, it was found that in the 23 obese subjects possessing comorbid SUD, the prevalence of the DRD2 A1 allele was present in 73.9% of the obese subjects compared to only 23.5% in obese subjects without comorbid SUD.

Most recently scientists from Israel and the National Institutes of Mental Health confirmed a genetic variation of the dopamine D4 receptor gene to associate with novelty (or sensation) seekers. Both of these studies set out to test the hypothesis advanced by Cloninger of Washington University that novelty seeking behavior is affected by the way brain cells process dopamine. Epstein and his colleagues at the Herzog Memorial Hospital in Jerusalem found this association in 124 unrelated Israeli subjects. Specifically he found that subjects who scored highest on novelty seeking tended to be compulsive, exploratory, fickle, excitable, quick tempered, and extravagant. They were much more likely to have the longer version of the receptor gene than other subjects. Subjects with the shorter version of the gene scored lower and tended to be reflective, rigid, loyal, stoic, slow tempered, and frugal. In the second study conducted by Benjamin of the laboratory of clinical science, National Institute of Mental Health found similar results in his sample of 315 American subjects, most of them male siblings and other family members. The D2 receptor gene and the D4 receptor gene are fairly close in gene homology and may have similar physiological functions.

In an unpublished work scientists at UCLA found an association between the DRD2 A1 allele and agitation marked by impulsivity, excitability, “hot temper”. These subjects were classified as “sensation seekers.” The recent work of Benjamin and Epstein provide additional confirmation of the relationship between the Reward Deficiency Syndrome behaviors characterized by Blum and associates and the dopaminergic system. Additionally Benjamin and Epstein provide support of the earlier work of Susan George and associates at the University of Toronto who found a strong association between the D4 gene variance and alcoholism and nicotine dependence again showing the interchangeable nature of this syndrome. Therefore, this is another element in the “To Binge or Not To Binge?” equation.

Many genes have been identified that may play a role in increasing susceptibility to obesity. Reduced dopamine function appears to play a role in dysfunctional eating patterns and may predispose some individuals to obesity. The long version of the D4 dopamine receptor gene (D4DR) has been shown to alter receptor function and reduce intracellular response to dopamine. It also has been associated with novelty-seeking-related personality traits that are found with greater frequency in obese individuals. Poston and associates examined the association between the D4DR and obesity in 115 obese patients participating in a weight management program. They constructed four models of increased obesity that included combinations of traditional risk factors (i.e. history of obesity, parental obesity, a body mass index >40) in elevations on the novelty scales of the Karolinska Scales of Personality. There was a significant increase in the frequency of the D4DR long alleles in individuals defined as high risk using the combination of novelty-seeking-related personality traits, severe obesity (i.e. BMI >40), and any other traditional risk factor, but not with the traditional risk factors alone. These preliminary data suggest a potential role for the DR4D gene in increasing obesity susceptibility.

There are known limitations which will be addressed:
Competition for uptake of precursors across the blood brain barrier—While it is well known that the ability of phenylalanine, tyrosine and tryptophan to penetrate the blood brain barrier is mediated by a shared active transport system, it is also well known that the use of Chromium could significantly assist in enhancing or concentrating blood tryptophan into the brain by its effect on insulin release and subsequent enhance glucose utilization by increase glucose receptor sensitivity. Along with this effect is the effect glucose has on increasing muscle absorption of large amino acids like leucine, valine and isomeric forms, into muscle (see below). This all works in concert to enhance transport of tryptophan into the brain and increase serotonin synthesis. This is the exact reason for the chromium inclusion at a dose of 1000 mcgs and the usual lower dosage of between 200-400 mcgs. Moreover, even if this was not effective to increase the synthesis of serotonin, with the formula it could bypass serotonin and work through its enkephalinase activity of D-phenylalanine. This by itself would cause dopamine release via its inhibition of SN GABA. In earlier work by Wurtman, he showed that by reducing blood glucose the brain will concentrate up to 33% more blood borne tryptophan. Furthermore, it is well known that if you reduce tryptophan levels in the hypothalamus you will reduce brain serotonin levels and its neuronal release. Conversely, elevating tissue tryptophan levels (could be accomplished by adding natural 5-hydroxytryptophan or chromium) increases both serotonin levels and serotonin release. According to Wurtman and associates, these observations demonstrate for the first time that both precursor-dependent elevations and reductions in brain serotonin levels produce proportionate changes in serotonin release, and the magnitude of the tryptophan effect is unrelated to neuronal firing frequency Suggesting the importance of precursor administration to increase serotonin levels. In essence, the data support the hypothesis that serotonin release is proportionate to intracellular serotonin levels.
Nutrient-Dependent Control Of Brain Catecholamine Synthesis & Release—While brain serotonin synthesis is affected by availability of tryptophan or 5-hydroxytryptophan under control conditions, precursor dependency of catecholamine synthesis in the brain is coupled to the firing rate of the tyrosine hydroxylase (TOH) containing neuron. It has been demonstrated in a number of studies that a supplementation of l-tyrosine does not augment the synthesis of catecholamines under resting non-stressed condition, while an enhanced neuronal activity will increase the synthesis and release of catecholamines, especially dopamine following tyrosine application. Of these physiological stress is the most important, in terms of enhancing neuronal activity. In essence stress is mandatory for l-tyrosine administration to affect catecholamine synthesis. While it is well known research has demonstrated that catecholamines such as norepinephrine and dopamine can act as feedback inhibitors of tyrosine hydroxylase, the enzyme that converts tyrosine into the immediate precursor for dopamine or norepinephrine, under physiological stress this mechanism is obliterated. The reason for this is that the mechanisms that couple catecholaminergic neuron's firing frequency to its precursor responsiveness involve the phosphorylation of the TOH enzyme portion. This enzyme's affinity for its cofactor is thereby enhanced and it becomes independent of end-product inhibition, yet dependent on the availability of its precursor substrate. In essence, under stress l-tyrosine supplementation becomes similar to the l-tryptophan (5-hydroxytryptophan) type of precursor responsiveness. Moreover, it is important to realize that this enzyme activation occurs under enhanced neuronal activity and is calcium—and calmodulin dependent. In addition, the phosphorylation of TOH can be catalyzed by several protein kinases that selectively act on different amino acids of the enzyme protein. The protein kinase enhances the enzyme activity without affecting end-product inhibition or the affinity to tyrosine or the tetrahydrobiopterin cofactor. In addition, it is well known that a different, cAMP-dependent protein kinase can also phosphorylate tyrosine hydroxylase, enhancing the affinity to the cofactor (but not to tyrosine) and reducing the regulation by end-product inhibition. In summary, these changes allow the enzyme activity to become dependent on the extent to which it is saturated with its substrate l-tyrosine.

Treatment: Role of “Nutrients and Pharmacogenomics in Obesity and Overeating—In the eating game we must first appreciate the importance of brain neurochemistry and how certain nutrients such as amino-acids could effect brain neurotransmitter status and how this could effect macro-selection. In this regard we must be cognizant of how a nutritionally unbalanced diet may lead to neurochemical processes that now induce the intake and aberrant craving of high carbohydrate meals. The intake of macro- and micronutrients leads to characteristic changes in the serum concentration of amino acids, in particular large neutral amino acids. The consensus of the literature suggests that changes in the concentration of large neutral amino acids lead to parallel changes in their brain concentration that, in turn, specifically influence the synthesis of their respective neurotransmitters.

While the functional impact of these neurotransmitters differs markedly, the basic metabolic processes are comparable. Most of these substances are metabolized within nerve cells from their precursor molecules that have been taken up from the extracellular brain fluid. They are stored in intraneuronal vesicles and are released following a depolarization of the neuron. They interact with either pre-or postsynaptic receptors within the synaptic cleft and ere inactivated either through enzymatic degradation or through neuronal uptake. Central nervous system functions clearly depend on those mechanisms that guarantee the stability of precursor amino acid concentration. It also follows that a marked reduction in the concentration of these amino acids impairs physiological functions that are regulated and/or modulated by a respective neurotransmitter. The regulation of the synthesis of metabolic products from large neutral amino acids appears to be specific for neurotransmitters such as monoamines. It is noteworthy that a similar impact on the synthesis of neurohormones (i.e. opioid peptides) does not exist, since ribosomal protein synthesis does not depend on the fluctuation of amino acid concentrations. It may thus be speculated that a coupling of nutrient intake (amino-acid precursors), transmitter synthesis, and neuronal function reflects a phylogenetically relevant process.

When we consider that there is shared genes and RDS is an encompassing term which includes a number of impulsive, compulsive and addictive behaviors, we should not be surprised of the vast numbers involved in RDS. We know that at least one-third of the US alone carries the DRD2 A1 variant. This has been linked to multiple addictions including carbohydrate bingeing and other drugs of abuse (i.e. alcohol, cocaine, nicotine) and its presence at birth predicts future problems with food , drugs and certain destructive behaviors at the predictive value of 74%.

While we believe natural nutritional therapy could offer an important approach to prevent as well as treat reward deficit problems, especially as it relates to obesity, there is reason to believe a pharmacological approach cannot be ignored. In an attempt to show the power of a new emerging field of “Nutragenomics” we provide the following example.

It is tempting to speculate that the pharmacological sensitivity of overeaters to dopaminergic agonists (bromocriptine, bupropion, n-propylnor-apomorphine, phentermine and dopamine) may be determined partly by the individuals D2 genotype. We predict that A1 carriers should be more responsive to D2 agonists (including naturally released dopamine), especially in stimulant-dependent people. At least one study already has shown that direct microinjection of the D2 agonist n-propylnor-apomorphine into the rat nucleus accumbens significantly suppresses the animals symptoms after withdrawal of opiates. A double-blind study demonstrates the utility of this approach in human subjects. The D2 agonist bromocriptine or a placebo was administered to alcoholics who were carriers of the A1 allele (A1/A1 and A1/A2 genotypes) or who carried only the A2 allele (A2/A2). The greatest improvement in the reduction of craving and anxiety was found among the A1 carriers who were treated with bromocriptine. The attrition rate (relapse) was highest among the A1 carriers who were treated with the placebo. It is noteworthy, that as expected, dopamine receptor occupancy by a dopamine agonist or by dopamine itself, initiates a feedback system that produces more dopamine receptors even in A1 carriers (low dopamine receptors) after a period of time. This is supported by the fact that the greatest effect occurred after a period of six weeks. In support of this, since 1993, Molinoff and associates using transfected kidney cells, consistently showed that occupancy of D2 receptors by dopamine agonists over time results in proliferation of dopamine D2 receptors.

To reiterate, Blum and associates found similar evidence with chromium picolinate, for the role of genes in physiological response with nutritional supplements. While there still is controversy regarding the effects of chromium salts (picolinate and nicotinate) on body composition and weight loss in general, recent unpublished work seems to support the positive change in body composition in humans. In consideration of the above study, Chen and Blum and others decided to genotype 130 overweight subjects for the dopamine D2 receptor gene. The subjects were assessed for scale weight and for percent body fat using dual energy X-ray absorptionmetry (DEXA R). The subjects were divided into matched placebo and chromium picolinate groups (400 μg. per day). The sample was separated into two independent groups; those with either an A1/A1 or A1/A2 allele and those with only the A2/A2 pattern. In the A2/A2 carriers, the measures of change in fat weight, change in body weight, the percent change in weight, and the body weight change in kilograms were all significant, whereas no significance was found for any parameter for those subjects possessing a dopamine D2 receptor A1 allele. These results suggest the dopaminergic system, specifically the density of the D2 receptors, confers a significant differential therapeutic effect of chromium picolinate in terms of weight loss and change in body fat. Moreover, we propose for the first time that mixed effects now observed with chromium picolinate in terms of body composition, may be resolved by typing the patient via dopamine D2 receptor genotyping prior to treatment with not only chromium salts, but with other nutritional supplements as well.

Brain Nutrition and Behavior—A detailed account of this subject is treated in the books Alcohol and The Addictive Brain (Blum, 1991 The Free Press), and To Binge or Not to Binge?(Blum, Cull & Miller, 1998 Psychiatric Genetic Press). In short, if genetic anomalies result in neurotransmitter imbalance, then how could we help to restore balance? At the functional level, it seems clear that neurotransmitter imbalance may be a problem of brain nutrition: more specifically, a deficiency or excess of amino acids. In the healthy body given adequately nourishment, amino acids are in balance; if there is an excess or shortage, distortions of brain function can result.

As we know the brain cannot synthesize all of the amino acids involved in the formation of neurotransmitters; some are derived from food metabolism, and come to the brain via the blood supply. There are two categories of amino acids: essential and nonessential. There are five essential amino acids necessary for the manufacture of neurotransmitters, thought to play a role in obesity: methionine, leucine, phenylalanine, tyrosine, and tryptophan (see above for more detail). Among the nonessential amino acids manufactured in the body, Glutamine probably plays a significant role, because it is involved in the manufacture of GABA. Two forms of amino acids are found in nature. The amino acids in the brain that make up the neurotransmitters, and the enzymes that regulate them, are all derived from the L-form. The D-form (as in D-phenylalanine) is found in a few microorganisms and in multi-cellular organisms like frog skin.

Single Versus Multiple Amino acid Macronutrients

First, although a single amino acid may be involved in the formation of a given neurotransmitter, it does not act alone. It needs the help of co-factors such as vitamins and minerals before the formation can take place. For example, vitamin B6 (in the alcoholic, pyridoxal-5-phosphae form is required) is needed for the manufacture of dopamine.
Second, obesity is the result of a complex disorder that involves processes taking place in the neuron, at the synapse, and at receptors.
Third, we cannot determine (until we use DNA tests) the specific defect that is producing a particular part of the problem. Therefore, in the effort to offset neurotransmitter deficits, it is not feasible to depend on single amino acids. This is why we include both serotinergic and dopaminergic precursors.
Fourth, an odd characteristic of the blood/brain barrier actually makes treatment easier.

Most overweight individuals have compounded stress and may have comorbid addictions like alcohol, smoking, and other drugs; it is known that all of these weaken the barrier facilitating the passage of restorative substances such as amino acids into the brain. This is particular important when you consider large neutral amino carrier system and competition of tryptophan, phenylalanine and tyrosine. It is equally important when you consider, as mentioned earlier, that the rate limiting enzyme Tyrosine Hydroxylase works best under stressful conditions and the precursor tyrosine will indeed be converted to dopamine and will be subsequently released into the synapse of the N. accumbens.
Fifth, it is well known that the degradation of catecholamines by COMT plays a role, albeit only partial, in clearing these neurotransmitters from synaptic cleft. Dopamine, norepinephrine and serotonin reuptake into nerve terminals via membrane transporter is thought to play a more significant role. However, it is our position that any enhancement of the neurotransmitters in the synapse is positive. In this regard, the effects of synephrine on norepinephrine receptors plus the central nervous system effects of Rhodiola rosea could contribute to a sibutramine/d-fenfluamine-like effect. The amount of Rhodiola rosea recommended in the formula is 240 mg per day (based on a 3% extract standardized to rosavin) which is somewhat higher than the recommended dose for use of Rhodiola rosea as an antidepressant (200 mg/day). Moreover, the NGI formula also contains synephrine, derived from citrus aurantium (6% synephrine) at a daily dose of 50 mg. This amounts to only 6 mg per day. While this is less than what is normally recommended as s sympathomimetic agent, when combined with caffeine thermogenesis could be achieved without the stimulatory effects seen with much higher doses (104 mg/day).

Studies Showing Anti-craving Efficacy of Precursor Amino-acids and Enkephalinase Inhibitor Activity—It is our contention that with the formula as designed for anti-craving, additive or even synergistic outcomes might be observed since the ingredients are included that could act through several different mechanisms to enhance the activity of the neurotransmitters. The patented complex has been named Synaptamine.
In a number of experiments we have shown brain changes of the enkephalins using d-phenylalanine (500 mg/kg/day for 18 days and or its metabolite hydrocunnamic acid (intracerebral ventricular injection of 25 micrograms) in mice; Using the same doses these known enkephalinase inhibitors significantly reduced alcohol preference in both acceptance and 14 day preference test.
We have shown in healthy volunteers electrophysiological changes (enhanced memory and focus) with the combination of DL-phenylalanine (1500 mg/day), L-tyrosine (900 mg/day) , L-glutamine (300 mg/day) , chromium picolinate (360 micrograms/day) and other co-factors;
Positive effects in alcoholics in an in-patient hospital including lower building up to drink scores, required no PRN benzodiazepines, (0% vs. 94%), ceased tremoring at 72 hours, had no severe depression on the MMPI, in contrast to 245 of control group (Blum et al. 1988). The ingredients included Dl-phenylalanine(2760 mg/kg/day), L-tryptophan (150 mg/day), L-glutamine (150 mg/day), and pyridoxal-5-phosphate (30 mg/day);
In a double-blind placebo controlled study of polysubstance abusers in an in-patient hospital, the combination of Dl-phenylalanine (2760 mg/day), L-tryptophan (150 mg/day), L-glutamine (150 mg/day), and pyridoxal-5-phosphate (30 mg/day), significantly reduced stress, improved physical and emotional scores, a six-fold reduction in AMA rates, enhanced treatment recovery;
Utilizing DL-phenylalanine (1500 mg/day), L-tyrosine(900 mg/day), L-glutamine (300 mg/day), L-tryptophan (400 mg/day) and pyridoxal-phosphate (20 mg/day) in inpatient treatment of cocaine abusers over a 30 day period compared to controls significantly reduced drug hunger and withdrawal against advice rate (AMA), reduced need for benzodiazepines, and facilitated retention in the treatment program;
In an outpatient clinic DUI offenders (alcoholics and/or cocaine addicts) were treated with a combination of dl-phenylalanine, L-tyrosine, L-glutamine, Chromium, pyidoxyl-5-phosphate over a ten month period. Compared to a vitamin control (only B-complex and vitamin c), the experimental group significantly reduced relapse rates and enhanced recovery in these DUI outpatient offenders. The retention rates obtained for alcoholics was 87% for the experimental group compared to only 47% of the control patients and for cocaine abusers the numbers are 80% vs. only 13%. For alcoholics: DL-phenylalanine (2760 mg/day), L-Glutamine (150 mg/day), chromium picolinate (360 micrograms/day), pyridoxal-5-phosphate; For cocaine abusers: DL-phenylalanine ( 1500 mg/day) , L-Tyrosine (900 mg/day), L-glutamine (300 mg/day), pyridoxal-5-phosphate (20 mg/day).
Utilizing amino-acid and enkephalinase inhibitory therapy, J. A. Cold found significant improvement in both cocaine craving and withdrawal symptoms in out patient cocaine addicts. The ingredients included DL-phenylalanine (1500 mg/day), L-Tyrosine (900 mg/day), L-glutamine (300 mg/day), pyridoxal-5-phosphare (20 mg/day).
With only chromium picolinate it was found in two double-blind placebo controlled studies that doses of either 00 mcg or 400 mcg resulted in a body composition improvement, loss of body fat, gain in nonfat mass;
In addition see above for similar results dependent on the DRD2 A1 variant (unpublished Blum & Kaats);
With DL-phenylalanine (2700 mg/day), L-tryptophan (150 mg/day), L-glutamine (150 mg/day) and pyridoxal-5 phosphate (30 mg/day) it was also found that 27 outpatients with high carbohydrate bingeing behavior where females were assigned 800 calories total intake per day and males were assigned 1,000 to 1,200 calories per day and all withdrew from sugar use attending a supervised diet-controlled treatment program, the supplement group over a 90 day period lost an average of 26.96 pounds compared to the control group (no supplement) lost only 10 pounds. In fact, only 18.2% of the experimental group relapsed (lost less than 15 pounds over the 90 day period) compared to 8.% in the control group;
In another study where the supplement contained dl-phenylalanine (2760 mg/day), L-tryptophan (150 mg/day), L-glutamine (150 mg/day), pyridoxal-5 phosphate (30 mg/day), chromium Picolinate (200 micrograms/day), and camitine (60 mg/day) over a 2-year period in 247 obese patients the following results were obtained in a dual blind non-randomized open trial utilizing Centrum vitamin as a control: compared with the Non-PhenCal/Centrum group the experimental PhenCal/Centrum group showed a two-fold decrease in percent overweight for both males and females; a 70% decrease in food cravings for females and a 63% decrease for males; and a 66% decrease in binge eating for females and a 41% decrease for males. Most importantly, the experimental group regained only 14.7% of the lost weight, and multiple regression modeling revealed that with PhenCal treatment, morbid obesity and binge eating score were significant predictors of weight gain after 2 years. In contrast, family history of chemical dependence was most closely associated, although not statistically significant, with improved results with PhenCal.
Blum decided to test the hypothesis that possibly by combining a narcotic antagonist and amino acid therapy consisting of an enkephalinase inhibitor (D-Phenylalanine) and neurotransmitter precursors (L-amino acids) to promote neuronal dopamine release might enhance compliance in methadone patients rapidly detoxified with the narcotic antagonist Trexan® (DuPont, Del.). In this regard, Thanos et. al. and associates found increases in the dopamine D2 receptors (DRD2 ) via adenoviral vector delivery of the DRD2 gene into the nucleus accumbens, significantly reduced both ethanol preference (43%) and alcohol intake (64%) of ethanol preferring rats, which recovered as the DRD2, returned to baseline levels. This DRD2 over expression similarly produced significant reductions in ethanol non-preferring rats, in both alcohol preference (16%) and alcohol intake (75%). This work further suggests that high levels of DRD2 may be protective against alcohol abuse. The DRD2 A1 allele has also been shown to associate with heroin addicts in a number of studies. In addition, other dopaminergic receptor gene polymorphisms have also associated with opioid dependence. For example, Kotler et al. showed that the 7 repeat allele of the DRD4 receptor is significantly over presented in the opioid dependent cohort and confers a relative risk of 2.46. This has been confirmed by Li et. al. for both the 5 and 7 repeat alleles in Han Chinese case control sample of heroin addicts. Similarly Duaux et. al. in French Heroin addicts, found a significant association with homozygotes alleles of the DRD3-Bal 1. A study from NIAAA, provided evidence that strongly suggests that DRD2 is a susceptibility gene for substance abusers across multiple populations. Moreover, there are a number of studies utilizing amino-acid and enkephalinase inhibition therapy showing reduction of alcohol, opiate, cocaine and sugar craving behavior in human trials. Over the last decade, a new rapid method to detoxify either methadone or heroin addicts utilizing TrexanR sparked interest in many treatment centers throughout the United States, Canada, as well as many countries on a worldwide basis. In using the combination of TrexanR and amino-acids, results were dramatic in terms of significantly enhancing compliance to continue taking Trexan®. The average number of days of compliance calculated on 1,000 patients, without amino-acid therapy, using this rapid detoxification method is only 37 days. In contrast, the 12 subjects tested, receiving both the Trexan® and amino-acid therapy was relapse-free or reported taking the combination for an average of 262 days (P<0.0001). Thus coupling amino-acid therapy and enkephalinase inhibition while blocking the delta receptors with a pure narcotic antagonist may be quite promising as a novel method to induce rapid detox in chronic methadone patients. This may also have important ramifications in the treatment of both opiate and alcohol dependent individuals, especially as a relapse prevention tool. It may also be interesting too further test this hypothesis with the sublingual combination of the partial opiate mu receptor agonist buprenorphrine. The ingredients tested included DL-phenylalanine (2760 mg/day), L-Glutamine (150 mg/day), chromium picolinate (360 micrograms/day), pyridoxal-5-phosphate (30 mg/day).
Most recently a study was performed by Julia Ross best selling author of The Diet Cure (Viking Press USA, 1999; Penguin UK, Au, and USA, 2000), in an outpatient clinic in Mill Valley, Calif. involving amino-acid therapy and enkephalinase inhibition based on Blum's work. At Recovery Systems, Ross has successfully utilized this approach to treat a number of RDS behaviors, especially eating disorders. In a preliminary evaluation, utilizing the following ingredients tailored made for each client, dl-phenylalanine, 5-hydroxytryptophan, l-tryptophan, l-tyrosine, l-glutamine, chromium, vitamin B6, follow-up interviews of six randomly selected former eating disordered female clients (three were also chemically dependent), were contracted nine months to three years post-treatment to evaluate efficacy of combining targeted nutritional elements (amino-acids, vitamins, digestive enzymes, a diet low in refined carbohydrates but adequate in calories and other nutrients) with conventional counseling, education, and peer support. Follow-up confirmed significant initial benefits in mood and freedom from compulsive behavior and ideation in 100% tested. While one subject relapsed within six months, the remaining five subjects all sustained, and in some cases exceeded expectations. Following this preliminary evaluation, the authors also evaluated an additional 100 patients and the data collected revealed 98% significant improvement in both mood and reduced craving for not only carbohydrates but other abusable substances as well. According to Ross this work further suggests the positive potential of adding targeted nutritional protocols to conventional treatment elements to improve outcome in an RDS intransigent population.
A study in Las Vegas at an outpatient clinic has been completed. The following results have been evaluated and presented herein. Relapse rates: CCD:—Out of 15 patients only 2 patients dropped out, while the other 13 patients remained in the program for 12 months. Therefore, the percent relapse for this group is13.33; CC—Out of 43 patients 11 patients dropped out, while the other 32 patients remained in the program for 12 months. Therefore, the percent relapse for this group is 23.2.; FCS—Out of 10 patients only 2 dropped out, while the other 8 patients remained in the program for 12 months. Therefore, the percent relapse for this group is 20.0.; SR—Out of 8 patients none dropped out, thus 8 patients remained in the program for 12 months. Therefore, the percent relapse for this group is 0.0. If we calculate the percent relapse of the entire program which included a total of 76 patients with a total of 15 patients that dropped out it is a remarkable 19.9% relapse. The majority of drop outs (11 out of 15 or 73.3%) were methamphetamine abusers—the ingredients include DL-phenylalanine (2700 mg/day), 5-hydroxytryptophan (20 mg/day), L-Tyrosine (750 mg/day), L-glutamine (350 mg/day), Rhodiola rosea (3% rosavin) (66 mg/day), Chromium dinicotinate glycerate 1000 micrograms/day), DMAE (40 mg/day), Huperzine A (150 micrograms/day). Combination of vitamins (C,E, Niacin, Riboflavin, Thiamin, B6 [20% Pyridoxal-5 phosphate and 80% Pyridoxine],folic acid, B12, Biotin, Pantothenic acid, Calcium, Magnesium, zinc, Manganese and a herbal calming blend, focus blend or mood enhancing blend. The ingredients and dosage was dependent on type of abusers including diagnosis of ADHD.

Fortunately, if a broad menu of amino acids is available in sufficient quantity, the brain appears to have the ability to choose from the menu the one or ones needed to manufacture more of the neurotransmitter that is deficient. Based on the patents and technology afforded to us, the following nutrients are scientifically formulated and have been clinically tested for over 20 years and have relevance to the problem defined as “Reward Deficiency Syndrome”, more specifically-overeating and carbohydrate bingeing. However, the work to date supports a generalized anti-craving claim.
D-Phenylalanine, to inhibit enkephalinase, the enzyme that metabolizes or breakdown enkephalins, thereby increasing the availability of enkephalins and, presumably, making more dopamine available at the reward sites especially under stressful conditions.
L-Phenylalanine, to stimulate the production of dopamine, and/or increase norepinephrine levels in the reward area of the brain. The major problem with this amino acid is that it could compete with other amino acids, such as blood borne l-tryptophan and l-tyrosine at the large neutral amino-acid brain carrier system (see Milner et al. 1986). However, other data demonstrates for the first time that the synthesis and release responses to some dopaminergic agents may be elicited from synaptosomal dopamine, which is formed by the hydroxylation of phenylalanine. Amphetamine and Cogentin increased the release of dopamine formed from 14C-phenylalanine in rat caudate nucleus synaptosomal preparation and concomitantly stimulated the synthesis. Amfoelic acid also caused a net release of that dopamine. In conclusion, the results suggest that synaptosomal particles represent a unit capable of synthesizing dopamine from l-phenylalanine and that synthesis from this precursor may be under the regulatory control of the particles.
L-glutamine, to increase brain GABA levels at receptors associated with anxiety. Its major use is to maintain balance in case of over inhibition by D-phenylalanine.
L-5-hydroxytryptophan (or its natural form)—The effect of systemic administration of 5-hydroxy-l-tryptophan on the release of serotonin in the lateral hypothalamus of the rat in vivo as examined utilizing brain microdialysis. Administration of 5-HTP caused an immediate increase of the 5-HT in dialysates, which was long lasting and dose dependent. When calcium was omitted from the perfusion medium, thereby limiting exocytosis, levels of basal 5-HT were significantly decreased and the 5-HTP-induced response of 5-HT was markedly attenuated.
Pyridoxal-5-phosphate, the active ingredient of vitamin B6 to serve as a co-factor in the production of neurotransmitters and to enhance the gastrointestinal absorption of amino acids.
Chromium Salts (Nicotinate and Picolinate), have a number of metabolic effects including: increase of insulin sensitivity; reduction of cholesterol; reduction of percent body fat; reduction of weight loss; maintaining muscle mass promoting lean; enhancing body composition; promotes brain serotonin production (see above).
Calcium, promotes neurotransmitter release based on many studies.
Rhodiola rosea—Several clinical trials with double-blind placebo controls in Russia provide evidence that R. rosea possess positive mood enhancing and anti-stress properties with no detectable levels of toxicity. Generally, R. rosea extract has been shown to have a positive influence on the higher nervous system, increasing attention span, memory, strength and mobility of the human body, and weight management. It is believed that R. rosea can act as a COMT inhibitor where brain levels of serotonin and dopamine has been observed. Studies by Saratikov and Marina suggest that R. rosea can increase the level of neurotransmitters by 30 percent and decrease COMT activity by 60 percent. In the weight management area there are double-blind studies with regard to weight loss and fat mobilization.

DIABETES and OBESITY—Banaba and Glucose Transport—Obesity is a major risk factor for Syndrome X and type 11 diabetes (T2D). However, most antidiabetic drugs that are hypoglycemic also promote weight gain, this alleviating one symptom of T2D while aggravating a major risk factor that leads to T2D. Adipogenesis, the differentiation and proliferation of adipocytes, is a major mechanism leading to weight gain and obesity. It is highly desirable to develop pharmaceuticals/nutraceuticals and treatments for T2D that reduce blood glucose levels without inducing adipogenesis in patients. There have been reports that an extract from Lagersremia speciosa L. (banaba) possessed activities that both stimulated glucose transport and inhibited adipocyte differentiation in 3T3-L1 cells. It turns out that the major effect of Banaba is due to Tannic Acid. Moreover, Tannic Acid induces phosphorylation of the insulin receptor (IR), as well as translocation of the glucose transporter (GLUT 4), the protein factors involved in the signaling pathway of insulin-mediated glucose transport. Tannic Acid has been also found to inhibit the expression of key genes for adipogenesis. The following genes are inhibited by Tannic Acid: PPAR-gamma 2; c-fos; c-jun and c-myc. Tannins, as plant-derived long-chain polyphenolic compounds, are part of our daily diet. Tannic Acid was previously shown to be antilipogenic in an animal study. Tannic Acid in earlier studies was shown to be anti-diabetic in humans. The combination of the 2 activities of Tannic Acid makes it ideally suited as a prototypic compound to treat Syndrome X and T2D effecting hyperglycemia, hyperinsulinemia, hypertriglyceridemia, without concomitant weight gain or even with weight loss.

Cinnamon and Diabetes—Cinnamon significantly reduces blood sugar levels in diabetics, a new study has found. The discovery was initially made by accident, by Richard Anderson at the US Department of Agriculture's Human Nutrition Research Center in Beltsville, Md. “We were looking at the effects of common foods on blood sugar,” he told New Scientist. One was the American favorite, apple pie, which is usually spiced with cinnamon. “We expected it to be bad. But it helped,” he says. Sugars and starches in food are broken down into glucose, which then circulates in the blood. The hormone insulin makes cells take in the glucose, to be used for energy or made into fat. But people with Type 1 diabetes do not produce enough insulin. Those with Type 2 diabetes produce it, but have lost sensitivity to it. Even apparently healthy people, especially if they are overweight, sedentary or over 25, lose sensitivity to insulin. Having too much glucose in the blood can cause serious long-term damage to eyes, kidneys, nerves and other organs. Molecular Mimic—The active ingredient in cinnamon turned out to be a water-soluble polyphenol compound called MHCP. In test tube experiments, MHCP mimics insulin, activates its receptor, and works synergistically with insulin in cells. To see if it would work in people, Alam Khan, who was a postdoctoral fellow in Anderson's lab, organized a study in Pakistan. Volunteers with Type 2 diabetes were given one, three or six grams of cinnamon powder a day, in capsules after meals. All responded within weeks, with blood sugar levels that were on average 20 per cent lower than a control group. Some even achieved normal blood sugar levels. Tellingly, blood sugar started creeping up again after the diabetics stopped taking cinnamon. The cinnamon has additional benefits. In the volunteers, it lowered blood levels of fats and “bad” cholesterol, which are also partly controlled by insulin. And in test tube experiments it neutralized free radicals, damaging chemicals, which are elevated in diabetics. Cinnamon Helps Type 2 Diabetes—Also Helps Cholesterol—Cinnamon can improve glucose and cholesterol levels in the blood. For people with type 2 diabetes, and those fighting high cholesterol, it's important information. Researchers have long speculated that foods, especially spices, could help treat diabetes. In lab studies, cinnamon, cloves, bay leaves, and turmeric have all shown promise in enhancing insulin's action, writes researcher Alam Khan, PhD, with the NWFP Agricultural University in Peshawar, Pakistan. His study appears in the December issue of Diabetes Care. Botanicals such as cinnamon can improve glucose metabolism and the overall condition of individuals with diabetes—improving cholesterol metabolism, removing artery-damaging free radicals from the blood, and improving function of small blood vessels, he explains. Onions, garlic, Korean ginseng, and flaxseed have the same effect. In fact, studies with rabbits and rats show that fenugreek, curry, mustard seeds, and coriander have cholesterol-improving effects. But this is the first study to actually pin down the effects of cinnamon, writes Kahn. Studies have shown that cinnamon extracts can increase glucose metabolism, triggering insulin release—which also affects cholesterol metabolism. Researchers speculated that cinnamon might improve both cholesterol and glucose. And it did! The 60 men and women in Khan's study had a diagnosis of type 2 diabetes for an average of 6 1-2 years but were not yet taking insulin. The participants in his study had been on anti-diabetic drugs that cause an increase in the release of insulin. Each took either wheat-flour placebo capsules or 500 milligram cinnamon capsules.
Group 1 took 1 gram (two capsules equaling about one-quarter of a teaspoon) for 20 days.
Group 2 took 3 grams (six capsules, equaling a little less than one teaspoon) for 20 days.
Group 3 took 6 grams (twelve capsules, equaling about one and three-quarters teaspoons) for 20 days.

Blood samples were taken at each level of the study. Cinnamon made a difference! Twenty days after the cinnamon was stopped, there were significant reductions in blood glucose levels in all three groups that took cinnamon, ranging from 18 to 29%. But these were one peculiar finding that researchers don't understand at this point. Only the group that consumed the lowest level of cinnamon continued with significantly improved glucose levels—group 1. The placebo groups didn't get any significant differences. Taking more cinnamon seems to improve the blood levels of fats called triglycerides. All the patients had better triglyceride levels in their 40-day tests—between 23% to 30% reductions. Those taking the most cinnamon had the best levels. In groups taking cinnamon pills, blood cholesterol levels also went down, ranging from 13% to 26%; LDL cholesterol also known as “bad” cholesterol went down by 10% to 24% in only the 3- and 6-gram groups after 40 days. Effects on HDL (“good cholesterol”) were minor.

Desnutrin—A new member of a family of proteins functioning in the regulation of lipolysis in adipose tissue has been discovered and named “Desnutrin.” This substance is transiently induced by fasting and decreased by re-feeding. A close homolog, termed adiponutrin, has the opposite expression pattern, being induced by feeding and disappearing upon fasting. Desnutrin functions by acting as the first enzyme in lipolysis, hydrolyzing triglycerides to diglycerides, whereas the well-known hormone-sensitive lipase takes the diglycerides to monoglycerides and on to fatty acids. When demand increases, adipose lipolysis is stimulated sparing glucose for brain function. It has been proposed by Vilene et. al. (2004), that the function of Desnutrin was the lipolysis of triglycerides stored in adipose tissue to provide FFA for supply of energy during fasting. The basic mechanism of fat mobilization can be schematized as follows:

Where TG=triglycerides; DG=diglycerides; Mg=monoglycerides, HSL=hormone sensitive lipase; 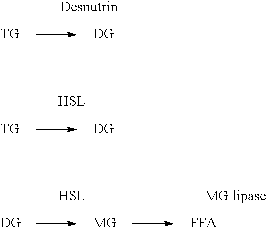
The lipolytic cascade:
TG→DG→MG→FFA

In terms of energy production the genes involved include: Sterol Regulatory Element Protein-1 (SREBP-1c ); mitochondrial glycerol-3-phosphate acyltransferase gene (MGPAT) and the peroxisome proliferator—activated receptor (PPAR—gamma-2). The genes that regulate both Desnutrin and adiponitrin may be important candidate genes for energy regulation in the adipose cell.

Tryptophan 2,3-Dioxygenase (TDO2) Glucocorticoid Response—Like Element—Abnormalities in serotonin levels have been implicated on a wide range of psychiatric disorders. tryptophan 2,3-dioxygenase is the rate-limiting enzyme in the catabolism of tryptophan, the precursor of serotonin. As such it is a major candidate gene in psychiatric genetics. The regulatory, intron and exon regions of the human TDO2 gene have been sequenced and 12 exons were identified. Two polymorphisms consisting of G→T and G→A mutations 2 bp apart in intron 6 have been identified. The 3′ end of intron 5 showed an extensive CCCCT pentanucleotide repeat that was markedly polymorphic. The TDO2 gene regulatory region has an insertion of approximately 1064 bp of random DNA beginning at −293 bp and extending to −1357 bp. This displaced the glucocorticoid response element (GRE) occurring at −1174 bp in the rat to −1500 in the human. In the human, within the DNA insert there was a gRE-loke microsatellite region containing multiple GTT repeats plus additional (GT9n) sequences. The RDo2 gene is localized chromosome 4q31. This gene may have important ramifications with regard to effects of serotinergic induction of food craving behavior.

High-Energy Diet and Genes—Obesity is an escalating problem in Western societies. It is possible that an individual's immediate and/or sustained appetite for apparently palatable foods, or metabolic adaptations to a new diet could be important. In rats, exposure to a high energy diet for 14 days resulted in a 7.7 g increase in bodyweight with increased caloric intake. Terminal levels of leptin, insulin, glucose, and non-esterified fatty acids (NEFAs) were all increased in these high energy fed diet animals. Moreover, appetite suppressant substances such as cocaine and amphetamine have transcription genes. Cocaine and amphetamine-regulated transcript (CART) and Mc4R gene expression in the hypothalamus were increased on a high energy diet. The animals passively over consume calories as a result of consuming a similar weight of a more energy dense food. This evokes physiological responses, which adjust caloric intake over several days. Circulating NEFA and insulin concentration, TCP-1, Mc4R and CART gene expression are increased as an immediate consequence of consuming a high energy diet, and may be involved in counting hypercaloric intake.

Echinacea and Immunomodulatory Genes—In vitro exposure of THP-1 cells to Echinacea species extracts induced expression (up to 10 fold) of the interleukin-1 alpha, interleukin-1 beta, tumor necrosis factor-alpha, intracellular adhesion molecule, interleukin-8, and interleukin-10 genes. This finding is consistent with a general immune response and activation of the nonspecific immune response cytokines. The overall gene expression pattern at 48 hr to 12 days after taking Echinacea was also consistent with an anti-inflammatory response. The expression of interleukin-1 beta, tumor necrosis factor-alpha, intracellular adhesion molecule, and interleukin-8 was decreased up through day 5, returning to baseline by day 12 the expression of interferon-alpha steadily rose through day 12, consistent with an antiviral response.

Analogy—Pharmacologic Mechanisms of the Drug Meridia: Comparison Proposed Anti-Craving Formula.

Meridia is an approved FDA drug for “weight loss” and weight management. The major effect of this drug is an anti-craving action derived from its effect to inhibit the reuptake of serotonin (5HT), dopamine (DA) and norepinephrine (NE). This inhibition of neurotransmitter reuptake results in an increase in the length of time 5HT, DA, and NE are available to act in the synaptic junction, and ultimately in an amplification of the neurotransmitter effects to reduce sugar/glucose cravings.

In its simplest form, the ingredients in the patented composition proposed for anti-craving effects mirrors the Meridia mechanism and should produce similar anti-craving effects. In this section we will point out the potential of the ingredients in the proposed formula, based on a large body of neurochemical evidence concerning precursor amino-acids; the role of chromium as a tryptophan enhancing substance; d-amino acid inhibition of enkephalinase; Rhodiola as a suspected inhibitor of catechol-O-methyl transferase (COMT) as well as Synephrine, a substance that can mimic some of the effects of catecholamines. Thus it is anticipated that since the same three neurotransmitters affected by Meridia (Sibutramine), could potentially be affected by certain ingredients, it should produce similar effects. It could be hypothesized that by increasing precursor (i.e. phenylalanine, tyrosine, and chromium and or 5-hydroxytryptophane or any other neurotransmitter enhancer even via transport) intake and inhibiting enzymatic degradation by COMT greater levels of 5HT, DA would be available at the synapse. The availability of the synapse is also increased since the D-phenylalanine causes preferential release of dopamine via opioid peptide breakdown inhibition. Thus the sum total effect is very much like Meridia and the following information will assure the scientific potential of this novel natural formula.

Most recently, Balcioglu and Wurtman, measured the effects of Sibutramine (Meridia), given intravenously, on brain dopamine and serotonin flux into striatal and hypothalamic dialysates of freely moving rats. While low doses of the drug had no effect, higher doses increased both serotonin and dopamine concentrations in the striatal and hypothalamic brain regions. These findings further support the neurochemical effects of Sibutramine, and suggest that the drug's anti-obesity action may result from changes it produces in brain dopamine as well as serotonin metabolism. The importance here is that it provides further support for the SYNAPTAMINE formula and both serotinergic and dopaminergic anti-obesity actions.

### SUMMARY

In essence, formulations of this type will cause the synthesis of the brain reward neurotransmitters like serotonin and catecholamines and through its effect on the natural opioids will by virtue of inhibiting GABA cause a significant release of dopamine at the nucleus accumbens. This constant release of possibly therapeutic amounts of dopamine (anti-stress substance) occupies dopamine D2 receptors, especially in carriers of the A1 allele (low D2 receptors and high glucose craving), and over time (possibly 6-8 weeks) effects RNA transcription leading to a proliferation of D2 receptors, thereby, reducing craving for aberrant substances, improving joint health and reducing the signs and symptoms of arthritis, reducing fat and optimizing, and providing anxiety relief.

### SUMMARY OF THE INVENTION

The present invention provides a business model and methods to measure genetic and metabolomic contributing factors affecting disease diagnosis, stratification, and prognosis, as well as the metabolism, efficacy and/or toxicity associated with specific vitamins, minerals, herbal supplements, homeopathic ingredients, and other ingredients for the purposes of customizing a subject's nutritional supplement formulation to optimize health outcomes.

The present invention provides a custom business model and methods to measure any genetic and metabolomic contributing factors affecting disease diagnosis, stratification, and prognosis, as well as the metabolism, efficacy and/or toxicity associated with specific vitamins, minerals, herbal supplements, homeopathic ingredients, and other ingredients for treating various health conditions including joint health involving reducing pain, inflammation, and joint damage; stress and anxiety relief; preventing sleep loss and insomnia; combating obesity and promoting weight loss; lethargy or lack of energy; skin, hair, and nail health; overall mental health and well-being; reducing the signs and symptoms of attention deficit hyperactivity disorder; reducing the signs and symptoms of depression; reducing the signs and symptoms of pre-menstrual dysphorric disorder; and, overcoming the dependence and urges of smoking, alcoholism, and drug dependence.

The present invention involves measuring multiple genetic mutations through single nucleotide polymorphisms, gene expression, or other forms of genetic and phenotypic measurement for the purposes of customizing or adjusting the formulation of nutritional supplements. Specifically, the present invention includes custom algorithms that combine genetic mutations into index values to represent specific pre-defined formulations.

The present invention applies to all genes currently discovered or which will be discovered and any nutritional or dietary supplement ingredient currently available or which will become available. The Salugen custom business model and methods, along with its algorithms are agnostic to gene and ingredient.

### DETAILED DESCRIPTION OF THE DRAWINGS

FIG. 1 is a diagram of the Brain Reward Cascade.

FIG. 2 is a diagram of the Reward Deficiency Syndrome and the DRD2 Dopamine Receptor.

FIG. 3 is a diagram of the brain mesolimbic system.

FIG. 4 is a flowchart of the neurotransmitter relations in the Brain Reward Cascade.

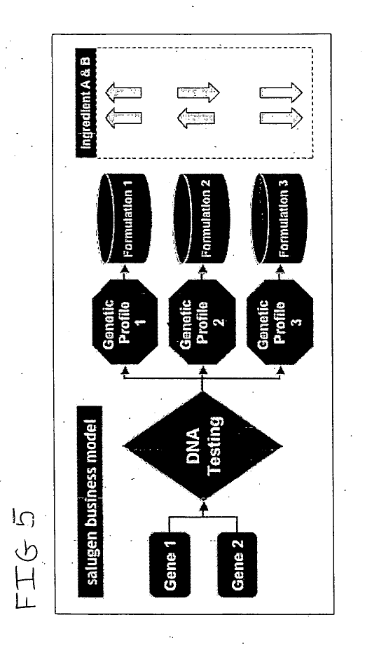

FIG. 5 is a diagram of the nutragenomics process to customize nutritional supplement formulations based upon DNA tests.

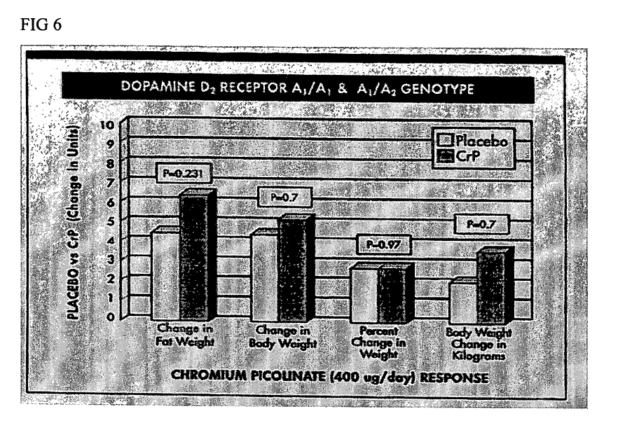

FIG. 6 is a chart illustrating how DRD2 A1 allele carriers do not respond to the positive effects of CRP.

### DETAILED DESCRIPTION OF THE INVENTION

The present invention provides a custom business model and methods to measure genetic and metabolomic contributing factors affecting disease diagnosis, stratification, and prognosis, as well as the metabolism, efficacy and/or toxicity associated with specific vitamins, minerals, herbal supplements, homeopathic ingredients, and other ingredients for the purposes of customizing a subject's nutritional supplement formulation to optimize health outcomes.

The present invention provides a custom business model and methods to measure any genetic and metabolomic contributing factors affecting disease diagnosis, stratification, and prognosis, as well as the metabolism, efficacy and/or toxicity associated with specific vitamins, minerals, herbal supplements, homeopathic ingredients, and other ingredients for treating various health conditions including joint health involving reducing pain, inflammation, and joint damage; stress and anxiety relief; preventing sleep loss and insomnia; combating obesity and promoting weight loss; lethargy or lack of energy; skin, hair, and nail health; overall mental health and well-being; reducing the signs and symptoms of attention deficit hyperactivity disorder; reducing the signs and symptoms of depression; reducing the signs and symptoms of pre-menstrual dysphorric disorder; and, overcoming the dependence and urges of smoking, alcoholism, and drug dependence.

The present invention involves measuring multiple genetic mutations through single nucleotide polymorphisms, gene expression, or other forms of genetic and phenotypic measurement for the purposes of customizing or adjusting the formulation of nutritional supplements. Specifically, the present invention includes custom algorithms that combine genetic mutations into index values to represent specific pre-defined formulations.

The present invention applies to all genes currently discovered or which will be discovered and any nutritional or dietary supplement ingredient currently available or which will become available. The Salugen custom business model and methods, along with its algorithms are agnostic to gene and ingredient.

### EXAMPLE

Custom Algorithm—For example, if a Salugen DNA test was measuring two genes through single nucleotide polymorphisms (Gene A and Gene B). The index scores that would be reported to the clinician and patient would be based upon the number of mutations. An index score of 0 would mean no mutation. An index score of 1 may mean a mutation in Gene A. An Index Score of 2 may mean a mutation in Gene B. An Index Score of 3 may mean a mutation in Gene A and Gene B, resulting in a simple report, easily understandable to both the clinician and patient that provides insights into disease diagnosis, stratification, prognosis, as well as the metabolism, efficacy and/or toxicity associated with specific vitamins, minerals, herbal supplements, homeopathic ingredients and other ingredients in nutritional or dietary supplementation. The index score is preferably called the GENOSCORE.

FIG. 5 conceptually illustrates the process of the present invention. Step 1 of the process involves collection of a DNA sample using a buccal swab, whole blood sample, or other accepted form of collection. Step 2 of the process involves measuring the specific genes using ELISA, TaqMan, PCR, Invader, or other technologies of measuring genetic and/or metabolomic activity, where available. Step 3 involves taking those measurements of genetic mutations and fitting them to a specific genetic profile (examples: Genetic Profile 1 is no gene mutations, Genetic Profile 2 is Gene 1 mutation, no mutation in Gene 2, and Genetic Profile 3 is Gene 1 mutation and/or Gene 2 mutation). Step 4 involves selecting the appropriate formulation based upon the genetic index or profile. Here is an example: If Genetic Profile 1, then Formulation 1 which has more of both Ingredients A and B. If Genetic Profile 2, then Formulation 2 which has more of Ingredient A but less of Ingredient B. If Genetic Profile 3, then Formulation 3 which has less of both Ingredient A and B.

Genoflex Joint Health Example

GFJH is an example of this unique nutragenomics process. To provide subjects with the greatest joint health and relief from pain and inflammation, we have assembled a formulation of clinically proven ingredients. The basic formulation is listed in the drawing below. This basic formulation represents an Index Score of 0, or no genetic mutations in the single nucleotide polymorphisms measured. Ingredients in Genoflex Joint Health have demonstrated in clinical studies to be as effective as ibuprofen in reducing joint damage, delaying progression and providing symptomatic relief from osteoarthritis (OA), with less side effects.

An example of this invention is the product, GENOFLEX JOINT HEALTH (GFJH). Salugen's first product will be GENOFLEX Joint Health that offers the person suffering from the signs and symptoms of arthritis, such as pain, inflammation, and joint damage with a custom blend of nutritional ingredients. GFJH address a $1.5 Billion market where there is an unmet clinical need for safe and effective alternative therapies that provide relief from pain, inflammation, joint damage, and the signs and symptoms of arthritis. Over 20 million Americans suffer from Osteoarthritis. Ten million Americans between the ages of 40 and 60 years of age suffer from OA, yet live an active lifestyle remaining in the workforce, raising children, caring for grandchildren, and seeking recreational activities. This condition affecting ten million Americans, part of the baby boom, has been called “Boomeritis”

Arthritis sufferers are more likely to take herbal remedies and nutritional supplements, with 44% of Americans suffering from arthritis taking these forms of alternative therapy.

Many of these patients were taking COX-2 inhibitors for their inflammation because the marketing messages and data suggested that they were safer. With $5 Billion worth of Merck's VIOXX and Pfizer's BEXTRA pulled from the market, and new warnings added to the remaining anti-inflammatories on the market about side effects, such as cardiovascular complications, a huge void has been left with these patients. Thus a market of 4.4 million baby-boomers with “Boomeritis” is waiting for an all-natural, safe and effective DNA-targeted product like GENOFLEX Joint Health to address their ongoing pain and inflammation.

GFJH includes a blend of ingredients that have many clinical studies published demonstrating their effectiveness against pain, inflammation, and joint damage. A consumer would have to take 20 pills a day to receive the same nutritional supplementation. These ingredients include, but are not limited to: glucosamine, chondroitin, quercetin, Ganoderma lucidum, mangosteen extract, and a patented blend called SYNAPTAMINE (U.S. Pat. No. 6,132,724).

TABLE 3
GENOFLEX JOINT HEALTH Basic Formulation or Index Score of 0

| Genoflex Joint Health | | | |
|---|---|---|---|
| standard formulation | | | |
| Supplement Facts | | | |
| Serving Size: 2 tablets three times a day | | | |
| Servings per container: 90 | | | % Daily |
| Amount per Serving | | | Value |
| Proprietary Blend** - Synaptamine ™ | 833 | mg | * |
| DL-Phenylalanine (DLPA) | 500 | mg | * |
| Chromium Picolinate | 200 | mcg | * |
| Rhodiola Rosea | 33 | mg | * |
| L-Tyrosine | 300 | mg | * |
| Bromelain 2400 GDU/gm | 150 | mg | * |
| Boswellia extract (65% boswellic acid) | 100 | mg | * |
| Chondroitin Sulfate | 900 | mg | * |
| Folic Acid | 800 | mcg | 100% |
| Ganoderma Lucidum | 560 | mg | * |
| Glucosamine Sulfate | 1000 | mg | * |
| Hyaluronic Acid (BiocellTM Chicken | 33 | mg | * |
| Collagen Type II) | | | |
| Manganese (as ascorbate) | 16.25 | mg | 812% |
| Mangosteen Extract (40% gamma-mangostin) | 190 | mg | * |
| Quercetin | 100 | mg | * |
| Vitamin C (as manganese ascorbate) | 75 | mg | 125% |
| Vitamin B6 | 20 | mg | 1000% |

* Daily Value not established.
**Synaptamine Complex - U.S. Pat. No. 6,132,724
*** Genoflex Joint Health - Patent-Pending

Salugen will be measuring various genes associated with the efficacy and/or toxicity of these ingredients. The following chart outlines those ingredients.

TABLE 4
GENOFLEX JOINT HEALTH Ingredients that
can be adjusted due to genetic mutations

| Ingredient | Contributing Genes |
|---|---|
| Ganoderma | Ras-Protein and (HLA-DRB1 *0404 and *0101 or |
| Lucidum | PTPN22 R620W) |
| Gamma-Mangostin | Dopamine Receptor D3 Ser9Gly |
| | (-205-G/A, -7685-G/C) |
| Glucosamine | Glutamine: fructose-6-phosphate aminotransferase |
| Sulfate | (GFPT1 or GFPT 2) variant in exon |
| | 14, I471V or 3′ UTR, or glucosamine |
| | 6-P acetyltransferase |
| Chondroitin Sulfate | Aggrecan proteoglycan allele 27 |
| Folic Acid | MTHFR C677T |
| | (heterozygous/homozygous mutant versus |
| | homozygous normal) |

The exact formulation will be determined based upon results from a DNA test for the GENOFLEX Joint Health product. At first, the GENOFLEX Joint Health DNA test measures two genes that can help predict a patient's risk of cardiovascular side effects and arthritis disease progression.

First, Salugen's laboratory will measure the genetic mutation in methylene tetrahydrofolate reductase (MTHFR), specifically MTHFR C677T. In light of the higher cardiovascular risks associated with taking over-the-counter or prescription anti-inflammatory drugs, this component of the GENOFLEX DNA test can provide additional insights. A mutation of this single nucleotide polymorphism has been clinically found to correlate with:
Elevated homocysteine (Hcy) levels in the body;
Risk for hypertension;
Cardiovascular disease inflammation markers;
Prevention of atherosclerosis with tailor-made nutritional supplementation;
Risk for low bone mineral density and osteoporosis;
Incidence of depression;
Risk for stroke; and
Liver toxicity and side effects when taking the most common disease-modifying rheumatoid arthritis medication.

The second genetic mutation measured is Human Leukocyte Antigen DRB 1 (HLA-DRB1). This genetic mutation has been associated with the most disabling forms of arthritis—rheumatoid arthritis—when joints swell and cartilage is damaged. This genetic mutation has been clinically found to correlate with:
Risk for Rheumatoid Arthritis and severity of disease progression;
Risk for joint damage from Rheumatoid Arthritis that can be seen on x-rays; and
Risk for Rheumatoid Vasculitis.

The combination of these two genetic mutations into an index score will provide consumers with a genetic profile offering insights into their healthcare concerns. Equally as important, this genetic index score will guide the customized nutraceutical formulation developed by Salugen for the individual patient.

TABLE 5
EXAMPLE - GFJH Three Formulations Integrated with Two Genes

| genoflex Joint Health | | | | | |
|---|---|---|---|---|---|
| standard formulation | | | | | |
| Supplement Facts | | | | | |
| Serving size: 2 tablets three times a day | | | | | |
| Serving per container: 90 | | | % Daily | Customized Formulations | |
| Amount per Serving | Index 0 | | Value | Index 1 | Index 2 |
| Proprietary Blend** - SynaptamineTM | 833 | mg | * | | |
| DL-Phenylalanine (DLPA) | 500 | mg | * | | |
| Chromium Picolinate | 200 | mcg | * | | |
| Rhodiola Rosea | 33 | mg | * | | |
| L-Tyrosine | 300 | mg | * | | |
| Bromelian 2400 GDU/gm | 150 | mg | * | | |
| Boswellia extract (65% boswellic acid) | 100 | mg | * | | |
| Chrondroitin Sulfate | 900 | mg | * | 1200 mg (+300) | 1200 mg (+300) |
| Folic Acid | 800 | mcg | 100% | 5000 mcg (+4200) | 5000 mcg (+4200) |
| Ganoderma Lucidum | 560 | mg | * | 840 mg (+280) | 320 mg (−240) |
| Glucosamine Sulfate | 1000 | mg | * | 1500 mg (+500) | 1500 mg (+500) |
| Homeopathic Blend | | | * | | |
| Aceonite 12X, Belladonna 12X, Bryonia 12X, | | | * | | |
| Chemonilla 6X, Ferrum Phos 12X, Gelsemium | | | | | |
| 12X, and Berberis 6X) | | | | | |
| Hyaluronic Acid (BiocellTM Chicken Collagen Type | 33 | mg | * | | |
| II) | | | | | |
| Manganese (as ascorbate) | 16.25 | mg | 812% | | |
| Mangosteen Extract (40% gamma-mangostin) | 190 | mg | * | | 100 mg (−90) |
| Quercetin | 100 | mg | * | | |
| Vitamin C (as manganese ascorbate) | 75 | mg | 125% | | |
| Vitamin B6 | 20 | mg | 1000% | | |
| | | | No | Mutation in either AP | Additional or stand alone mutation in |
| | | | Mutations | 27, MTHFR C677T | HLA-DRB1 |

* Daily Value not established.
**Synaptamine Complex - U.S. Pat. No. 6,132,724
*** genoflex Joint Health - Patent-Pending

TABLE 6
EXAMPLE - GFJH Four Formulations Integrated with Genes

| genoflex Joint Health | | | | | | |
|---|---|---|---|---|---|---|
| standard formulation | | | | | | |
| Supplement Facts | | | | Customized Formulations | | |
| Serving Size: 2 tablets three times a day | | | | | Immune | Immune System |
| Servings per container: 90 | | | % Daily | Support | Deficiency | Support |
| Amount per Serving | | | Value | Index 1 | Index 2 | Index 3 |
| Proprietary Blend** - SynaptamineTM | ?? | mg | * | | | |
| DL-Phenylalanine (DLPA) | 500 | mg | * | | | |
| Chromium Picolinate | 200 | mcg | * | | | |
| Rhodiola Rosea | 33 | mg | * | | | |
| L-Tyrosine | 300 | mg | * | | | |
| Bromelian 2400 GDU/gm | 150 | mg | * | | | |
| Boswellia extract (65% boswellic acid) | 100 | mg | * | | | |
| Chrondroitin Sulfate | 900 | mg | * | 1200 mg | | |
| Folic Acid | 800 | mcg | 100% | | | 5000 mcg |
| Ganoderma Lucidum | 560 | mg | * | | 320 mg | 840 mg |
| Glucosamine HCL | 1000 | mg | * | 1500 mg | | |
| Homeopathic Blend | | | * | | | |
| Aceonite 12X, Belladonna 12X, Bryonia 12X, | | | | | | |
| Chamonilla 6X, Ferrum Phos 12X, | | | | | | |
| Gelsemlum 12X, and Berberis 6X) | | | * | | | |
| Hyaluronic Acid (BlocellTM Chicken Collagen Type I: | 33 | mg | * | | | |
| Manganese (as ascorbate) | 16.25 | mg | 812% | | | |
| Mangosteen Extract (40% gamma-mangostin) | 190 | mg | * | | 100 mg | |
| Quercetin | 100 | mg | * | | | |
| Vitamin C (as manganese ascorbate) | 75 | mg | 125% | | | |
| Vitamin B6 | 20 | mg | 1000% | | | |
| | | | | AP 27 or | HLA-DRB1/ | MTHFR C677T or |
| | | | | GFPT 1/2 | PTPN22 | Ras- |
| | | | | | R620W or DRD3 | Protein |

*Daily Value not established.
**Synaptamine Complex - U.S. Pat. No. 6,132,724
***genoflex Joint Health - Patent-Pending

[image]

[image]

[image]

[image]

As previously mentioned, Salugen plans on developing and commercializing DNA tests and customized nutritional supplements utilizing its business model and methods to deliver individualized nutritional solutions to persons dealing with various healthcare concerns. For this application, we would like to review three other applications: stress & anxiety, body recomposition and diabetes.

ANTI-ANXIETY—Corticotropin-releasing factor (CRF) plays an important role in the mediation of the central and peripheral responses to stress. Alterations in CRF system activity have been linked to a number of psychiatric disorders, including anxiety and depression. In line with a role of brain CRF in the mediation of endocrine, autonomic and behavioral responses to stress, transgenic mice over expressing CRF have been reported to show increased anxiety-related behavior, cognitive impairments and an increased HPA axis activity in response to stress, at least part of which can be attenuated by central administration of a CRF antagonist. Sustained exposure of an individual to stress or high levels of CRF decrease CRF binding sites, desensitize CRF-stimulated cyclic AMP accumulation, and decrease ACTH release by corticotrophs. Central administration of CRF can induce a number of adaptive changes in the brain, including changes in CRF receptor expression in various brain areas.

GENES & ANXIETY—Comparing the mRNA expression patterns from whole brains of mice lacking a functional CRF—receptors 1 (CRF1) to that of mice that has received 40 mg/kg of the CRFR1 antagonist R121919 orally for 0, 1, or 7days, alterations in gene expression seen in knockout mice were reported to mimic subchronic (7 day) treatment with the CRFR1 antagonist. Moreover, microarray analysis of 7256 genes reveled altered gene expression in about 90 genes that was attenuated the antagonist. Known targets of CRFR1 Receptor signaling that were altered included immediate early genes such as Jun/B, Nurr1, and Nurr77.

Recent work by Peeters and associates that profiled gene expression in several brain areas of transgenic mice over expressing CRF. Several genes showed altered expression levels in over expressed CRF mice when compared to their wild type littermates and were confirmed by quantitative PCR. Changes included the following:
Glucocorticoid—
   11-beta-hydroxysteroid dehydrogenase type1;
   FK506 binding protein 5;
   serum/glucosteroid kinase
   Human tryptophan 2,3 dioxygenase
Mylenation—
   Myelin
   Myelin associated glycoprotein
Cell proliferation & extracellular matrix formation—
   Edg2
   Fgfr2
   Decorin
   Brevican
Neurogenesis—
   Neurotensin (NT) receptors-1
   Neurotensin (NT) receptors-2

OPIOIDS & NEUROTROPIC FACTOR—In the pituitary the elevated levels of endogenous opioids (preproenkephalin A and prodynorphin) in the pituitary of over expressed CRF mice are in line with the notion that these opioids represent a major modulatory system in the adaptation of an organism to chronic stress. In this regard Blum et. al. (1989) showed that the known enkephalinase inhibitor dl-phenylalanine reduces stress in a double-blind placebo controlled study. Besides this fact, a multitude of data supports the attenuating role of endogenous opioids in response to stress as a protective action of the organism. Another important finding was that in these mice there was also elevation in the mRNA levels of a very powerful neurotropic brain factor known as Bdnf. This observation agrees with previous reports wherein acute and repeated immobilization stress show increased Bdnf mRNA levels in the pituitary.

INTRACELLULAR CALCIUM—In the pituitary, in over expressed CRF mice, it has been shown that changes in intracellular calcium signaling/sensing were exemplified by modulation of hippocalcin like 1 (Hpcall) expression. Hpcall belongs to the neuronal calcium sensor family of Ca2+-binding proteins that play a role in diverse processes, including modulation of neurotransmitter release, control of cyclic nucleotide metabolism, biosynthesis of phosphoinositides and indirect regulation of ion channels.

NEUROGENESIS—The changes of expression observed in genes encoding proteins involved in mylenation, cell proliferation and extracellular matrix formation suggest changes in the dynamics of neurogenesis in over expressed CRF mice. In support if this are the changes in expression observed mainly in the nucleus accumbens (reward site of the brain), involving Edg2, Id2, Gab1 and Fgfr2 genes.

GLUCOCORTICOID SIGNALING—Tissue glucocorticoid concentrations are determined by corticosterone levels and by two intracellular 11-beta-hydroxysteroid dehydrogenases (type 1 & 2) that locally interconvert active glucocorticoids and inert 11-keto forms. 11-beta HSD1, the predominant isoform in the brain, appears to function predominantly as an 11-beta-reductase, regenerating active glucocorticoids from 11-keto forms. 11-Beta-HSD1 deficient mice show alterations in response to stress and are less sensitive to exogenous cortisol suppression of HPA activation, suggesting a diminished glucocorticoid feedback in these animals. Down regulation of 11-beta-HSD1 in the hippocampus of over expressed CRF hints toward an altered glucocorticoid feedback in these animals. This idea is further strengthened by changes observed in the expression of the immunophilin Fkbp5 gene. The exchange of Fkbp5 for Fkbp4 is an important first step in the activation of the glucocorticoid receptors. Moreover, Fkbp5 is a potent inhibitor of glucocorticoid receptor binding. The observed fkbp5 induction suggests attenuation of glucocorticoid receptors by Fkbp5 in response to persistent high levels of circulating glucocorticoids in over expressed CRF animals. A further indication for an altered glucocorticoid signaling is the upregulation of serum/glucocorticoid kinase (Sgk) mRNA in the cerebellum, nucleus accumbens and temporal area, as the transcription of the serine/threonine protein kinase Sgk is induced by glucocorticoids.

Abnormalities in serotonin levels have been implicated in a wide range of psychiatric disorders including stress. Tryptophan 2,3-dioxygenase (TD02) is the rate—limiting enzyme in the catabolism of tryptophan, the precursor of serotonin. The regulatory, intron, and exon regions of the human TDO2 gene have been sequenced and 12 exons have been identified. In the human gene two polymorphisms consisting if G-T and G-A mutations 2 bp apart in intron 6. The 3′ end of intron 5 showed an extensive CCCCT pentanucleotide repeat that was markedly polymorphic. These polymorphisms have already been associated with Tourette's Syndrome and Substance Use disorder.

NEUROTENSIN—Neurotensin (NT), a tridecapeptide, found in numerous areas of the CNS, exerts a variety CNS effects including hypolocomotion, hypothermia, analgesia and reduced food consumption. Three receptors for NT have been identified (Ntsr1,2,3). Glucocorticoids increase NT mRNA expression and release in the brain. High NT levels in turn, down regulate Ntsr1 and 2 receptors. These genes are effected by chronic stress.

One example of a Salugen DNA Test for a Stress/Anxiety product is GENOSTREX. One example of a proposed anti-stress formula is presented in Table 11.

TABLE 11
Salugen Stress and Anxiety Formulation

| | Ingredient |
|---|---|
| Genetic Factor | and Dosage |
| Proenkephalin, prodynorphin, neurotensin (1, 2, 3) | Passion flower - |
| Bdnf, TD02, Sgk, Fkbp5&4, Edg2, Id2, Gab1 Fgfr2 | 100 mg |
| Proenkephalin, prodynorphin, neurotensin (1, 2, 3) | Kava Kava 25 mg |
| Bdnf, TD02, Sgk, Fkbp5&4, Edg2, Id2, Gab1 Fgfr2 | |
| COMT Proenkephalin, prodynorphin, neurotensin | Rhodiola rosea |
| (1, 2, 3) Bdnf, TD02, Sgk, Fkbp5&4, Edg2, Id2, | 200 mg |
| Gab1 Fgfr2 | |
| COMT Proenkephalin, prodynorphin, neurotensin | Rhodendron 100 mg |
| (1, 2, 3) Bdnf, TD02, Sgk, Fkbp5&4, Edg2, Id2, | |
| Gab1, Fgfr2 | |
| COMT DRD1-5 ANKK1 DAT1 DBH TD02 HTT | dl-phenylalanine |
| HTR1A HTR1D HTR2A HTR2C ADRA2A | 2000 mg |
| ADRA2 | |
| NET MAOA GABRA3 GABRB3 CNR1 CNRA4 | |
| NMDAR1 POMC | |
| COMT NET MAOA DRD1-5 ANKK1 DAT1 | l-tyrosine 500 mg |
| DBH POMC Proenkephalin, prodynorphin, | |
| neurotensin (1, 2, 3) Bdnf, | |
| TD02, Sgk, Fkbp5&4, Edg2, Id2, Gab1 Fgfr2 | |
| COMT NET MAOA POMC Proenkephalin, | L-glutamine 100 mg |
| prodynorphin, neurotensin (1, 2, 3) GABRA3 | |
| GABRB3 NMDAR1 | |
| COMT NET MAOA POMC Proenkephalin, | 5- |
| prodynorphin, neurotensin (1, 2, 3) TD02 HTT | Hyroxytryptophane |
| HTR1A HTR1D | 75 mg |
| HTR2A HTR2C | |
| COMT NET MAOA POMC Proenkephalin, | Chromium |
| prodynorphin, neurotensin (1, 2, 3) TD02 HTT | Picolinate 400 mcg |
| HTR1A HTR1D | |
| HTR2A HTR2C DRD1-5 ANKK1 DAT1 DBH | |
| Hpcall COMT NET MAOA | Pyridoxal phosphate |
| | 20 mg |
| COMT Proenkephalin, prodynorphin, neurotensin | Vitamin B complex |
| (1, 2, 3) Bdnf, TD02, Sgk, Fkbp5&4, Edg2, Id2, | |
| Gab1 Fgfr2 | |
| Hpcall DRD1-5 ANKK1 DAT1 DBH | Calcium citrate |
| | 250 mg |
| Hpcall | Magnesium |
| | ascorbate 150 mg |
| COMT DRD1-5 ANKK1 DAT1 DBH TD02 HTT | Hydroxycitric acid |
| HTR1A HTR1D HTR2A HTR2C ADRA2A | 500 mg |
| ADRA2 | |
| NET MAOA GABRA3 GABRB3 CNR1 CNRA4 | |
| NMDAR1 POMC Proenkephalin, prodynorphin, | |
| neurotensin | |
| (1, 2, 3) Bdnf, TD02, Sgk, Fkbp5&4, Edg2, Id2, | |
| Gab1 Fgfr2 | |
| interferon-_CD8A, or PS1, SREBP-1c, PPAR - | Magnolia 10 mg |
| gamma-2, MGPAT. NYP, AgRP, POMC, CART, | |
| OBR, | |
| Mc3R, Mc4R, UCP-1, GLUT4, C-FOS, C-JUN, | |
| C-MYC, Interleukin 1-alpha, interleukin-1 beta, | |
| interleukin-8, tumor necrosis factor-alpha, | |
| intracellular adhesion molecule, interleukin-10, | |
| genes. | |

The genes involved in this stress gene index include but not limited to the following: Pre-enkephalin A, Prodynorphin, Bdnf, Hpcall, Edg2, Id2, Gab1, Fgfr2, Fkbp5, Flbp4, Serum glucocorticoid/kinase, serine/threonine protein kinase, Ntsr1,2,3 Decorin, Brevican, Myelin, Myelin associated glycoprotein, 11-beta-hydroxysteroid dehydrogenase type1, FK506 binding protein 5, Human Tryptophan 2,3 Dioxygenase (TD02), Jun/B, Nurr1, Nurr77.

TABLE 12
Potential Genes to be included in the Stress Genetic Index

| Gene and Single | Effect | Citation |
|---|---|---|
| Nucleotide | | |
| Polymorphism | | |
| Prodynorphin - PDYN- | Association with schizophrenia | Zhang CS, Tan Z, Lu L, Wu SN, He Y, Gu NF, Feng GY, He L. |
| 946C > G | | Polymorphism of Prodynorphin promoter is associated with |
| | | schizophrenia in Chinese population. Acta Pharmacol Sin. 2004 |
| | | Aug; 25(8): 1022-6. |
| | Increase risk for temporal lobe | Stogmann E, Zimprich A, Baumgartner C, Aull-Watschinger S, Hollt V, |
| | epilepsy in patients with family | Zimprich F. A functional polymorphism in the prodynorphin gene |
| | history | promoter is associated with temporal lobe epilepsy. Ann Neurol. |
| | | 2002 Feb; 51(2): 260-3. |
| | *and DRD3 Gly allele (of | Ventriglia M, Bocchio Chiavetto L, Bonvicini C, Tura GB, Bignotti S, |
| | Ser9Gly polymorphism) | Racagni G, Gennarelli M. Allelic variation in the human |
| | contribute to susceptibility of | prodynorphin gene promoter and schizophrenia. |
| | schizophrenia | Neuropsychobiology. 2002; 46(1): 17-21. |
| Brain-Derived | Associated with anxiety | Jiang X, Xu K, Hoberman J, Tian F, Marko AJ, Waheed JF, Harris CR, |
| Neurotrophic Factor | | Marini AM, Enoch MA, Lipsky RH. BDNF Variation and Mood |
| (BDNF) Val66Met and - | | Disorders: A Novel Functional Promoter Polymorphism and |
| 281 C > A, T allele of the | | Val66Met are Associated with Anxiety but Have Opposing Effects. |
| C270T | | Neuropsychopharmacology. 2005 Mar 16; [Epub ahead of print] |
| | Associated with anxiety | Lang UE, Hellweg R, Kalus P, Bajbouj M, Lenzen KP, Sander T, |
| | | Kunz D, Gallinat J. Association of a functional BDNF polymorphism |
| | | and anxiety-related personality traits. Psychopharmacology (Berl). |
| | | 2005 Jun; 180(1): 95-9. Epub 2005 Jan 26. |
| | Late onset Alzheimer's Disease | Olin D, Macmurray J, Comings DE. Risk of late-onset Alzheimer's |
| | | Disease associated with BDNF C270T polymorphism. Neurosci Lett. |
| | | 2005 Jun 24; 381(3): 275-8. |
| Reward Site | | |
| Edg2 | Regulation of myelin formation | Aston C, Jiang L, Sokolov BP. Transcriptional profiling reveals |
| | | evidence for signaling and oligodendroglial abnormalities in the |
| | | temporal cortex from patients with major depressive disorder. Mol |
| | | Psychiatry. 2005 Mar; 10(3): 309-22. |
| | Edg2 activation by | Yoshida A, Ueda H. Neurobiology of the Edg2 lysophosphatidic acid |
| | Lysophosphatidic acid (LPA, 1- | receptor. Jpn J Pharmacol. 2001 Oct; 87(2): 104-9. |
| | acyl-sn-glycerol-3-phosphate), a | |
| | well-known lipid growth factor | |
| | that is found widely in various | |
| | tissues including brain and is | |
| | reported to drive different | |
| | intracellular signaling pathways | |
| | actions related to neurogenesis | |
| | such as cell rounding and | |
| | proliferation, mediates several | |
| | neurobiological actions related | |
| | to neurogenesis, neuronal | |
| | excitability and survival activity | |
| | on developing and postnatal | |
| | neurons | |
| Id2 (Inhibitor of | Protect from cell apoptosis, an | Gleichmann M, Buchheim G, El-Bizri H, Yokota Y, Klockgether T, |
| differentiation 2) | induced gene during serum and | Kugler S, Bahr M, Weller M, Schulz JB. Identification of inhibitor- |
| | potassium deprivation-induced | of-differentiation 2 (Id2) as a modulator of neuronal apoptosis. J |
| | apoptosis of cerebellar granule | Neurochem. 2002 Mar; 80(5): 755-62. |
| | neurons | |
| Gab1 (Grb2-associated | One of the major adapter | Harada S, Esch GL, Holgado-Madruga M, Wong AJ. Grb-2- |
| binder-1) | molecules downstream of | associated binder-1 is involved in insulin-induced egr-1 gene |
| | growth factor receptor signaling | expression through its phosphatidylinositol 3′-kinase binding site. |
| | | DNA Cell Biol. 2001 Apr; 20(4): 223-9. |
| | Grb2-associated binder-1 (Gab1) | Cunnick JM, Dorsey JF, Munoz-Antonia T, Mei L, Wu J. |
| | is a multisite docking protein | Requirement of SHP2 binding to Grb2-associated binder-1 for |
| | containing a pleckstrin | mitogen-activated protein kinase activation in response to |
| | homology (PH) domain, | lysophosphatidic acid and epidermal growth factor. J Biol Chem. |
| | multiple potential tyrosine | 2000 May 5; 275(18): 13842-8. |
| | phosphorylation sites, and | |
| | several proline-rich sequences. | |
| | Gab1 becomes tyrosine- | |
| | phosphorylated in cells | |
| | stimulated with growth factors, | |
| | cytokines, and ligands for G | |
| | protein-coupled receptors. A | |
| | major Gab1-binding protein | |
| | detected in cells treated with | |
| | extracellular stimuli is the | |
| | tyrosine phosphatase, SHP2. | |
| Fibroblast Growth Factor | FGFR2 - SNP located at 2,926 | Ingersoll RG, Paznekas WA, Tran AK, Scott AF, Jiang G, Jabs EW. |
| Receptor 2 (FGFR2) | bp of coding sequence | Fibroblast growth factor receptor 2 (FGFR2): genomic sequence and |
| | | variations. Cytogenet Cell Genet. 2001; 94(3-4): 121-6. |
| | FGFR2 - Ser351Cys mutation | Gripp KW, Stolle CA, McDonald-McGinn DM, Markowitz RI, |
| | | Bartlett SP, Katowitz JA, Muenke M, Zackai EH. Phenotype of the |
| | | fibroblast growth factor receptor 2 Ser351Cys mutation: Pfeiffer |
| | | syndrome type III. Am J Med Genet. 1998 Jul 24; 78(4): 356-60. |
| FKBP5 | Glucocorticoid receptor- | Binder EB, Salyakina D, Lichtner P, Wochnik GM, Ising M, Putz B, |
| | regulating cochaperone of hsp- | Papiol S, Seaman S, Lucae S, Kohli MA, Nickel T, Kunzel HE, |
| | 90, which triggers adaptive | Fuchs B, Majer M, Pfennig A, Kern N, Brunner J, Modell S, Baghai T, |
| | changes in glucocorticoid | Deiml T, Zill P, Bondy B, Rupprecht R, Messer T, Kohnlein O, |
| | receptor and, thereby, | Dabitz H, Bruckl T, Muller N, Pfister H, Lieb R, Mueller JC, |
| | hypothalamic-pituitary-adrenal | Lohmussaar E, Strom TM, Bettecken T, Meitinger T, Uhr M, Rein T, |
| | (HPA)-axis regulation | Holsboer F, Muller-Myhsok B. Polymorphisms in FKBP5 are |
| | | associated with increased recurrence of depressive episodes and rapid |
| | | response to antidepressant treatment. Nat Genet. 2004 |
| | | Dec; 36(12): 1319-25. Epub 2004 Nov 21. |
| myelin oligodendrocyte | Associated with anxiety and | Zai G, Bezchlibnyk YB, Richter MA, Arnold P, Burroughs E, Barr CL, |
| glycoprotein (MOG), a | obsessive compulsive disorder | Kennedy JL. yelin oligodendrocyte glycoprotein (MOG) gene is |
| tetranucleotide TAAA | | associated with obsessive-compulsive disorder. Am J Med Genet B |
| repeat (MOG4), | | Neuropsychiatr Genet. 2004 Aug 15; 129(1): 64-8. |
| C10991T SNP | | |
| Tryptophan 2,3 | rate-limiting enzyme in the | Comings DE, Muhleman D, Dietz G, Sherman M, Forest GL. |
| dioxygenase (TDO2) | catabolism of tryptophan, the | Sequence of human tryptophan 2,3-dioxygenase (TDO2): presence of |
| | precursor of serotonin, | a glucocorticoid response-like element composed of a GTT repeat |
| | associated with TS, autism, | and an intronic CCCCT repeat. Genomics. 1995 Sep 20; 29(2): 390-6. |
| | stress, and substance abuse | |
| | disorders | |
| | | Comings DE, Gade R, Muhleman D, Chiu C, Wu S, To M, Spence M, |
| | | Dietz G, Winn-Deen E, Rosenthal RJ, Lesieur HR, Rugle L, |
| | | Sverd J, Ferry L, Johnson JP, MacMurray JP. Exon and intron |
| | | variants in the human tryptophan 2,3-dioxygenase gene: potential |
| | | association with Tourette's syndrome, substance abuse and other |
| | | disorders. Pharmacogenetics. 1996 Aug; 6(4): 307-18. |
| | | Nabi R, Serajee FJ, Chugani DC, Zhong H, Huq AH. Association of |
| | | tryptophan 2,3 dioxygenase gene polymorphism with autism. Am J |
| | | Med Genet B Neuropsychiatr Genet. 2004 Feb 15; 125(1): 63-8. |
| Serum- and glucose- | Glucocorticoid signaling | Busjahn A, Aydin A, Uhlmann R, Krasko C, Bahring S, Szelestei T, |
| regulated kinase (SGK 1) | | Feng Y, Dahm S, Sharma AM, Luft FC, Lang F. Serum- and |
| SNP Intron 6, Exon 8 | | glucocorticoid-regulated kinase (SGK1) gene and blood pressure. |
| (CC, CT, TT) | | Hypertension. 2002 Sep; 40(3): 256-60. |

BODY RECOMPOSITION—“Weight loss,” “weight gain” and “weight management” are the most common terms used to express changes in body composition, particularly regarding fat mass. However, this patent application will present evidence showing that focusing on “weight” as an accurate measuring criteria poses a contradiction to the natural sequence of processes in recompositional metabolism, creates inappropriate expectations and does not provide a correct and accurate perspective for evaluating healthy changes in body composition, as fat is the lightest of pertinent macro molecules. More importantly, fat is usually the last to go in the body recomposition process; therefore, creating short-term expectations is erroneous. Fat metabolism is influenced by many factors from genetics to lifestyle and the efficiency of energy metabolism. Existing sustainable healthy body composition and improve healthy fat loss. Commercialized “weight loss” programs, even medically supervised versions, do not consider the “bi-phasic” nature of genetically regulated set point “defense response” mechanisms that mandate preservation of body fat stores against famine and survival threats simulated by aggressive weight loss tactics during phase 1. Further, existing tactics place an erroneous emphasis on caloric intake to the exclusion of considering nutrient quality and density of those calories, a factor far more important to metabolic competence than calories alone.

This patent application provides support for a novel body recomposition healthcare concern called neurogenobolics, or the neurologic, genetic, and metabolomic factors contributing to body composition. This present invention provides support for this related healthcare condition, NeuroGenobolic Deficiency Syndrome (NGDS) which accounts for the deficiencies in metabolic competence brought on by 1) the “excessive compensatory” expression of genetic survival mechanisms provoked by chronic and significant dietary stasis and concomitant nutrient deficiencies, 2) exacerbated by yo-yo BMI induced unhealthy deprivation/stimulation tactics, 3) processed through genetic predispositions involving the brain's reward management system, energy management system, stress and inflammation management system, immune system, and 4) the interplay of each system as they're manifested through the endocrine and metabolomic system. The present invention also defines a healthy body mass management technology involving Salugen's business model to customize nutraceutical formulations based upon a patients genes.

This invention provides for a custom formula that can be customized based upon an individual's genetic make-up, and method to safely and naturally induce effective body recomposition and achieve healthy body mass management objectives is presented. This novel technology contrasts with existing tactics to manipulate body composition in that it is based on the fact that sufficient nutrition (as opposed to just calories) is required to fund a wide range of factors involved in achieving healthy and efficient metabolic function. This technology combines synergistic nutraceutical ingredients necessary to simultaneously address symbiotic mechanisms that promote healthy metabolism in the energy management system, stress and inflammation management system, the pleasure/food craving management system (controlled by the brain), the immune management system and the neuroendocrine system. Importantly, these five systems are homeostatic and intimately interactive and interdependent in ensuring optimal metabolic function. This novel nutraceutical technology optimizes genetically programmed energy expenditure and storage functions, without inducing “Yo Yo” rebound weight gain consequences. In contrast to conventional short term expectations, “weight loss” might not be expected since the need to improve the health of the cellular energy producing apparatus should first result in increased muscle density and resultant weight “gain” needed to promote healthy, efficient and permissible fat oxidation and loss. In fact, a more normal and expectable (and healthy) sequence of events might include initial water weight loss, increased muscle density and weight (muscle is heavier than fat and water) followed by permissible fat loss, which could take many months to achieve, depending on gender, race and various other genetically regulated survival responses. (In general, women experience greater difficulty with permissible fat loss due to having a greater quantity of genetic protective mechanisms than men. Such a sequence could and has contributed to disappointment with short term “weight loss” results and abandonment of more intelligent programs that would lead to sustainable fat loss in the healthy body recomposition dynamic.

Various minerals have been shown to be important in funding events leading up to and promoting healthy carbohydrate metabolism, insulin function, energy production, fat oxidation, serotonin release and availability in the brain, blood lipid metabolism and improving the success of fat loss and body composition management efforts. Potassium and calcium salts of (−)HCA have been shown to effectively blunt the conversion of excess carbohydrate into fat, promote fat oxidation, enhance serotonin release and availability in the brain, promote healthy blood lipid levels and improve the success of weight management efforts. Passion Flower has demonstrated stress reduction effects that lead to the lowering of cortisol, reducing the accumulation of excess abdominal fat. A novel oxygen-coordinated chromium nicotinate has been shown to improve insulin sensitivity, promote lean muscle-sparing benefits (enhancing energy metabolism) and enhance fat loss in overweight subjects. This Patent Application will also provide significant evidence to substantiate the existence of Reward Deficiency Syndrome in Obesity and the role of catecholaminergic pathways in aberrant substance seeking behavior, in particular cravings for carbohydrates. The genetic basis for generalized craving behavior will be established. Evidence to support the augmentation of precursor amino acid therapy and enkephalinase and COMT inhibition leading to enhanced levels of neurotransmitters: serotonin, enkephalins, GABA and dopamine/norepinephrine will also be provided. Utilization of this proposed nutraceutical formula has a generalized anti-craving effect and can inhibit carbohydrate bingeing, inducing significant healthy fat loss and relapse prevention. The premise for combining sufficient levels of these ingredients and ingredient complexes is not obvious, and the outcome not anticipated. This is the first time the components of this formula have been combined, and at the dosage levels indicated, to promote successful and sustainable body recomposition management results.

Based on the premise of this patent application, the novel nutraceutical technology presented herein provides ample evidence that the term “weight loss” is a misnomer. This term “weight loss” (or any terms using the “weight” language reference) appearing in quotations is deliberately misused herein to emphasize the point of how conventional tactics (and language) contribute to erroneous, but unquestionably accepted, dogma. Current “weight loss” tactics, for the most part, are based on inducing calorie intake deprivation and artificially stimulate, deprive or inhibit the body's genetically programmed energy expenditure, storage, regulatory and management processes. These types of tactics include, but are not limited to:
Central Nervous System Stimulates (CNSS) that artificially stimulate the rate of calorie burning (Basal Metabolic Rate [BMR]).
Appetite Suppressants
Fat Blockers
Starch Blockers
Diuretics (Water Pills)
Low Calorie Diets
Low Food Diets
Meal Replacement Programs (Diet Shakes, bars, etc.)
High Protein Diets
High Carbohydrate Diets
Low/No Carbohydrate Diets
Low Fat Diets
Pre-Meal Fiber/Water “Fill-You-Up” Programs
Fruit and Fruit juice “Rapid “weight loss”” Programs
Over Night “weight loss” Programs
Vegetable Soup Diet Programs
Liposuction
Radical Digestive Tract Surgeries
Acupuncture
Laxatives
Hypnosis

Many of these tactics are used individually or in combination to achieve rapid “weight loss” results. As stated, the primary goal of these tactics is “weight loss” and/or image enhancement. These objectives are usually pursued without regard for or knowledge of the impact on health, the body's natural genetically mandated homeostatic response to such tactics, or the fact that depriving the body of resources essential to maintain health is counterproductive. Essentially, these types of tactics simulate the circumstances of a famine and induce genetically programmed energy conservation responses. In addition, at some point in the energy conservation sequela, increased appetite can result. Alarmingly, many of these tactics are approved, administered and/or supervised by medical or health professionals. While initially appearing to promote “weight loss” (phase 1), such tactics are destined to fail as gene-induced recalibration of energy management and storage instructions homeostatically adjusts to the artificially imposed influence of such tactics, generally by lowering the basal metabolic rate, increasing energy storage requirements and promoting increased fat retention (phase 2). Chronic and repeated attempts to lose weight with such tactics are referred to as the yo-yo weight gain rebound effect. This phenomenon is responsible for ever-increasing frustration, anxiety and a sense of helplessness caused by the out-of-control “weight loss”/gain juggernaut.

Ultimately, obesity is an energy-balance and nutrient deficiency-induced famine disorder characterized by a survival gene induced increase in fat storage, lowering of the Basal Metabolic Rate (to conserve energy) and increase in appetite. Following circumstances when a simulated famine is induced, certain genes, programmed to resist loss of body fat, prevail. This programmed genetic predisposition is responsible for down-regulating the resting metabolic rate (RMR) in response to dietary and caloric restriction, which is significantly disrupted following rapid “weight loss” regimens, like those tactics indicated above. Over-consumption of food, especially nutritionally deficient high calorie food (excess energy intake), is a normal consequence contributing to weight gain and obesity. A resistance to the hormone leptin also characterizes common obesity. Insulin has been shown to increase leptin secretion by 25%. Ample evidence demonstrates that insulin resistance is also a primary contributor to obesity, suggesting that insulin resistance induced hyperinsulinemia can provoke leptin resistant hyperleptinemia with a consequential increase in fat synthesis and storage in adipocytes, characteristic sequela of Syndrome X or Metabolic Syndrome. Further, adipocytes from fatter animals secrete more leptin and a correlation between intracellular ATP concentration and the rate of leptin secretion appears to exist as such, leptin concentration correlates positively with percent body fat. A low resting metabolic rate (RMR) for a given body size and composition, a low rate of fat oxidation, and low levels of physical activity are risk factors for weight gain and common traits of obese individuals It has been shown that a decrease in body weight as fat mass and fat free mass is accompanied by a greater decrease in resting energy expenditure (REE) and fat oxidation.

The present invention involves effective fat loss and body recomposition strategies that address the energy management pathways to simultaneously improve insulin, serotonin and fat oxidation metabolism; potentiate a healthy increase in RMR and energy expenditure; and blunt excessive appetite cravings, given proper adequate nutrient and energy intake. The technology of the present invention replenishes the nutritional needs of at least five important systems, which are essential to healthy weight management as follows:
1. The biochemical mechanisms involved in nutrition and energy management regulating intake, expenditure and storage controls and feedback;
2. Attenuation of the effects of chronic stress and inflammation (which overburden the endocrine system and can cause things like excessive cortisol production) reducing fat storage;
3. The pleasure seeking needs and reward circuitry of the brain, influencing psychological and emotional need-induced food cravings;
4. Promotion and support of healthy immune system function (involved in catalyzing survival response to metabolic threats; and
5. Supporting and maintaining optimal health of the neuroendrocrine system through which the majority of metabolic signaling is processed.

Nutritional & gene expression deficiencies in the Reward neurochemical pathway limit the brain's reward resources (specific neurotransmitters) and are responsible for a condition called “Reward Deficiency Syndrome, (RDS)” which causes excessive cravings.

TABLE 13
Summary of Studies of the Human D2 Receptor Gene and the Deficiency
Syndrome Behavior-Obesity

| | Polymorphic | | | | | |
|---|---|---|---|---|---|---|
| Investigation | Loci | Type Study | Population Studied | Parameter | Results | Comment |
| Blum et al (1999)¹ | A1 | Association | Morbidly Obese Males | Percent | Positive | DRD2 A1 accounted for 45.9 percent of |
| | | | and Females | Body Fat | | the variance associated with percent body |
| | | | | | | fat as compared with “super controls”. |
| Blum et al (1999)² | A1 | Association | Morbidly Obese Males | Response | Positive | Change in fat weight, change in body |
| | | | and Females | to Change | | weight, percent change in weight, and |
| | | | administered 40 mcg of | in Body | | body weight change in kgms all were |
| | | | CrP/day | Composition | | significant in A2/A2 group and non |
| | | | | | | significant in the A1/A2 and A1/A1 |
| | | | | | | carriers |
| Blum et al (1996)³ | A1 | Association | Obese Patients with a | Obesity | Positive | Association of DRD2A1 allele and |
| | | | Body Mass Index Over | | | patients with a Body Mass Index over 25. |
| | | | 25. Risk factors include | | | Significant increase of A1 percent |
| | | | co-morbid substance | | | prevalence with increasing severity of |
| | | | abuse disorder | | | substance dependence. |
| Blum et al (1994)⁴ | A1 | Association | Obese and control | Obesity and | Positive | A1 allele was present in 25 percent of |
| | | | probands | electro- | | probands having zero risk factors |
| | | | | physiology | | compared to 66 percent of obese subjects |
| | | | | | | with risk factors. This work confirms the |
| | | | | | | association of p300 abnormalities and the |
| | | | | | | A1 allele in obesity. |
| Comings et al | A1 | Association | Young Morbidly Obese | Obesity | Positive | OB and DRD2 genes were additive in |
| (1996)⁵ | | | females | | | their contribution to overall variance in |
| | | | | | | BMI. These two genes accounted for 22.8 |
| | | | | | | percent of BMI variance. |
| Comings et al | DRD2 Haplotypes | Association | Undifferentiated | Obesity | Positive | Undifferentiated obese patients in terms of |
| (1993)⁶ | Intron 6- | | overweight subjects and | | | macro-selection associated with haplotype |
| | Exon 7 | | controls | | | IV. |
| Noble et al (1994)⁷ | A1 | Association | Characterized | Obesity/ | Positive | Prevalence of A1 allele increases in obese |
| | | | overweight obese | carbohydrate | | patients compared to controls. While there |
| | | | patients and non-obese | binging | | was no association with cardiovascular |
| | | | controls and associated | | | factors, a positive association was found |
| | | | risk factors | | | with parental alcoholism and carbohydrate |
| | | | | | | binging. |

This essential body mass management product consists of an amino-acid enkephalinase inhibitor, a natural, herbal catecholamine-o-methyltransfersae (COMT) inhibitor minerals, vitamins, herbals and trace metals as well as a starch blocker. The product is designed to promote normal physiological drive especially in individuals prone to addictive behavior with emphasis on carbohydrate bingeing. Based on a number of clinical trials, the Salugen body mass management product essential is designed to affect abnormal cravings for glucose either simple or in complex form and enhance resting metabolic rate. This technology is stimulant-free.

In terms of the obesity epidemic no condition has received more treatment or mistreatment than obesity or overeating or eating disorders. There seems to be no end to “weight loss” gimmicks, plans, and books. The shortcoming of most of these plans is their focus is taking off pounds as quickly as possible without consideration of underlying conditions that cause people to increase fat storage easily, lose it with difficulty, and regain it quickly after they have struggled to lose it. Obesity is a major public health problem in the domestic and international population. Recent data estimate that between one-third to one-half of the US population is obese. Extensive research aimed at developing treatments to maintain “weight loss” has shown little success. Obesity is resistant to treatment and 90 to 95 percent of people who “lose weight” subsequently regain it, as much as two-thirds of it within one year and almost all of it within five years. There are a number of significant factors that can effect the body's need to increase fat storage; genetics, environment, diet composition, lifestyle, family society and culture. In this regard, an individual's genetic code can determine basal metabolic rate, neurotransmitter function, regulatory peptide levels, and other variables that may put someone at greater risk of increased, excessive and aberrant fat storage (IAFS). There is another even more important facet to the genetic tendency for IAFS than genes that control fat storage or metabolic rates. This is in the genes that control our desire “to binge or not to binge”. These are the “reward genes”. The understanding of neurochemistry, genetics, metabolic rates and energy expenditure, carbohydrate bingeing, body types, lipid anabolism and catabolism, caloric intakes and Syndrome X will provide the basis for polygenetic diagnosis and treatment of obesity.

This present invention, Salugen's weight management product, is a unique, patented (U.S. Pat. No. 6,132,724, as well as other patents issued and pending including a new terminal disclaimer instant patent to be issued in the US {a notice of allowance has be mailed and issue fee paid]) scientifically advanced product that provides a multi-nutritional approach to normal brain function. It supplies your brain with a propriety blend of amino-acids to mimic the Brain Reward Cascade providing proper balance consisting of chromium salts to enhance penetration of select precursor amino-acids along with 5-hydroxytryptophane to increase natural blood tryptophan to assist in the synthesis of serotonin, minerals, vitamins, riboflavin and folic acid to act as co-factors for the production of neurotransmitters. A key to the anti-craving action of the product is the natural compound D-phenylalanine. This substance, found in frog skin among other natural sources, is a known inhibitor of amino-peptidases. This activity increases opioid peptides in the central nervous system, which indirectly increases the release of neuronal dopamine. Twenty percent of the L-phenylalanine is converted to dopamine. The addition of tyrosine also acts as a precursor to the synthesis of dopamine. The pyridoxal-5-phosphate is co-factor in the synthesis of monoamines.

Optional Ingredients—Other natural substances could be added such as Gymnema sylvestre leaves, from a native tree of Africa and India. This botanical has the remarkable ability to block out certain taste sensations, especially that of sweetness. This herb is extremely popular in Japan and is included in diabetic, and hypoglycemic formulas. Modem research has found the gymnemic acid, the active ingredient, is a natural blocker of the “sweet tooth” by reducing glucose taste and also blocks sugar absorption into the body. A clinical study also suggests that an extract of Gymnema can significantly enhance liver and pancreatic function. In addition, ginger and caffeine can effect energy expenditure and the herb Citrus aurantium provides a synephrine base to promote more active energy metabolism and assist the fat loss process. As part of a healthy lifestyle and diet plan to promote an increase in metabolic rate activated pyruvate in the form of potassium glycerophosphate can also be included. The addition of calcium and magnesium assists in the regulation of neurotransmitter release (see example ingredients).

Fortunately, if a broad menu of amino acids is available in sufficient quantity, the brain appears to have the ability to choose from the menu the one or ones needed to manufacture more of the neurotransmitter that is deficient. Based on the patents and technology afforded to us, the following nutrients are scientifically formulated and have been clinically tested for over 20 years and have relevance to the problem defined as “Reward Deficiency Syndrome”, more specifically-overeating and carbohydrate bingeing. However, the work to date supports a generalized anti-craving claim.
D-Phenylalanine, to inhibit enkephalinase, the enzyme that metabolizes or breakdown enkephalins, thereby increasing the availability of enkephalins and, presumably, making more dopamine available at the reward sites especially under stressful conditions (see above for claim support).
L-Phenylalanine, to stimulate the production of dopamine, and/or increase norepinephrine levels in the reward area of the brain. The major problem with this amino acid is that it could compete with other amino acids such as blood borne l-tryptophan and l-tyrosine at the large neutral amino-acid brain carrier system. However, other data demonstrates for the first time that the synthesis and release responses to some dopaminergic agents may be elicited from synaptosomal dopamine, which is formed by the hydroxylation of phenylalanine. Amphetamine and Cogentin increased the release of dopamine formed from 14C-phenylalanine in rat caudate nucleus synaptosomal preparation and concomitantly stimulated the synthesis. Amfoelic acid also caused a net release of that dopamine. In conclusion, the results suggest that synaptosomal particles represent a unit capable of synthesizing dopamine from l-phenylalanine and that synthesis from this precursor may be under the regulatory control of the particles (see above for support).
L-glutamine, to increase brain GABA levels at receptors associated with anxiety. Its major use is to maintain balance in case of over inhibition by D-phenylalanine.
L-5-hydroxytryptophan (or its natural form)—The effect of systemic administration of 5-hydroxy-l-tryptophan on the release of serotonin in the lateral hypothalamus of the rat in vivo as examined utilizing brain microdialysis. Administration of 5-HTP caused an immediate increase of the 5-HT in dialysates, which was long lasting and dose dependent. When calcium was omitted from the perfusion medium, thereby limiting exocytosis, levels of basal 5-HT were significantly decreased and the 5-HTP-induced response of 5-HT was markedly attenuated (see above for support).
Pyridoxal-5-phosphate, the active ingredient of vitamin B6 to serve as a co-factor in the production of neurotransmitters and to enhance the gastrointestinal absorption of amino acids.
Chromium Salts (Nicotinate and Picolinate), have a number of metabolic effects including: increase of insulin sensitivity; reduction of cholesterol; reduction of percent body fat; reduction of weight loss; maintaining muscle mass promoting lean; enhancing body composition; promotes brain serotonin production (see above) Administration of 600 mcg elemental chromium as NBC (ChromeMate®) in two divided doses daily over a period of 2 months to African-American women with a moderate diet and exercise regimen influenced weight and fat loss and sparing muscle and body composition. Another study at the University of Texas found that young obese women consuming 400 mcg of elemental chromium as NBC per day with exercise experienced significant weight loss over an eight week period. Other studies in Fisher F344/BN rats using a chronic dose of 400 mcg of elemental chromium per day and no adverse effects were observed in body and organ weights, and blood chemistries.
Carnitine (optional ingredient), promotes fat metabolism.
Calcium, promotes neurotransmitter release based on many studies (see above for support).
Rhodiola rosea—Several clinical trials with double-blind placebo controls in Russia provide evidence that R. rosea possess positive mood enhancing and anti-stress properties with no detectable levels of toxicity. Generally, R. rosea extract has been shown to have a positive influence on the higher nervous system, increasing attention span, memory, strength and mobility of the human body, and weight management. It is believed that R. rosea can act as a COMT inhibitor where brain levels of serotonin and dopamine have been observed. Studies by Saratikov and Marina suggest that R. rosea can increase the level of neurotransmitters by 30 percent and decrease COMT activity by 60 percent. In the weight management area there are double-blind studies with regard to weight loss and fat mobilization (see above for support).
Rhododendron—is an Asian plant which has important weight loss benefits. Studies in conjunction with Rhodiola 200 mg in combination with Rhododendron 100 mg in double blind studies show significant human weight loss.
(−)—Hydroxycitric acid (HCA) [optional ingredient] has been reported to cause weight loss in humans without stimulating the central nervous system. HCA is derived from the fruit rinds of Garcinia cambogia, which exhibits a distinctive sour taste and has been used for culinary purposes on southern Asia. It has been reported to reduce food intake in experimental animals, suggesting its role in the treatment of obesity. FCA is a competitive inhibitor of ATP-citrate lyase, an extra-mitochondrial enzyme involved in the initial steps of de novo lipogenesis. Consequently, HCA reduces the transformation of citrate into acetyl coenzyme A, a step necessary for the formation of fatty acids in the liver. In addition, there is increased production of liver glycogen in the presence of HCA, which may activate glucoreceptors leading to a sensation of fullness and reduced appetite. Dosage is very important and suboptimal dose of 1500 mg/day has been reported.
Gymnema Slyvestre (Optional Ingredient) helps to promote weight control by its ability to reduce cravings for sweets and control blood sugar levels. A peptide isolated from Gymnema, gurmarin, has also been shown to block the sweet taste of glucose and sucrose in animals. Gurmarin temporarily binds the sweet and bitter receptors on the tongue, thereby blocking the taste sensation and reducing sweet cravings. It is very important to consider a recent study by Preuss et. al (2004) regarding the efficacy of a novel, natural extract of (−) hydroxycitric acid (CA-SX) and a combination of HCA-SX, niacin-bound chromium and Gymnema sylvestre extract in weight management in human volunteers. In a double blind study, in 30 obese subjects for eight weeks the combination compared to controls resulted in reduction of weight loss, and reduction in BMI. Food intake was also reduced. The daily dosage of the HCA-SX was 4,667 mg., Chromate was 400 mcg of elemental chromium, and Gymnema sylvestre was 400 mg (providing 100 mg gymnemic acid).
Hoodia gordonii extract—This substance has food appetite suppression potential. Its combination with Synaptamine has never bee utilized and is not obvious.
Zinc—Zinc plays multiple roles in proper insulin function. Zinc is needed to help the pancreas produce insulin, to allow insulin to work more effectively, and to protect insulin receptor cells. In healthy individuals, insulin is secreted after carbohydrates are eaten, and this hormone lowers glucose levels in the blood and drives sugar into the cells, where it can be used as fuel for energy. When zinc levels are low, two things can happen. One, the pancreas may not secrete adequate amounts of insulin, so glucose levels remain high. Two, the insulin that is released may not work as effectively as it should. When this happens, glucose cannot enter the cells and remains elevated in the blood.
Vanadyl Sulfate—Vanadyl sulfate is a biologically active form of vanadium, a trace mineral that mimics the action of the hormone insulin. Produced by specialized cells in the pancreas, insulin regulates the metabolism of carbohydrates and protein, breaking down those nutrients into a form that can be utilized by the cells to make energy. If you don't produce enough insulin, or develop a condition called insulin resistance, your body will be unable to maintain normal blood sugar levels. Because of its insulin-like properties, vanadyl is being used by progressive alternative physicians and natural healers to treat diabetes, a condition that is characterized by excess sugar in the blood and urine. Studies show that vanadyl is very effective in normalizing blood sugar levels and controlling conditions such as insulin resistance, or Type II diabetes.
Bitter Melon Extract—Bitter Melon is an herb that has traditionally been used by Ayurvedic (Indian) healers to treat Type II, or adult-onset diabetes. Numerous studies have shown that it can normalize elevated blood sugar levels. Bitter Melon contains an insulin-like polypeptide, polypeptide P or p-insulin (the p is for plant). In one study, glucose tolerance improved in 73 percent of Type II diabetics. In another study, the extract of Bitter Melon produced a 17 percent reduction in glycosylated hemoglobin A1c (an indicator of average blood sugar levels over time) after 7 weeks of use.
Fenugreek Extract—Fenugreek has demonstrated significant anti-diabetic effects in experimental and clinical studies. Two studies in the European Journal of Clinical Nutrition reported that Fenugreek improves glucose tolerance in both Type I and Type II diabetes. Consistent intake of fenugreek stimulates pancreatic function. In patients with relatively mild diabetes, fenugreek significantly reduced both fasting and post meal glucose levels and healthy subjects experienced no change in glucose levels.
Bilberry Extract—Bilberry is widely used as a possible preventive treatment for complications of diabetes. This extract affects many health problems—not the least of which is blood sugar imbalances. Bilberry also improves night vision, strengthens capillaries, reduces blood clotting, and has antioxidant action. Research, done mostly in Italy, has also uncovered bilberry's potential for treating retinal problems from poor blood circulation, diabetes-caused glaucoma, and day blindness.
Cinnamon Extract—According to USDA research, cinnamon helps to control blood sugar levels. Ground Cinnamon—the spice, not the flavoring—helps stimulate the production of glucose-burning enzymes and boosts insulin's effectiveness. In one study, cinnamon made insulin 20 times more capable of breaking down blood sugars. Cinnamon has been used for centuries, with references in ancient Greek and Latin writings.
Jambolan—Jambolan is used for diabetes and diseases of the pancreas. It is a species of cloves used in Ayurvedic medicine. Jambolan is used to treat diabetes because it quickly reduces blood sugar, apparently without side effects. Jambolan may also decrease the risk of a person with diabetes developing atherosclerosis because it contains oleanolic acid, which short-circuits the chemical reactions that make toxic free radicals. Oleanolic acid reduces the action of free radicals in atherosclerosis by 60-90 percent.
Pterocarpus marsupium—Pterocarpus Marsupium has a long history of use in India as a treatment for diabetes. A potent flavonoid in this tree has been shown to help regenerate beta cells in the pancreas as well. Researchers in India studied the effects of this herb on 97 individuals with blood sugar problems and were amazed to find that it helped control blood sugar levels in 69% of them.
Gulvel—Gulvel, also known as Tinospora cordifolia, is a multi-faceted plant. It is widely believed not only to help restore vital energy body, but also to help revive the pancreas. Indeed, it has been found to help balance levels of both blood fats and blood sugar.

HCAMin (optional ingredient)—Mineral salts of Hydroxycitric acid (HCA), a natural plant extract from Garcinia cambogia (GcE), has been reported to safely promote fat loss in laboratory animals and humans without stimulating the central nervous system. Unfortunately, all of the studies examining the effects of HCA on changes in body mass confine the focus of attention to HCA alone and ignore the important role of mineral salts (primarily Ca and K) to which HCA is bonded in the neurometabolic equation. Extensive animal and cell culture studies show that effects of HCA are due to its dose dependent ability to competitively inhibit ATP citrate lyase, the citrate cleavage enzyme. Inhibition of this enzyme decreases the transformation of citrate into acetyl CoA, an essential component for fat biosynthesis. Numerous animal studies used intravenous HCA administration, which circumvents the important influence of digestion on bioavailability, an essential consideration for oral consumption. A review of the literature (unpublished and published) on the effects of HCA in humans reveals inconsistent and inconclusive results. Given that GcEs of HCA are orally ingested in humans, variations in efficacy result from variations in a number of compositional characteristics of the extract that influence bioavailability.

A number of unpublished (as peer-reviewed) studies unanimously conclude that HCA (usually in combination with other natural ingredients presumed beneficial for “weight loss”) facilitates fat loss and promotes effective “weight loss”. Interestingly, with similar durations and dosage levels given approximately 30 minutes before meals, changes in body composition results differ between these studies. The study by Ramos, et al. using GcE alone at 500 mg t.i.d. for eight weeks, resulted in a 4.1 kg loss of weight, 3.15 times greater than placebo. Caloric intake was adjusted to accommodate participant's theoretical ideal body weight (from 1000 kcal to 1500 kcal/day). The treatment group contained 18 participants (20 started with 2 dropouts) (Ramos et. al., 1995)). No extract concentration of HCA was identified, but is presumed to be 50% HCA. In the Conte study, all participants were placed on a 1200 kcal/day diet. The treatment group of 23 (30 started with 7 dropouts) taking 500 mg GcE (also identified as “Garcinia indica”, a different species) with 100 mcg chromium (as nicotinate) t.i.d. for eight weeks, experienced “weight loss” of 5.05 kg, 2.65 times greater than placebo (Conte 1993). Once again, no extract concentration of HCA is noted, but a 50% HCA level is presumed, based on popular commercial representations.

The Thom study indicates a dosage level of 440 mg of HCA (as opposed to GcE) t.i.d. (1320 mg/day) for eight weeks. All participants were placed on a 1200 kcal/day diet and were instructed to exercise 3 times per week. The mean “weight loss” in the HCA group was 6.4 kg, 1.68 times greater than placebo. A study by Kaats, et al. assessed body fat loss from 1500 mg of HCA/day (The protocol indicates intake of 5.3 capsules/day, suggesting a 60% HCA concentration). The GcE was taken in conjunction with 1200 mg L-Carnitine and 600 mcg chromium (from picolinate) and compared to placebo in 186 subjects (200 began the study) for 4 weeks. All participants were supposed to engage in an exercise regimen calibrated to personal needs. Loss of body fat in the treatment group averaged 1.29 kg compared to 0.64 kg in the placebo (1.88 times greater than placebo. The Girola study investigated the effects of chitosan, combined with a low amount of GcE, making a comparative evaluation impossible. In fact, variables in the studies noted, and others not mentioned, make conclusions about the efficacy of GcE for “weight loss” extremely difficult.

A review of the published literature bolsters skepticism regarding the beneficial effects of GcE and HCA for weight management. The first published study on HCA found GcE failed to produce “weight loss” and fat mass loss beyond that of a placebo. Once again the focus of attention is on the HCA, to the exclusion of other potentially determining factors or characteristics of the extract, such as mineral components. This paper resulted in publication of Letters To the Editor criticizing study methods and raising counterpoints to various aspects of the study that could influence results. Criticisms noted include: low caloric intake of subjects negated influence of HCA at inhibiting lipogenesis, low carbohydrate intake provides insufficient citrate to exceed energy requirements and provoke fat synthesis (reported to occur in a higher calorie or an unrestricted diet), failure to assess HCA effects on satiety or adherence to a low energy diet, dosage of HCA was low compared to levels suggested by previous animal studies, and high fiber intake could reduce absorption of HCA.

A factor that should have been more significant in the assessment of the JAMA study results regards gender difference in lipogenic potential. This factor would be more appropriate as a “dependent variable” rather than an independent variable. There were 5 men in the treatment group and 14 men in the placebo group. It would be expected that an obese male on 1200 kcal/day would lose more fat and relative weight than an obese female on the same diet, which should have a greater influence on interpretation of study results. A closer inspection of the effects of gender distribution on weight loss between treatment and placebo groups may have been more informative.

Additional criticisms of all the GcE studies to date include: failure to characterize the composition of GcE (only HCA level has been reported); failure to determine and confirm the concentrations of form or forms of calcium (in that excessive levels of calcium [used to stabilize HCA] could reduce absorption), solubility, and levels of naturally occurring pectins (high levels could reduce absorption); and no bioavailability assays were performed to determine the dose dependent bioavailability and relative effectiveness of HCA, along with the important mineral salts, for weight loss.

In a double blind, placebo-controlled, randomized crossover study, Kovacs, et. al. investigated the effects of HCA, a combination of HCA and Medium Chain Triglycerides (HCA+MCT) or placebo on satiety and energy intake in normal to moderately obese subjects for 2 weeks. The subjects consumed self-selected diets. All subjects lost weight but reductions did not differ between treatments. The researchers concluded that HCA and HCA+MCT did not increase satiety or effect energy intake compared to placebo in subjects losing weight. Mattes and Bormann evaluated the effects of HCA on satiety in 89 mildly overweight females during a 12-week double-blind, placebo-controlled parallel group study. Subjects consumed 2.4 g of GcE caplets daily (800 mg t.i.d.). While the researchers report the treatment group lost 3.7±3.1 kg versus 2.4±2.9 kg, about 1.5 times greater than placebo, they conclude that HCA had no observable effects on satiety.

In a double blind, placebo-controlled, randomized crossover study, Kriketos et al, examined the effects of 3.0 g (−) HCA/day for 3 days, versus placebo, on sedentary adult males with and without moderate to intense exercise. The researchers monitored energy expenditure (EE), respiratory quotient (RQ) and various metabolic parameters. They concluded that (−) HCA did not alter the short-term rate of fat oxidation in the fasting state during rest or moderate exercise with doses likely to be achieved in humans, while maintaining a typical Western diet.

Differences in the composition and potency of GcEs can affect pH, solubility, bioavailability, and efficacy; and would account for profound differences in results. Varying the number and kinds of co-ingredients is also a confounding factor. Further, omitting details about product specifications, product analysis, other product components and characteristics (aside from HCA) make it virtually impossible to accurately evaluate, compare and explain results from one study to the next. Without such information, given a valid study method, the level of effectiveness of Garcinia cambogia extract, by default, has been attributed solely to HCA, when in fact other components by their presence or absence can significantly contribute to or detract from the therapeutic effect.

In order to be effective, orally ingested HCA must be absorbed to gain access to the plasma and intima of cells. Studies to determine the effectiveness of HCA as a weight management agent are moot if it is not bioavailable and evidence of bioavailability has not been demonstrated. Omission of accurate bioavailability analysis leaves HCA alone responsible for lack of beneficial weight loss evidence. This omission relinquishes the opportunity to explain the significant differences between studies. Schwarz, et al. developed a Gas Chromatography/Mass Spectrometry Method (GC/MS) to accurately identify and quantify plasma HCA levels in humans. Plasma HCA concentrations were measured over 3.5 hour period in fasting and fed subjects ingesting 2 g of HCA. While HCA levels varied between subjects, peak plasma levels were reached in approximately 2 hours. The source of HCA used in this study was a novel stabilized Ca/K 60% HCA GcE (CitriMax HCA-600-SXG-P capsules ˜500 mg, supplied by InterHealth Nutraceuticals). This source is currently patent pending and is the same source used in the formula that is the subject of the present invention and patent application.

The Ca/K moiety of HCA and its effects on the extract's physical and chemical properties appear crucial. HCA's polar functional groups render it susceptible to poor absorption due to the possibility of interactions with other compounds in foods. Bonding of HCA with other salts, individually or in combination could significantly alter physical and chemical properties and the therapeutic effects accordingly. Further, bioavailability and efficacy of HCA (and calcium and potassium) from this material has been demonstrated by its ability to influence leptin metabolism and various metabolic parameters crucial for healthy weight loss. In a randomized, double-blind and placebo-controlled study, Preuss, et al investigated the effects of a novel Ca/K 60% HCA, alone (Group A) and in combination with a patented niacin bound chromium complex (4 mg/d providing 400 mcg chromium) and Gymnema sylvestre (400 mg/d) (Group B). In both groups, (−) HCA intake was 2800 mg/day, given to moderately obese humans consuming a 2000 kcal diet/day and engaging in a walking exercise regimen. Among other factors, body weight, BMI, lipid profile, and serum leptin levels (a marker of obesity gene) were assessed at 4 and 8 weeks. At the end of 8 weeks, supplementation of HCA-SX in Group A decreased serum leptin levels by 36.6%. Body weight and BMI were decreased by 6.3% respectively and food intake decreased by 4%. Total cholesterol LDL and triglycerides were decreased 6.3%, 12.3%, and 8.6% respectively with no significant adverse effects observed. HDL and serotonin levels increased by 10.7% and 40%, respectively. In Group B, serum leptin levels decreased 40.5%, body weight and BMI reduced by 7.8% and 7.9% respectively. Total cholesterol, LDL and triglycerides were reduced by 9.1%, 17.9% and 18.1% respectively, while HDL and serotonin levels increased by 20.7% and 50% respectively. Food intake was reduced by 14.1%. This research demonstrates bioavailability and beneficial effects on various metabolic parameters important for safe healthy weight management for a novel preparation of Ca/K 60% HCA (commercially available as Super CitriMax).

Potassium (K) and calcium (Ca) are important ions for a number of metabolic pathways influencing energy expenditure, leptin metabolism and weight control. Intracellular energy production is important for acute leptin secretion. Potassium and calcium flux may play important roles in coupling intracellular energy production to leptin secretion. Restriction of sodium intake is a common dietary recommendation in the treatment of Syndrome X disorders. However, meta-analyses indicate that bolstering calcium and potassium intake should be the focus of dietary recommendations, rather than restriction of sodium in the management of such disorders as hypertension. Evidence demonstrates that diets rich in Ca, K, and Mg produce a potent antihypertensive effect, reported that rat hearts perfused with glucose, insulin and potassium (GIK) had significantly higher ATP, creatine phosphate, energy chare, and NADP(+) and lower AMP and inosine levels compared with controls after 30 minutes of reperfusion. Reperfusion with GIK improved post-ischemic recovery of contractile function and the myocardial bioenergetic state.

Most neurons use glucose for energy but glucose sensing neurons in the brain use glucose for signaling to regulate neuronal firing and transmitter release. Glucose responsive neurons (GRN) increase their firing rate as brain glucose levels rise, functioning much like pancreatic beta-cells in which glycolysis regulates the activity of the ATP-sensitive K(+) channel. The output of these neurons is crucial to the effector systems, which regulate energy homeostasis. As such, inadequate available K could be a rate-limiting factor in GRN firing, possibly increasing compensatory demands for additional glucose substrate as well. Further, Deriaz et al. showed that chronic changes in serum K concentrations were significantly correlated with changes in energy expenditure. Spanswick et al. reported that leptin hyperpolarizes glucose-receptive hypothalamic neurons in lean Sprague-Dawley and Zucker rats, but not in obese Zucker rats. This hyperpolarization results from activation of a potassium current that is blunted in obese Zucker rats. Other research by Spanswick, et al. suggests that hypothalamic K (ATP) channel function is crucial to physiological regulation of food intake and body weight (36). K is required for cellular energy production via the K (ATP) channel. A low K intake has been shown to alter serum K levels, K homeostasis and suppress cellular energy expenditure. Further, K (ATP) channels reside in the plasma membrane of many excitable cells such as pancreatic beta cells, heart, skeletal muscle and brain, where they link cellular metabolic energy to membrane electrical activity. Insulin action depends on the energy level of target cells and K (ATP) channels are believed to influence glucose transport due to their role in energy homeostasis in the insulin target tissues. Addition of K and Mg phosphates to orange juice significantly increased energy expenditure in obese postmenopausal women, while no effect was seen in lean women or in women consuming the unsupplemented juice. The researchers conclude that addition of K/Mg phosphate to glucose increases postprandial thermogenesis in obese postmenopausal women, but not in lean ones. Nazar, et al. showed that supplementation of calcium, potassium and sodium phosphates increased RMR in 2 groups of overweight women (double blind, placebo controlled, cross-over) on a low energy diet by 12% and 19%. The study found that mineral supplementation improved thyroid plasma T3 levels and T4 to T3 ratio. They concluded that mineral supplementation in obese patients on a low-energy diet enhances RMR irrespective of the rate of “weight loss” and seems to be due, in part, to the influence of the minerals on peripheral metabolism of thyroid hormones.

Dietary calcium also plays a crucial role in the regulating energy metabolism. High-calcium diets have been shown to inhibit fat synthesis and storage in adipocytes and reduce fat storage during over-consumption of an energy-dense diet. High calcium intake (from dairy) was also shown to increase lipolysis and preserve thermogenesis during caloric restriction, accelerating “weight loss”. In contrast, low calcium diets have been shown to impede body fat loss. Data gathered from five clinical studies were evaluated to determine the relationship between calcium intake and body weight. The calcium treated subjects in the controlled trial exhibited a significant weight loss compared to the placebo group, across approximately 4 years of observation. A paper by Heaney, et al. states that each 300 mg increment of regular calcium intake is associated with about 1 kg less body fat in children and 2.5-3.0 kg lower body weight in adults. Heaney et al estimates that while calcium intake explains only a fraction of the variability in weight gains, increased calcium intake could reduce the prevalence of overweight and obesity by as much as 60-80%. A related review by Teegarden reports that calcium may play a key role in reducing the incidence of obesity and prevalence of insulin resistance syndrome. The most obvious reason for adequate calcium intake during a body recomposition regimen is that bone turnover is increased during such changes in postmenopausal women, increasing the risk of bone demineralization disorders, like osteoporosis. Calcium supplementation can suppress development of these maladies.

This evidence supports the present invention's claim that adequate intake of dietary K and Ca enhances energy production, leptin and insulin metabolism, enhances dopamine release, and satisfies particular nutrient needs of important pathways required for healthy sustained fat loss, body composition and healthy weight management. It is for this reason that stabilizing (−)HCA with K and Ca ions amplifies the effectiveness of HCA in achieving significant fat loss, improved BMI, upregulation of RMR, and improves energy expenditure and fat oxidation.

“Weight loss” studies using GcE routinely attribute results only to the effects of HCA, evidently believing other components are inert or inactive, and other characteristic properties irrelevant. Many manufacturers, marketers and/or researchers either capitalize on these omissions for monetary gain or are just ignorant about the roles of other important components. Many variable factors need to be known and considered in explaining why effectiveness of HCA materials varies, with many being ineffective. In addition to 60% HCA, a novel IH464 GcE supplies approximately 720 mg of potassium and 495 mg of calcium per 4500 mg daily intake. Considering the questionable effectiveness of HCA in other preparations compared to the Ca/K salt in the novel IH464 GcE, and considering the evidence regarding the benefits of Ca and K at lowering excess body fat and promoting healthy body composition, it is reasonable to conclude that Ca and K may be more important to the needs of body recomposition and fat reduction than HCA. It is clear that Ca and K are important ingredients as used in the formula of the present invention. The calcium and potassium ions in this novel preparation contribute an important role in achieving significant the loss of excess fat by multiple synergistic pathways.

The dried fruit of Garcinia cambogia, also known as Malabar tamarind, is a unique source of (−) hydroxycitric acid (HCA), which exhibits a distinct sour taste and has been safely used for centuries in southeastern Asia to make meals more filling. Recently, it has been demonstrated that HCA-SX or Super Citrimax, is safe when taken orally and that HCA-SX and that HCA-SX us bioavailable in the human plasma as studied by GC-MS. Although HCA-SX has been observed to be conditionally effective in weight management in experimental animals as well as humans, its mechanism of action remains to be understood. In a study by Roy et al in rats, they observed that at doses relevant for human consumption dietary HCA-SX significantly contained body weight growth. This response was associated with lowered abdominal fat leptin expression while plasma leptin levels remained unaffected. Repeated high-density microarray analysis of 9960 genes and certain genes present in fat tissue identified a small set (approximately 1% of all genes screened) of specific genes sensitive to HCA-SX. Other genes, including vital genes transcribing for mitochondrial/nuclear proteins and which are necessary for fundamental support of the tissue, were not affected by HCA-SX. Functional characterization of HCA-SX sensitive genes revealed that pregulation of genes encoding serotonin receptors represent a distinct effect of dietary HCA-SX supplementation.

Passiflora incarnata—Passionflower is a name that as been given to several members of the genus Passiflora. There are more than 40 species in the genus whose origins are in both the tropical and subtropical regions of the western hemisphere. Passionflower was first brought to Europe from Mexico in the sixteenth century by Spanish conquerors. Its main medicinal purpose was that of a calming tea. It is now part of the medicinal herbarium in many countries throughout the world. Passion flowers long history in herbal medicine includes its use as a treatment for colic, diarrhea, dysentery, menstrual pain, skin eruptions, conjunctivitis, hemorrhoids, and muscle spasms. However, the inclusion in this present invention's weight management product involves its central nervous system effects.

One of the problems with this subtropical plant is its identity. While there are a number of alkaloids which have been sold under the rubric of Passionflower, the most important and consistently effective candidate is Passiflora incarnata. The ethnobotanical database on the U.S. Agricultural Research Service's Web site lists the total alkaloid content P. incarnata as 100 to 900 ppm and the total flavonoid content as 1.2-3.9 percent. Twenty-six components fall into two categories: 20 flavonoids (including a cyanogenic glycoside and gynocardine) and 6 alkaloids. Some researchers have ascribed the sedative effects of p. incarnata to indole alkaloids such as Harmane and its relatives' harmaline and harmol. However, others have suggested that P. incarnata's alkaloid content is too small to cause this and other CNS effects and that flavonoids—such as apigenin, luteolin, or their glycosides are more likely to account for CNS bioactivity.

Most recently, scientists have isolated a highly anxiolytic, trisubstituted benzoflavone moiety from a P. incarnata extract. Reports from the literature reveal that this extract has the ability to restore libido on aging male rats, and those who are addicted to tetrahydrocannibinol, to restore fertility and libido that has been reduced by alcohol or nicotine use, and to reduce the anxiety arising from alcohol withdrawal. There are also double-blind randomized studies which suggest that Passiflora extract is as effective substance for the management of generalized anxiety disorder comparative to the drug Oxazepam. There is even evidence that Passiflora in a double-blind randomized controlled trial may be an effect adjuvant in the management of opiate withdrawal of opiates. In addition Passoflora reduced benzodiazepine dependence in mice. In fact, many pharmacological investigations confirm the sedative effects of Passoflora, especially in the P. incarnata form (Krenn, 2002). The present invention of a weight management product was formulated with the knowledge afforded EuroMed (source of the fragmented or cut, dried aerial parts of P. incarnata). According to Dhawan et al. the separated leaves afford the best possible CNS results, and in fact, the selected of the entire aerial parts excluding the flowers may prove to be the optimum approach for pocking up the bioactive plant parts of P. incarnata. The importance of standardization of preparations of Passoflora has been actively studied by Dhawan, especially as it relates to the anxiolytic activity.

In the early 70's, Blum showed the importance of the brain neurotransmitter serotonin as a biological substrate of stress. In fact, induction of stress in rodents was attenuated by injections of the serotonin chemical synthesis depletor Para-Chlora-Phenylalanine (PCA). Others have also shown the involvement of serotonin and dopamine in stress production in both animals and humans. Moreover, work by Blum also showed that amino-acid and enkephalinase inhibition therapy reduced stress in polysubstance abusers as measured in a double-blind-placebo randomized controlled trial in humans using skin conductance levels. These studies seem to dove tail the work reported on the anxiolytic effects of Passoflora. In fact it is very interesting that at least one phytoconstituent is indeed an indole similar to the chemical makeup of serotonin.

It is well known that stress induces the preferential release of the circulatory hormone cortisol in humans. It is well know that lipolysis is the major activity that is involved in the burning of fat in adipose tissue. Ottosson et. al. clearly showed that cortisol significantly reduced the basal rate of lipolysis (p <0.01) and the catecholamine lipolysis stimulators isoprenaline and noradrenalin in vitro. Thus, cortisol will increase rather than decrease fat burning. In addition, the pathogenesis of obesity has been intimately linked to the catecholaminergic regulation of lipolysis and the function of the sympathetic nervous system. Norepinephrine and epinephrine activate lipolysis via B1 and B2 and B3—adrenoreceptors and inhibit it via alpha2—adrenoreceptors, and these neurotransmitters are the most important lipolytic substances on vivo. Defects of the catecholamine-induced lipolysis have been observed in a number of obese subjects, and polymorphisms of the B2 and B 3 receptors. By adding both Passoflora and Synaptamine, the present invention proposes a synergistic effect on stress production and enhanced catecholamine synthesis. We further believe that these ingredients coupled together would induce a reduction of plasma cortisol on humans. This will indeed then enhance lipolysis and increase fat burning.

In essence, this novel formulation which can be customized with Salugen's business model and methods of genetic analysis will promote the synthesis of the brain reward neurotransmitters like serotonin and catecholamines and through its effect on the natural opioids will by virtue of inhibiting GABA cause a significant release of dopamine at the nucleus accumbens. This constant release of possibly therapeutic amounts of dopamine (anti-stress substance) occupies dopamine D2 receptors, especially in carriers of the A1 allele (low D2 receptors and high glucose craving), and over time (possibly 6-8 weeks) effects RNA transcription leading to a proliferation of D2 receptors, thereby, reducing craving for carbohydrates. Evidence for anorectic actions of dopaminergic stimulators like Amphetamines I (ephedra) have been found to work via activation of both D1 ands D2 dopamine receptors. In addition, elucidation of the composition, characteristics and properties of stabilized (−) HCA compounds of GcEs is essential to differentiate effective sources from ineffective and substantiate the actual active ingredients in such mineral-based complexes. Recent research demonstrates intake of 4500 mg/d of a novel IH464 GcE containing 720 mg of K and 495 mg of Ca bound by (−) HCA for 8 weeks, while consuming a 2000 Kcal/d diet, produced safe and effective loss of body fat and improved BMI without stimulating the central nervous system. Other ingredients as listed in the example will also provide important benefits such as anti-craving anti-stress, enhancement of serotonin, energy and metabolism induction, appetite suppression, starch blocking, glucose stabilization, fat burning, and general nutrition, as well as neurotransmitter rebalancing. Collateral benefits of lowered food intake and improved serotonin, insulin, lipid and leptin metabolism provide valuable evidence that this compound addresses multiple pathways in achieving sustainable healthy fat loss and improvements in body mass index while averting the consequences of rapid “weight loss” induced by CNS stimulation and/or calorie deprivation.

Various genes have been associated with susceptibility to diabetes and obesity, risk for poor insulin metabolism, or potential outcomes related to nutrition. The present invention involves using Salugen's algorithms, business model, and methods to customize nutrition for optimized neurogenobolics.

In essence, HCA-SX can up regulate 93 genes and down regulate 18 genes. For example, one important gene that HCA-SX down regulated is the leptin gene. Moreover, a few important genes upregulated by HCA-SX include but is not limited: PDGS, ALdB and LNC2. In terms of neurotransmitters and their receptors HCA-SX did not alter the expression of dopamine receptors (D1-D5), Adrenergic Receptors , Histamine Receptors, Miscarinic Acetylcholine receptors. However, HCA-SX did upregulate serotonin receptors (5-HT-2A, 5-HT 2B, 5-HT-4 & 5-HT-7). These serotonin genes will potentially form in part the Salugen Index Score for the product lines involving HCA.

TABLE 14
Potential Neurogenobolic Genes to be included in a DNA test index that will direct
the customization of a body recomposition nutraceutical

| | | | | |
|---|---|---|---|---|
| Gene and Single | | | | |
| Nucleotide | | | | |
| Polymorphism | Effect | Citation | Nutraceutical | Citation |

Susceptibility to Diabetes and Obesity

| | | | | |
|---|---|---|---|---|
| Pro12Ala | Susceptibility to Type | Horiki M, Ikegami H, Fujisawa T, | Coccina Indica | Yeh GY, Eisenberg DM, Kaptchuk TJ, |
| polymorphism of | 2 Diabetes and | Kawabata Y, Ono M, Nishino M, | | Phillips RS. Systematic review |
| PPARgamma | hypertension | Shimamoto K, Ogihara T. | | of herbs and dietary supplements |
| gene | | Association of Pro12Ala | | for glycemic control in diabetes. |
| | | polymorphism of PPARgamma gene | | Diabetes Care. 2003 |
| | | with insulin resistance and related | | Apr; 26(4): 1277-94. |
| | | diseases. Diabetes Res Clin Pract. | | |
| | | 2004 Dec; 66 Suppl 1: S63-7. | | |
| | Risk for obese subjects | Ghoussaini M, Meyre D, Lobbens S, | | |
| | to get Type 2 Diabetes | Charpentier G, Clement K, Charles MA, | | |
| | with insulin resistance | Tauber M, Weill J, Froguel P. | | |
| | and increased fasting | Implication of the Pro12Ala | | |
| | insulin levels | polymorphism of the PPAR-gamma | | |
| | | 2 gene in type 2 diabetes and obesity | | |
| | | in the French population. BMC Med | | |
| | | Genet. 2005 Mar 22; 6(1): 11. | | |
| E23K Kir6.2 | Susceptibility to Type | Riedel MJ, Steckley DC, Light PE. | | |
| polymorphism | 2 Diabetes, obesity and | Current status of the E23K Kir6.2 | | |
| | increased fat storage | polymorphism: implications for | | |
| | | type-2 diabetes. Hum Genet. 2005 | | |
| | | Feb; 116(3): 133-45. Epub 2004 Nov 23. | | |
| | | | 200 mg/100 g | Kumar GP, Sudheesh S, |
| | | | BW QD | Vijayalakshmi NR. Hypoglycaemic |
| | | | | effect of Coccinia indica: |
| | | | | mechanism of action. Planta Med. |
| | | | | 1993 Aug; 59(4): 330-2. |
| steroid sulfatase | STS “G” allele (n = 36) | Riechman SE, Fabian TJ, Kroboth PD, | 17-keto-DHEA | Villareal DT, Holloszy JO. Effect |
| (STS) gene | had greater acute | Ferrell RE. Steroid sulfatase | 50 mg | of DHEA on abdominal fat and |
| variation | changes in DHEA | gene variation and DHEA | | insulin action in elderly women and |
| | [+4.4 (0.7) vs. +2.0 ng/ml | responsiveness to resistance exercise | | men: a randomized controlled trial. |
| | (0.5), S1; +3.2 | in MERET. Physiol Genomics. | | JAMA. 2004 Nov 10; 292(18): 2243-8. |
| | (0.6) vs. +1.0 ng/ml | 2004 May 19; 17(3): 300-6. | | |
| | (0.4), S30; P < 0.01] | | | |
| | and DHEAS: DHEA [−37 | | | |
| | (11) vs. 5 (7), S30, | | | |
| | P < 0.05] than those | | | |
| | subjects with only an | | | |
| | “A” allele (n = 84). | | | |
| Pro12Ala | A corresponding | Karbowska J, Kochan Z. Effect of | | |
| polymorphism of | increase in peroxisome | DHEA on endocrine functions of | | |
| PPARgamma | proliferator-activated | adipose tissue, the involvement of | | |
| gene | receptor gamma | PPARgamma. Biochem Pharmacol. | | |
| | (PPARgamma) mRNA | 2005 May 16; [Epub ahead of print] | | |
| | expression suggests | | | |
| | that PPARgamma may | | | |
| | be involved in the up- | | | |
| | regulation of | | | |
| | adiponectin gene | | | |
| | expression after | | | |
| | DHEA treatment. | | | |

Risk for Obesity and Poor Insulin Metabolism

| | | | | |
|---|---|---|---|---|
| G82G at the | Obesity and insulin | Ukkola O, Rankinen T, Lakka T, | Gymnema | Yeh GY, Eisenberg DM, Kaptchuk TJ, |
| PTPN1 | metabolism | Leon AS, Skinner JS, Wilmore JH, | Sylvestre 25 mg | Phillips RS. Systematic review |
| IVS6 + G82A | | Rao DC, Kesaniemi YA, Bouchard C. | | of herbs and dietary supplements |
| polymorphism | | Protein Tyrosine Phosphatase 1B | | for glycemic control in diabetes. |
| | | Variant Associated with Fat | | Diabetes Care. 2003 |
| | | Distribution and Insulin Metabolism. | | Apr; 26(4): 1277-94. |
| | | Obes Res. 2005 May; 13(5): 829-34. | | |
| Sulfonylurea | Sur1 deficient impacts | Lam TK, Pocai A, Gutierrez-Juarez R, | Hoodia | MacLean DB, Luo LG. Increased |
| receptor 1 | hypothalamic K(ATP) | Obici S, Bryan J, Aguilar-Bryan L, | Gordonii 500 mg | ATP content/production in the |
| | channels or | Schwartz GJ, Rossetti L. | | hypothalamus may be a signal for |
| | pharmacological | Hypothalamic sensing of circulating | | energy-sensing of satiety: studies of |
| | blockade (K(ATP) | fatty acids is required for glucose | | the anorectic mechanism of a plant |
| | blocker) of their | homeostasis. Nat Med. 2005 | | steroidal glycoside. |
| | activation by fatty | Mar; 11(3): 320-7. Epub 2005 Feb 27. | | Brain Res. 2004 Sep 10; 1020(1-2): |
| | acids, if deficient, then | | | 1-11. |
| | more supplement | | | |
| Pro12Ala | Supplement drastically | Liu F, Kim J, Li Y, Liu X, Li J, | Lagerstroemia | Suzuki Y, Unno T, Ushitani M, |
| polymorphism of | reduces mRNA from | Chen X. An extract of Lagerstroemia | speciosa L. | Hayashi K, Kakuda T. Antiobesity |
| PPARgamma | gene | speciosa L. has insulin-like glucose | (Banaba | activity of extracts from |
| gene | | uptake-stimulatory and adipocyte | Extract) 100 mg | Lagerstroemia speciosa L. leaves on |
| | | differentiation-inhibitory activities in | | female KK-Ay mice. J Nutr Sci |
| | | 3T3-L1 cells. J Nutr. 2001 | | Vitaminol (Tokyo). 1999 |
| | | Sep; 131(9): 2242-7. | | Dec; 45(6): 791-5. |

Leptin Levels and Propensity for Weight Gain, Effect of Exercise on Insulin

| | | | | |
|---|---|---|---|---|
| beta(3)-AR | Affects Leptin Levels | Ramis JM, Gonzalez-Sanchez JL, | Conjugated | Wang YW, Jones PJ. Conjugated |
| Trp64Arg | | Proenza AM, Martinez-Larrad MT, | Linoleic Acid | linoleic acid and obesity control: |
| | | Fernandez-Perez C, Palou A, | (CLA) (from | efficacy and mechanisms. Int J |
| | | Serrano-Rios M. The Arg64 allele of | Safflower Seed | Obes Relat Metab Disord. 2004 |
| | | the beta 3-adrenoceptor gene but not | Oil) (500 mg | Aug; 28(8): 941-55. |
| | | the −3826G allele of the uncoupling | total) . . . 380 mg | |
| | | protein 1 gene is associated with | | |
| | | increased leptin levels in the Spanish | | |
| | | population. Metabolism. 2004 | | |
| | | Nov; 53(11): 1411-6. | | |
| Leptin Gene and | Magnitude of effects | Lakka TA, Rankinen T, Weisnagel SJ, | | |
| Leptin Receptor | from exercise on | Chagnon YC, Lakka HM, | | |
| Gene (R109R | insulin levels | Ukkola O, Boule N, Rice T, Leon AS, | | |
| homozygotes, | | Skinner JS, Wilmore JH, Rao DC, | | |
| LEP A19G | | Bergman R, Bouchard C. Leptin | | |
| polymorphism | | and leptin receptor gene | | |
| and the LEPR | | polymorphisms and changes in | | |
| 109R carriers) | | glucose homeostasis in response to | | |
| | | regular exercise in nondiabetic | | |
| | | individuals: the HERITAGE family | | |
| | | study. Diabetes. 2004 | | |
| | | Jun; 53(6): 1603-8. | | |
| DRD2 gene | Pkarmacogenomic | Paper submitted to International J. | Synaptamine | DLPA 2000 mg, 1-Tyrosine 750 mg, |
| TaqA1 allele | response to chromium | Eating Disorders Blum et. al. | Complex | 5-HTP 100 mg, Vitamin B6 20 mg, |
| | picolinate | Paper by Blum et al submitted to | | Chromium Polynicotinate or |
| | DRD2 A1 allele & | Pharmacogenomics. | | Chromium Nicotinate 400 mcg, |
| | Body fat. | | | Rhodiola Rosea 200 mg, |
| | | | | Rhododendron 100 mg) |
| | DRD2 A1 allele | Paper in press by Blum & Comings, | | |
| | associated with RDS | Neurotoxcity Research. To be | | |
| | behaviors in 5 | presented in Venice, Italy at the IPS | | |
| | generations. | conference.. | | |
| N/A | N/A | N/A | Chromium | Preuss HG, Bagchi D, Bagchi M, |
| | | | Polynicotinate | Rao CV, Dey DK, Satyanarayana S. |
| | | | or Chromium | Effects of a natural extract of(−)- |
| | | | Nicotinate | hydroxycitric acid (HCA-SX) and a |
| | | | (niacin-bound | combination of HCA-SX plus |
| | | | chromium) | niacin-bound chromium and |
| | | | | Gymnema sylvestre extract on |
| | | | | weight loss. Diabetes Obes Metab. |
| | | | | 2004 May; 6(3): 171-80. |

Additional genes contributing to a genetic index on Body Recomposition are: DRD1, DRD2, DRD3, DRD4, DRD5, DAT1, HTT, HTR1A, TD02, DBH, ADRA2A, ADRA2C, NET, MAOA, COMT, GABRA3, GABRB3, CNR1, CNRA4, NMDAR1, PENK, AR, CRF, HTR1D_ HTR2A, HTR2C, interferon-_CD8A, or PS1, ANKK1, TD02, SREBP-1c, PPAR-gamma-2, MGPAT. NYP, AgRP, POMC, CART, OBR, Mc3R, Mc4R, UCP-1, GLUT4, C-FOS, C-JUN, C-MYC, Interleukin 1-alpha, interleukin-1 beta, interleukin-8, tumor necrosis factor-alpha, intracellular adhesion molecule, interleukin-10, genes.

In terms of weight loss ingredients one nutrient Chromium Picolinate, one of the most widely used ingredient in the weight management field, pharmacogenomically responds differentially based on the DRD2genotype. Dr. Blum is current in revision on a publication detailing these findings. Here are some details on the abstract:
Thomas J H Chen, Kenneth Blum, * Gilbert Kaats, E. R. Braverman. Arthur Eisenberg, M. Sherman. K. Davis, D E Comings, R. Wood, D. Pullin. CHROMIUM PICOLINATE A PUTATIVE ANTI-OBESITY NUTRIENT INDUCES CHANGES IN BODY COMPOSITION AS A FUNCTION OF THE Taq1 DOPAMINE D2 RECEPTOR POLYMORPHISMS. International J. Of Eating Disorders (in revision).
Objective: There is still controversy regarding the effects of chromium salts (picolinate and nicotinate) on body composition and weight loss in humans. Thus, we decided to test the hypothesis that typing the obese patients by genotyping the DRD2 gene prior to treatment with Chromium Picolinate (CrP) would result in a differential treatment outcome.
Methods: We genotyped obese subjects for the dopamine D2 receptors gene (DRD2) utilizing standard PCR techniques. The subjects were assessed for scale weight and for percent body fat using dual energy X-ray absorptiometry (DEXAR). The subjects were divided into matched placebo and CrP groups (400 ug. per day) accordingly. The sample was separated into two independent groups. Those with either an A1/A1 or A1/A2 allele or those with only the A2/A2 allelic pattern. Each of these groups was tested separately for differences between placebo and treatment means for a variety of measures of weight change.
Results: The measures of the change in fat weight (p<0.041), change in body weight (p<0.0 17), the percent change in weight (p<0.044), and the body weight change in kilograms (p<0.012) were all significant for carriers of the DRD2 A2 genotype., whereas no significance was found for any parameter for those subjects possessing a DRD2 A1 allele. Conclusion: These results suggest that the dopaminergic system, specifically the density of the D2 receptors, confers a significant differential therapeutic effect of CrP in terms of weight loss and change in body fat.

This present invention involves developing a weight management nutraceutical that will be customized based upon a polygenetic measurement of a patient's ability to metabolize the nutritional supplementation, as well as contributing factors to their weight and metabolism. Some of those ingredients may include: NUTRIENTS—Calcium citrate (or aspartate) 275 mg, Magnesium citrate (or aspartate) 400 mg, Potassium citrate (or aspartate) 1000 mg, Manganese ascorbate 13.25 mg, B-complex 25 mgs each (B-5 at least 100 mg), L-OptiZinc 20 mg (for insulin, immune and other hormonal pathways); CARBOHYDRATE CRAVING & APPETITE SUPPRESSION—Synaptamine Complex (using DLPA 2000 mg, l-tyrosine 750 mg, 5 HTP 100 mg, Vitamin B6 20 mg and ChromeMate 400 mcg of Cr/day, Rhodiola rosea 200 mg and Rhododendron 100 mg), Citrus aurantium 750 mg, Gymnema sylvestre leaves 25 mg, Hoodia extract 500 mg; ENERGY & METABOLISM—Green Tea Extract—270 mg EGCG, 17-keto-DHEA 50 mg, Banaba extract 100 mg; IMMUNOLOGICAL & ANTI-OXIDANT REPAIR—Bromelain 500 mg (or Papain), Cognizin 750 mg (500 mg minimum effective dose). Protykin 10 mg (antioxidant/bioflavonoid in men; and Phytoestrogen only in women). Gandema lucidum 750 mg; ANTI-STRESS—Passion Flower 75 mg. Magnolia 60 mg; IMMUNE SUPPORT BLEND—Echinacea 50 mg, astragalus, 50 mg, ligustrum, 50 mg, schizandra,50 mg, shitake mushroom 50 mg, Pau D' Arco 50 mg.

The present invention involves a, balanced approach to influencing body recomposition. This balanced approach addresses multiple influencing factors to human metabolism called neurogenobolics which include neurological, genetic, and metabolic. Deficiencies in these interrelated factors contribute to a chronic syndrome negatively impacting body composition. This balanced approach considers multiple factors, including, but not limited to: Neurogenobolic Deficiency Syndrome (NGDS), a poor diet consisting of refined foods, and chronic diseases including frequently co-morbid conditions as cardiovascular disease, diabetes, and obesity.

TABLE 15
NEUROGENOBOLIC HEALTH CHART

| | EFFECTS IN THE BODY | | | | | | | |
|---|---|---|---|---|---|---|---|---|
| | Energy | Fat | Water | Muscle | Bone | Immune System | | |
| CONDITIONS | Levels | Retention | Retention | Density | Density | Health | Stress | Acidosis |
| NGDS | Decrease | Increase | Increase | Decrease | Decrease | Decrease | Increase | Increase |
| (Neurogenobolic | | | | | | | | |
| Deficiency Syndrome) | | | | | | | | |
| Poor Diet | Decrease | Increase | Increase | Decrease | Decrease | Decrease | Increase | Increase |
| Chronic Illness | Decrease | Increase | Increase | Decrease | Decrease | Decrease | Increase | Increase |
| Optimum | Increase | Decrease | Decrease | Increase | Increase | Increase | Decrease | Decrease |
| Health | | | | | | | | |
| Salugen Body | Increase | Decrease | Decrease | Increase | Increase | Increase | Decrease | Decrease |
| Recomposition | | | | | | | | |

This present invention also involves an advanced body recomposition or anti-obesity formulation that would be targeted for patients with diabetes or diabetes-like symptoms, or may be an individual formulation separate from the obesity product. The exact formulation provided to a patient would be individualized based upon some the aforementioned genes related to diabetes and obesity. This formula would include daily amounts of Cinnamon Powder—4000 mg, Banaba Extract—100 mg, No-Pal Cactus—450 mg, Chromium Picolinate 1000 mg, American Ginseng 450 mg, Gymnema Sylvestre 500 mg, Synaptamine complex 2570 mg (Dl-Phenylalanine 2000 mg, L-tyrosine 350 mg, L-glutamine 150 mg, 5-hydroxytryptophan 50 mg, B6 20 mg), Passion flower 100 mg, and HCA-750 mg. There is also the potential utilizing the substance Fenugreek. It is somewhat controversial.

Fenugreek Evidence—Scientists have studied fenugreek for the following health problems:
Diabetes—Early studies suggest that in people with type 2 diabetes (also called non-insulin-dependent or adult-onset diabetes), fenugreek may lower blood sugar levels and may improve problems associated with high blood sugar levels. These problems can include frequent urination, excessive thirst, nerve pain and skin infections. There is one study that suggests fenugreek may also improve blood sugar levels in people with type 1 diabetes (also called insulin-dependent or juvenile diabetes). However, these studies have been small, low quality and not fully convincing. Better studies are underway that may provide more definitive answers. At this time, there is not enough evidence to recommend fenugreek for diabetes. Diabetes is a serious illness and should be treated under the supervision of a qualified health care provider.
High cholesterol—A few studies suggest that fenugreek may help to lower cholesterol levels in the blood, but there is not enough information available at this time to recommend using fenugreek for this purpose.

This present invention includes a business model and methods to utilize DNA and other laboratory tests to customize the formulation of nutritional supplements. This invention also includes methods to provide ongoing monitoring of nutritional supplementation using laboratory tests. As clinically important differences are observed and analyzed, Salugen's business model and methods allow us to adjust an individual's customized nutritional supplementation to address their dynamic health, nutrition, genetic expressions, and metabolism.

This present invention includes a business model and methods to store the DNA samples collected, and genetic measurements taken so that they can be used in algorithms to calculate index scores, which predict future health based upon disease or health progression as well as nutritional supplementation. By aggregating the data and DNA samples collected in a unique nutragenomic data bank, Salugen will be able to determine the probability of response to nutritional supplements and provide ongoing measurement of response to therapy. The tests translate the complex signals of the immune system's multiple genes and pathways, and nutritional metabolites, specifically those associated with healthcare concerns and nutrition, into an objective, actionable score. For example, as this nutragenomic data bank is aggregated and these predictive tests are offered, a patient can know, in advance, how likely they are to respond to the nutritional supplement, as well as correlations with these genetic measurements and their overall health in the future. Salugen testing is a safe, convenient and proven new method to significantly improve a person's quality of life. Rather than having an invasive procedure in a traditional lab, only a simple and quick buccal swab or blood draw is necessary. The sample is sent to the Salugen Laboratory, a state-of-the-art clinical lab, where complex gene information is extracted and analyzed. Using the Salugen algorithms, the information is translated into a single score that distinguishes future response to therapy and healthcare progression. Salugen testing enables patients and their healthcare providers—for the first time—to customize their nutritional supplements, predict their future response, and monitor the immune system early, identifying risks and treatments early. Salugen testing, as the data and samples are aggregated into the Salugen nutragenomic data bank, can provide a longitudinal road map that enables patients and their healthcare providers to determine how a patient's nutritional supplementation is affecting their health. With Salugen's business model, Salugen is the first and only company equipped to not only customize a patient's nutritional supplementation at the onset of taking the supplementation, but further customizing the supplementation as a patient's health changes over time.

SYNAPTOSE—The field of glycomic research is producing advances that promise to increase our understanding of and ability to use complex glycans as therapeutics. Complex glycans located on cell surfaces, deposited in the extracellular matrix and attached to soluble signaling molecules perform crucial roles in the phenotypic expression of cellular genotypes. To date, several hundred genes for glycosylation (attachment of glyconutrients to lipids and proteins facilitated by metalloenzymes) have been identified. Aside from congenital disorders, rate limiting factors of glycosylation include mineral and glyconutrient deficiencies. Aberrant glycosylation produces structural alterations that are categorically characteristic in diseased and dysfunctional cells. Several lines of evidence indicate that the modification of glycoproteins during aberrant or incomplete glycosylation is closely related to a number of neuropathologies, particularly Alzheimer's disease. This present invention utilizes a raw material complex comprised of various glycoforms, called SYNAPTOSE, as a substrate to answer the needs of genetic instructions given to promote competent glycosylation and proteo or lipo-glycogenomic events. Emergence of this new technology represents a capstone to previous research and development from the nutraceutical and pharmaceutical industries. The nutraceutical field has pronounced that neurochemical manipulation leads to well-being in the “era of the brain” and must be addressed via solid scientific evidence based products. Pharmaceutical companies are experiencing increased challenges from the ever growing number of drugs being pulled from the market, promoting the general public's growing and well earned skepticism for, reduced reliance on and loss of confidence in drugs. This erosion of consumer confidence has prompted an expanded search for effective natural remedies with greatly reduced or non-existent negative side effects, a need that the present invention is designed to fulfill.

It is well known that in the brain's reward site, the chemical messenger dopamine works to maintain our normal drives: hunger, thirst, and sex. In fact, dopamine has come to be known as the “pleasure molecule” and/or “anti-stress molecule.” Dopamine transport is facilitated by the glycoprotein “dopamine transporter” (DAT). Research on modified DAT glycosylation shows that non-glycosylated DAT is “misfolded” and less- stable at the cell surface. Non-glycosylated DAT did not transport dopamine as efficiently as wild-type DAT as judged from the sharp reduction in uptake V(max), and prevention of N-glycosylation enhanced the potency of cocaine-like drugs in inhibiting dopamine uptake into intact cells without changing their affinity for DAT when measured in membrane preparations prepared from these cells. Thus, non-glycosylated DAT at the cell surface displays appreciably reduced catalytic activity, impaired cell surface trafficking of dopamine and altered inhibitor sensitivity compared with wild type. When dopamine is released into the synapse, it stimulates a number of dopamine receptors (D1-D5) that result in a feeling of well-being and stress reduction. This is the result of the interaction of numerous transmitters such as serotonin (5HT), endorphins (END), GABA (GB), dopamine (DA), norepinephrine (NE), and acetylcholine (ACH). The process of the interactions at the brain “reward site” is called: the reward cascade. A consensus of the literature suggests that when there is a dysfunction in the “brain reward cascade,” especially in the dopamine system causing a low or hypo-dopaminergic trait, the brain of that person requires a dopamine “fix” to feel good. This high-risk genetic trait leads to multiple drug-seeking behaviors. This is so because alcohol, cocaine, heroin, marijuana, nicotine and glucose all activate release of dopamine, which can heal the abnormal cravings. Moreover, this genetic trait is due to a form of a gene (DRD2A1 allele), which prevents the expression of the normal laying down of dopamine receptors in the brain reward site (Blum et al. 1990). This gene and others involved in neurophysiological processing of the above cited neurotransmitters (i.e. 5HT, END, GB, DA, NE, ACH etc) , have been associated with deficient functions and predispose individuals to have a high risk for addictive, impulsive, and compulsive behavioral propensities such as: severe alcoholism; cocaine, heroin, marijuana, and nicotine addictions; glucose bingeing, pathological gambling, sex addiction, ADHD, Tourette's syndrome, autism, chronic violence, post traumatic stress disorder, schizoid avoidance disorder, conduct disorder, and antisocial behavior. It has been proposed that genetic variants of the D2 dopamine receptor gene and other “reward genes” are important common genetic determinants of the emerging concept coined by Blum as Reward Deficiency Syndrome (RDS). Aberrant glycosylation has been shown to reduce dopamine uptake. Ongoing research involved in chromosomal marking and candidate gene analysis has supported the concept of “polygenic inheritance” and epitasis. While certain pharmaceutical approaches include targeting of single neurotransmitter deficits (e.g. SSRI's), as well as blocking dopaminergic activity to reduce drug effects, our approach includes multiple neuropharmacological targets and enhancement of dopaminergic function as a life-long goal. Gene therapy studies by Nora Volkow revealed that over expression of D2 receptors in the N. accumbens of alcohol drinking rodents results in a significant reduction of both alcohol preference and craving. While the ultimate goal is to early diagnose one's genetic propensity to substance seeking behavior along with potential CNS gene therapy, current diagnosis includes limited non-invasive DNA testing as well as precursor amino-acid-enkephalinase inhibitory therapy. We propose that, based on this previous evidence, substance abuse treatment must involve physiological, psychological, and spiritual modalities. With reference to the physiology, the authors propose a biogenetic model for the diagnosis, treatment, and relapse prevention of RDS behaviors. As proposed herein in this, genotyping, pharmaceutical interventions, nutraceutical therapies such as SYNAPTOSE, neurofeedback, auricular therapy, acupuncture, and chiropractic are discussed as a unifying approach to reduce aberrant cravings and enhance recovery and well being by altering brain chemistry. We also propose that in the future as we continue to obtain scientific support for the involvement of the dopaminergic genes (Dopamine D1 receptor, Dopamine D2 receptor and the dopamine transporter) genotyping, every child born should be genotyped for polymorphisms of the various dopaminergic genes and, based on the genoscore, be provided with variants of our novel SYNAPTOSE formula. For example, epidemiologic studies have already shown that drug dependence is strikingly influenced by genetic factors (h2=0.54). Furthermore, a number of studies have shown the potential benefits of brain chemical manipulation via variants of SYNAPTOSE (see detail description herein).

This present invention supposes that SYNAPTOSE could become an important novel ingredient complex utilized throughout the nutraceutical industry to act alone or as an adjunct to medical foods (e.g. drinks, bars, powders, etc). This present invention believes that SYNAPTOSE will enhance well-being as a novel individual complex, as well as through the Salugen business model and business processes proposed herein. Through DNA-customized versions of SYNAPTOSE, the DNA test results, or genoscore, will provide a true nutritional “gene” therapy tailor made for the individual.

With the rise in proper diagnosis of RDS including ADHD as one subset, and the knowledge that our young children diagnosed with ADHD are particularly prone to Substance Use Disorder we are challenged to prevent a drug abuse epidemic in America. Moreover, 80% of incarcerations are due to drug dependence, whereby 50% of prisoners have been diagnosed with ADHD as well. In addition, since approximately one-third of Americans carry the DRD2 A1 allele, which is associated with a number of RDS behaviors including ADHD and Substance Use Disorder, it is becoming increasing important to genotype patients and consider the impact of RDS.

Synaptose Formula Comprises:

DLPA 2000 mg:

Various Glyconutrients including, but not limited to one or more of the following:
Glucose, fucose, mannose, galactose, xylose, N-acetyl-glucosamine, N-acetyl-galactosamine, N-acetyl-neuraminic acid (sialic acid), all of which can be present as either monosaccharides or complexed as oligo and/or polysaccharides in various foodstuffs including but not limited to: aloe, fenugreek, numerous species of medicinal mushrooms, Western Larch (primarily tree sap and bark), and many more glycoside rich botanical substances. In addition, Arabinose, arabinogalactans, and other polyglycan rich substances will be utilized.
L-Glutamine 150 mg
L-tyrosine 750 mg
5-hydroxytryptophan-100 mg
Chromium (salt) up to 1000 micrograms elemental or more.
Rhodiola rosea 200 mg
Passion flower 100 mg
Vitamin B 6 (20/80) 20 mg
Magnolia flower 20 mg
minerals [calcium (275-750 MG); magnesium (at least 100-750 MG), potassium (at least 250-2000 MG) ] Salts of (−) Hydroxycitric Acid up to 3000 mg

GLYCONUTRIENTS: The Missing Sugars That Heal—This present invention proposes that eight basic sugars represent the essential components of a group of nutrients known as Glyconutrients. The word “Nutraceuticals” was designed to include natural food-based substances having pharmacological-like effects on the human body. First used by the Food and Nutrition Board of the Institute of Medicine, the term was drafted to incorporate all the natural, standardized, non-toxic dietary supplements used in conjunction with improved nutrition. The present invention will also use the term “Glycoceutical” when appropriate. The following definitions may help explain some of this new “Glyco” terminology:
Glyco means “sweet” and, therefore, used when describing a sugar or carbohydrate molecule. “Sugar”, “Carbohydrate” and “Saccharide are all used interchangeably.
Glycoforms (aka Glycoconjugates) are large sugar molecules that combine with proteins and/or fats to cover the surfaces of all cells. These are then known as “glycoproteins” or “glycolipids”.
Glycoproteins are molecules made of sugars and proteins. They are found coating the surface of every cell in the human body that contains a nucleus.
Glycolipids are molecules made of sugars and lipids. “Lipid” and “Fat” are often used interchangeably.
Glyconutrients are the foods and nutritional supplements that provide saccharides along with other glycoforms essential to the body, but which are less than adequate in most diets.

Glyconutrients play a crucial role in almost all aspects of metabolic function. They are key components of the communications circuitry of most molecules. In addition, they are requisite factors in forming the correct three-dimensional structures of molecules and are involved in almost all aspects of molecular bonding, transport and/or interactions. Glycoforms are sensory apparatus that function as cellular recognition and response molecules, primary signaling determinants in metabolic events. Collagen, hemoglobin, lymphocytes (immune complexes) and SOD (four of the five most abundant proteins in the body) are glycoproteins. Flaws in glycosylation (the process of “implanting” glyconutrients in lipids and proteins) form imperfect molecules, impairing metabolic function, are responsible for a wide array of diseases and contribute to aberrant gene expression and obesity (among others). Perfectly structured glycoforms are critically important to competent immune system function. When used along with surgery, chemotherapy, and/or radiation, glyconutrients have proven to help lessen the side effects of these treatments while promoting faster healing and more rapid recovery than the conventional tactics alone. Glycoforms are also essential to the competent workings of the brain and nervous system. Deficiencies in glyconutrients and/or flaws in glycosylation can impair memory and sleep, and foster anxiety, depression and neuropathologies such as Alzheimer's Disease. Impaired glycoprotein and glycolipid synthesis have been implicated in Parkinson's disease. In addition, these abnormalities are associated with an elevation in plasma HMG CoA reductase activity, serum digoxin and dolichol levels, and a reduction in serum magnesium, RBC membrane Na(+)−K+ ATPase activity, and serum ubiquinone levels. Serum tryptophan, serotonin, strychnine, nicotine, and quinolinic acid were elevated, while tyrosine, morphine, dopamine, and noradrenalin were decreased. The total serum glycosaminoglycans (GAG) and glycosaminoglycan fractions (except chondroitin sulphates and hyaluronic acid), the activity of GAG degrading enzymes, carbohydrate residues of serum glycoproteins, the activity of glycohydrolase-beta galactosidase, and serum glycolipids were elevated. HDL cholesterol was reduced and free fatty acids increased. The Red Blood Cell membrane glycosaminoglycans, hexose and fucose residues of glycoproteins and cholesterol were reduced, while phospholipid was increased. The activity of all serum free-radical scavenging enzymes, concentration of glutathione, alpha tocopherol, iron binding capacity, and ceruloplasmin decreased significantly in PD, while the concentration of serum lipid peroxidation products and nitric oxide increased.

In addition, glycoforms have a role in helping the body handle cholesterol and fats, lowering triglycerides and low-density lipoproteins (LDL) and raising the good cholesterol (HDL). Commercials have long touted the benefits of eating oatmeal to bring down cholesterol. What is not mentioned is that it is the sugars (beta-glucans) in oatmeal that are responsible for the beneficial effects.

Another important essential sugar function is to help retain bone density and muscle mass. Research suggests that impaired glycosylation impairs bone protein synthesis and deposition, which contributes to osteopenia, among other disorders. There has been increasing evidence that bone matrix in osteoporosis is accompanied by biochemically-altered collagen, a glycoprotein. The body undergoes wear and tear as it ages. Cells and tissues need to be replaced, remodeled, and renewed continually. Exercise helps the body to develop new blood vessels while increasing muscle mass. Certain kinds of tissues adapt to exercise by increasing the size and number of cells. Adaptation, healing, and recovery are all forms of tissue remodeling. Essential sugars play important roles in these processes.

Scientists have recently discovered that our modern diet is missing some very vital nutrients, and surprisingly enough, these missing nutrients are sugars. After years of research, many scientists believe that the lack of these invaluable sugars in our diet is a major reason for most of today's diseases; even cancer, diabetes, and autoimmune disorders like rheumatoid arthritis, Fibromyalgia, and chronic fatigue syndrome. Today, six out of the top ten causes of death are diet related, and chronic degenerative diseases afflict over 120 million Americans. This present invention proposes that the breakthrough discovery of glyconutrients will have amazing abilities to maintain and balance the immune system, helping to achieve optimum health and preventing even the most insidious of today's killer diseases. Cancer has moved from the tenth leading cause of death to number two, even after Richard Nixon's “War on Cancer” spent thirty billion dollars attempting to find a cure. Diabetes has increased 700 percent since 1959. Nearly fifteen million American adults suffer from asthma and the Environmental Health Commission predicts that number will increase to twenty-nine million by 2020. Twenty-one million Americans suffer from arthritis, and approximately fifty million Americans suffer from autoimmune diseases, with 75 percent of these being female. Many of these autoimmune conditions were practically non-existent only 30 years ago.

The average diet of children today routinely includes soft drinks, processed cereal, pizza, candy, fast food and their favorite and often only source of vegetables: French fries. Lifestyle activities like this are contributing to the dramatic rise in ADHD, to the point where eight million American children need to be drugged daily? Autism has gone from 1 in 10,000 children to 1 in 150 in just ten years. Further, adult-onset diabetes is occurring at epidemic rates in children as young as eight.

Complete glyconutrition provides healthy immune system function. Glyconutritionals (glycoceuticals) are very unique because they are immune system modulators. This means glyconutrient supplementation can help correct an overactive immune system (auto-immune diseases), boost an under active immune system (chronic or recurring infections), and keep immune armies in tip-top shape for exceptional disease correction and/or prevention.

Scientific Research Backing Glyconutrition—There are over 20,000 studies conducted annually on glycoforms alone. Researchers from universities and major pharmaceutical companies realize the importance of this new discovery. Breaking the “sugar-code” will mean a tremendous advancement in health and medicine. Studies confirm that the eight essential biologically active sugars can accomplish amazing results. The following are just a few examples of the exciting possibilities of Glyconutrition:
Dramatically raises natural killer cell and macrophage count against infectious organisms.
Activates immune T-cell activity only when invaders or antigens are present.
Decreases cell death in chronic fatigue syndrome.
Dramatically elevates disease resistance in weakened individuals.
Acts as antioxidant compounds, which boost the collection of dangerous free radicals.
Protects the body against toxin and pollutant exposure.
Slows premature aging.
Decreases inflammation in diseases like rheumatoid arthritis.
Helps immune cells recognize invaders due to a mutual “sugar exchange” of information.
Enables cellular components to stick to each other initiating the right reactions.

In addition, glycoforms have a role in helping the body handle cholesterol and fats, lowering triglycerides and low-density lipoproteins (LDL) and raising the good cholesterol (HDL). Commercials have long touted the benefits of eating oatmeal to bring down cholesterol. What is not mentioned is that it is the sugars (beta-glucans) in oatmeal that are responsible.

Another important essential sugar function is to help retain bone density and muscle mass. The body undergoes wear and tear as it ages. Cells and tissues need to be replaced, remodeled, and renewed continually. Exercise helps the body to develop new blood vessels while increasing muscle mass. Certain kinds of tissues adapt to exercise by increasing the size and number of cells. Adaptation, healing, and recovery are all forms of tissue remodeling. Essential sugars play important roles in these processes.

Scientists have recently discovered that our modern diet is missing some very vital nutrients, and surprisingly enough, these missing nutrients are sugars. After years of research, many scientists believe that the lack of these invaluable sugars in our diet is a major reason for most of today's diseases; even cancer, diabetes, and autoimmune disorders like rheumatoid arthritis, fibromyalgia, and chronic fatigue syndrome. Today, six out of the top ten causes of death are diet related, and chronic degenerative diseases afflict over 120 million Americans. This present invention proposes that the breakthrough discovery of glyconutrients will have amazing abilities to maintain and balance your immune system, helping you to achieve optimum health and prevent even the most insidious of today's killer diseases. Cancer has moved from the tenth leading cause of death to number two, even after Richard Nixon's “War on Cancer” spent thirty billion dollars attempting to find a cure. Diabetes has increased 700 percent since 1959. Nearly fifteen million American adults suffer from asthma and the Environmental Health Commission predicts that number will increase to twenty-nine million by 2020. Twenty-one million Americans suffer from arthritis, and approximately fifty million Americans suffer from autoimmune diseases, with 75 percent of these being female. Many of these autoimmune conditions were practically non-existent only 30 years ago.

Look at the average diet of children today—soft drinks, processed cereal, pizza, candy, fast food and their favorite and often only source of vegetables: French fries. Could this be why we are seeing a dramatic rise in ADHD, to the point where eight million American children need to be drugged daily? Autism has gone from 1 in 10,000 children to 1 in 150 in just ten years. Further, adult-onset diabetes is occurring at epidemic rates in children as young as eight.

Complete Glyconutrition provides immune balance, fortification, and maintenance. Glycoceuticals are very unique because they are immune system modulators. This means glyconutrient supplementation can help to correct an overactive immune system (auto-immune diseases), boost an under active immune system (chronic or recurring infections), and keep immune armies in tip-top shape for exceptional disease prevention.

Scientific Research Backing Glyconutrition—There are over 20,000 studies conducted annually on glycoforms alone. Researchers from universities and major pharmaceutical companies realize the importance of this new discovery. Breaking the “sugar-code” will mean a tremendous advancement in health and medicine. Studies confirm that the eight essential biologically active sugars can accomplish amazing results. The following are just a few examples of the exciting possibilities of Glyconutrition:
Dramatically raises natural killer cell and macrophage count against infectious organisms.
Activates immune T-cell activity only when invaders or antigens are present.
Decreases cell death in chronic fatigue syndrome.
Dramatically elevates disease resistance in weakened individuals.
Acts as antioxidant compounds, which boost the collection of dangerous free radicals.
Protects the body against toxin and pollutant exposure.
Slows premature aging.
Decreases inflammation in diseases like rheumatoid arthritis.
Helps immune cells recognize invaders due to a mutual “sugar exchange” of information.
Enables cellular components to stick to each other initiating the right reactions.

While in the foregoing specification this invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

We claim:

1. A composition for measuring and treating genetic (DNA) and metabolomic factors affecting disease states comprising at least one of the following:
an herbal component;
a vitamin component;
a mineral component; and
a homeopathic component.

2. The composition of claim 1 for measuring and treating genetic and metabolomic factors of at least one of the following disease states: cardiovascular, diabetes, dysrthymias, lipid response, nicotine dependence, hypertension, oxidant stress, inflammation, carbohydrate metabolism, obesity including Achondoplasia, Adiposis dolorosa, Posterior polymiphous corneal dystrophy, Momo Syndrome, Prader-Willi syndrome, Schinzel syndrome, Polycystic ovarian syndrome, Alstrom Syndrome, Badet-Biedi, Biermond syndrome, Cohen syndrome, Cushing Syndrome, Pickwickian syndrome, Short stature-obesity syndrome, Summit syndrome, Borjeson-Forssman-Lehmann syndrome, Chroideremia with deafness & obesity, Wilson-Turner syndrome, central nervous system disorders such as cognition and memory deficits, Neurogenobolic Deficiency Syndrome (NGDS), Reward Deficiency Syndrome (RDS), anxiety, autoimmune disease, Rheumatoid arthritis including all forms as defined herein: Achilles tendonitis, Achondroplasia, Acromegalic arthropathy, Adhesive capsulitis, Adult onset Still's disease, Ankylosing spondylitis, Anserine bursitis, Avascular necrosis, Behcet's syndrome, Bicipital tendonitis, Blount's disease, Brucellar spondylitis, Bursitis, Calcaneal bursitis, Calcium pyrophosphate dihydrate (CPPD), Crystal deposition disease, Caplan's syndrome, Carpal tunnel syndrome, Chondrocalcinosis, Chondromalacia patellae, Chronic synovitis, Chronic recurrent multifocal osteomyelitis, Churg-Strauss syndrome, Cogan's syndrome, Corticosteroid-induced osteoporosis, Costosternal syndrome, CREST syndrome, Cryoglobulinemia, Degenerative joint disease, Dermatomyositis, Diabetic finger sclerosis, Diffuse idiopathic skeletal hyperostosis (DISH), Discitis, Discoid lupus erythematosus, Drug-induced lupus, Duchenne's muscular dystrophy, Dupuytren's contracture, Ehlers-Danlos syndrome, Enteropathic arthritis, Epicondylitis, Erosive inflammatory osteoarthritis, Exercise-induced compartment syndrome, Fabry's disease, Familial Mediterranean fever, Farber's lipogranulomatosis, Felty's syndrome, Fibromyalgia, Fifth's disease, Flat feet, Foreign body synovitis, Freiberg's disease, Fungal arthritis, Gaucher's disease, Giant cell arteritis, Gonococcal arthritis, Goodpasture's syndrome, Gout, Granulomatous arteritis, Hemarthrosis, hemochromatosis, Henoch-Schonlein purpura, Hepatitis B surface antigen disease, Hip dysplasia, Hurler syndrome, Hypermobility syndrome, Hypersensitivity vasculitis, Hypertrophic osteoarthropathy, Immune complex disease, Impingement syndrome, Jaccoud's arthropathy, Juvenile ankylosing spondylitis, Juvenile dermatomyositis, Juvenile rheumatoid arthritis, Kawasaki disease, Kienbock's disease, Legg-Calve-Perthes disease, Lesch-Nyhan syndrome, Linear scleroderrna, Lipoid dermatoarthritis, Lofgren's syndrome, Lyme disease, Malignant synovioma, Marfan's syndrome, Medial plica syndrome, Metastatic carcinomatous arthritis, Mixed connective tissue disease (MCTD), Mixed cryoglobulinemia, Mucopolysaccharidosis, Multicentric reticulohistiocytosis, Multiple epiphyseal dysplasia, Mycoplasmal arthritis, Myofascial pain syndrome, Neonatal lupus, Neuropathic arthropathy, Nodular panniculitis, Ochronosis, Olecranon bursitis, Osgood-Schlatter's disease, Osteoarthritis, Osteochondromatosis, Osteogenesis imperfecta, Osteomalacia, Osteomyelitis, Osteonecrosis, Osteoporosis, Overlap syndrome, Pachydermoperiostosis Paget's disease of bone, Palindromic rheumatism, Patellofemoral pain syndrome, Pellegrini-Stieda syndrome, Pigmented villonodular synovitis, Piriformis syndrome, Plantar fasciitis, Polyarteritis nodosa, Polymyalgia rheumatica, Polymyositis, Popliteal cysts, Posterior tibial tendonitis, Pott's disease, Prepatellar bursitis, Prosthetic joint infection, Pseudoxanthoma elasticum, Psoriatic arthritis, Raynaud's phenomenon, Reactive arthritis/Reiter's syndrome, Reflex sympathetic dystrophy syndrome, Relapsing polychondritis, Retrocalcaneal bursitis, Rheumatic fever, Rheumatoid arthritis, Rheumatoid vasculitis, Rotator cuff tendonitis, Sacroiliitis, Salmonella osteomyelitis, Sarcoidosis, Saturnine gout, Scheuermann's osteochondritis, Scleroderma, Septic arthritis, Seronegative arthritis, Shigella arthritis, Shoulder-hand syndrome, Sickle cell arthropathy, Sjogren's syndrome, Slipped capital femoral epiphysis, Spinal stenosis, Spondylolysis, Staphylococcus arthritis, Stickler syndrome, Subacute cutaneous lupus, Sweet's syndrome, Sydenham's chorea, Syphilitic arthritis, Systemic lupus erythematosus (SLE), Takayasu's arteritis, Tarsal tunnel syndrome, Tennis elbow, Tietse's syndrome, Transient osteoporosis, Traumatic arthritis, Trochanteric bursitis, Tuberculosis arthritis, Arthritis of Ulcerative colitis, Undifferentiated connective tissue syndrome (UCTS), Urticarial vasculitis, Viral arthritis, Wegener's granulomatosis, Whipple's disease, Wilson's disease, and Yersinial arthritis.

3. The composition of claim 1, wherein an effective amount of the herbal component for Joint Health comprises at least one of the following: Black Currant Oil; Black Currant Seed Oil; Ribes nigrum; Borage Oil; Borage Seed Oil; Borago officinalis; Bovine Cartilage; Bromelain; Ananas comosus; Cat's Claw; Uncaria tomentosa; Cetyl Myristoleate; Cetyl-M; Cis-9cetylmyristoleate; Cmo; Chondroitin Sulfate; Collagen Hydrolysate; Collagen; Gelatin; Gelatine; Gelatin Hydrolysate; Hydrolyzed [Denatured] Collagen; Devil's Claw; Devil's Claw Root; Grapple Plant; Wood Spider; Harpagophytum procumbens; Dhea-Dehydroepiandrosterone; Dmso-Dimethyl Sulfoxide; Evening Primrose Oil; Evening Primrose; Primrose; Oenothera biennis; other Oenothera species; Feverfew; Tanacetum parthenium; Fish Oil; Flaxseed; Flaxseed Oil; Flax Oil; Linseed Oil; Linum usitatissimum; Ginger; Zingiber officinale; Gingko; Gingko biloba; Ginseng; American ginseng; panax quinquefolius; Asian ginseng; panax ginseng; Siberian ginseng; eleutherococcus senticosus; Gla (Gamma-Linolenic Acid); Glucosamine; Glucosamine sulfate; glucosamine hydrochloride; N-acetyl glucosamine; Gotu Kola; Gotu Cola; Brahmi; Brahma-Buti; Indian Pennywort; Centella asiatica; Grapeseed; Grapeseed Oil; Grapeseed Extract; Vitis vinifera; Green Tea; Chinese Tea; Camellia sinensis; Guggul; Gugulipid; Guggal; Commiphora mukul; Indian Frankincense; Frankincense; Boswellia; Boswellin; Salai Guggal; Boswellia serrata; Kava Kava; Kava; Kava Pepper; Tonga; Kava Root; Piper methysticum; Melatonin; Msm (Methylsulfonylmethane); New Zealand Green-Lipped Mussel; Perna Canaliculus; Phellodendron Amurense; Sam-E (S-adenosyl-L-methione); Shark Cartilage; Cartilage; St. John's Wort; Hypercium perforatum; Stinging Nettle; Urtica dioica; Thunder God Vine; Tripterygium wilfordii; Turmeric; Curcuma longa; Curcuma domestica; Type II Undenatured Chicken Collagen; Chicken Collagen; Chicken Type II Collagen; Type II Collagen; Valerian; Valeriana officianalis; White Willow; Willow Bark; Salix Alba; White Willow Bark; Wild Yam; Discorea villosa; Ganoderma Lucidum; Mangosteen Extract; and Quercetin.

4. The composition of claim 3, wherein the herbal component ranges from approximately 1 mcg to 100,000 mg in a daily therapeutic administration.

5. The composition of claim 1, wherein an effective amount of the vitamin component for Joint Health comprises at least one of the following: Folic Acid; Vitamin C; and Vitamin B₆.

6. The composition of claim 1, wherein an effective amount of the mineral component for Joint Health comprises at least one of the following: manganese; potassium; magnesium; calcium; coral calcium; Sierasil®; Algae Cal® and any active salt thereof.

7. The composition of claim 1, wherein an effective amount of the homeopathic component for Joint Health comprises at least one of the following: Aceonite 12X; Belladonna 12X; Bryonia 12X; Chamonlia 6X; Ferrum Phos 12X; Gelsemium 12X; and Berberis 6X.

8. The composition of claim 1 further comprises at least one of the following: an opiate destruction-inhibiting substance; a neurotransmitter synthesis precursor; a tryptohan enhancing substance; an catecholoamine-O-methyl transferase (COMT) inhibitor; and an acetylcholinase/cholinesterase inhibitor.

9. The composition of claim 8, wherein the opiate destruction inhibiting substance comprises at least one of the following: D-phenylalanine; D-Leucine; any D-amino acid; and hydrocinnamic acid.

10. The composition of claim 8, wherein the neurotransmitter synthesis precursor comprises at least one of the following: dopamine precursors L-Tyr, L-Phe, and L-dopa; serotonin precursors L-Trp and 5-hydroxytryptophan; gamma amino butyric acid (GABA) precursors L-glutamine, L-glutamic acid and L-glutamate; acetylcholine (ACH) and acetylcarnitine precursors L-choline and L-acetylcholine; L-carnitine; and aceytlcarnitine.

11. The composition of claim 8, wherein the tryptophan enhancing substance comprises at least one of the following chromium salts: picolinate, polyicotinate, chloride, and any active salts thereof.

12. The composition of claim 1, wherein the at least one other component comprises at least one of the following: Rhodiola (rosea extract) and huperazine (A).

13. A method for measuring genetic (DNA) and metabolomic contributing factors affecting disease states which comprises the steps of:
Collecting DNA;
Processing, measuring and analyzing genes of the collected DNA;
Identifying any mutations of the collected DNA;
Using a custom algorithm to obtain an index score;
Formulating a composition based upon the identified index score; and
Administering to a human the custom composition for treatment of any identified mutations or disease states.

14. The method of claim 13, wherein the collecting the DNA comprises at least one of the following: using a buccal swab, obtaining a whole blood sample, and other collection method.

15. The method of claim 13, wherein the measuring of the DNA comprises at least one of the following methods: Elisa, TaqMan, PCR, and Invader.

16. The method of claim 13, wherein the identifying mutations comprises measuring multiple genetic mutations through single nucleotide polymorphisms, gene expression, or other forms of genetic and phenotypic measurement for the purposes of customizing or adjusting the formulation of nutritional supplements.

17. The method of claim 13, wherein the custom algorithm comprises measuring two genes through single nucleotide polymorphisms and combining genetic mutations into index scores to represent specific pre-defined formulations.

18. The method of claim 13, wherein the index score comprises a value related to the number of identified mutations.

19. The method of claim 18, wherein the index score is 0 for no identified mutations, 1 for an identified mutation on a gene, 2 for an identified mutation on a second gene, and 3 for an identified mutation on two separate genes.

20. The method of claim 13, wherein using a custom algorithm to obtain an index score comprises providing a understandable, simple report to a patient and clinician for providing insight into disease diagnosis, stratification, and prognosis.

21. The method of claim 13, wherein the disease state comprises at least one of the following: joint health involving reducing pain, inflammation, and joint damage; stress and anxiety relief; preventing sleep loss and insomnia; combating obesity and promoting lipid reduction; lethargy or lack of energy; skin, hair, and nail health; overall mental health and well-being; reducing the signs and symptoms of attention deficit hyperactivity disorder; reducing the signs and symptoms of depression; reducing the signs and symptoms of pre-menstrual dysphorric disorder; and, overcoming the dependence and urges of smoking, alcoholism, and drug dependence.

22. The method of claim 13 for formulating and administering the custom composition for treatment of at least one of the following disease states: cardiovascular, diabetes, dysrthymias, lipid response, nicotine dependence, hypertension, oxidant stress, inflammation, carbohydrate metabolism, obesity including Achondoplasia, Adiposis dolorosa, Posterior polymiphous corneal dystrophy, Momo Syndrome, Prader-Willi syndrome, Schinzel syndrome, Polycystic ovarian syndrome, Alstrom Syndrome, Badet-Biedi, Biermond syndrome, Cohen syndrome, Cushing Syndrome, Pickwickian syndrome, Short stature-obesity syndrome, Summit syndrome, Borjeson-Forssman-Lehmann syndrome, Chroideremia with deafness & obesity, Wilson-Tumer syndrome, central nervous system disorders such as cognition and memory deficits, Neurogenobolic Deficiency Syndrome (NGDS), Reward Deficiency Syndrome (RDS) anxiety, autoimmune disease, Rheumatoid arthritis including all forms as defined herein: Achilles tendonitis, Achondroplasia, Acromegalic arthropathy, Adhesive capsulitis, Adult onset Still's disease, Ankylosing spondylitis, Anserine bursitis, Avascular necrosis, Behcet's syndrome, Bicipital tendonitis, Blount's disease, Brucellar spondylitis, Bursitis, Calcaneal bursitis, Calcium pyrophosphate dihydrate (CPPD), Crystal deposition disease, Caplan's syndrome, Carpal tunnel syndrome, Chondrocalcinosis, Chondromalacia patellae, Chronic synovitis, Chronic recurrent multifocal osteomyelitis, Churg-Strauss syndrome, Cogan's syndrome, Corticosteroid-induced osteoporosis, Costostemal syndrome, CREST syndrome, Cryoglobulinemia, Degenerative joint disease, Dermatomyositis, Diabetic finger sclerosis, Diffuse idiopathic skeletal hyperostosis (DISH), Discitis, Discoid lupus erythematosus, Drug-induced lupus, Duchenne's muscular dystrophy, Dupuytren's contracture, Ehlers-Danlos syndrome, Enteropathic arthritis, Epicondylitis, Erosive inflammatory osteoarthritis, Exercise-induced compartment syndrome, Fabry's disease, Familial Mediterranean fever, Farber's lipogranulomatosis, Felty's syndrome, Fibromyalgia, Fifth's disease, Flat feet, Foreign body synovitis, Freiberg's disease, Fungal arthritis, Gaucher's disease, Giant cell arteritis, Gonococcal arthritis, Goodpasture's syndrome, Gout, Granulomatous arteritis, Hemarthrosis, hemochromatosis, Henoch-Schonlein purpura, Hepatitis B surface antigen disease, Hip dysplasia, Hurler syndrome, Hypermobility syndrome, Hypersensitivity vasculitis, Hypertrophic osteoarthropathy, Immune complex disease, Impingement syndrome, Jaccoud's arthropathy, Juvenile ankylosing spondylitis, Juvenile dermatomyositis, Juvenile rheumatoid arthritis, Kawasaki disease, Kienbock's disease, Legg-Calve-Perthes disease, Lesch-Nyhan syndrome, Linear scleroderma, Lipoid dermatoarthritis, Lofgren's syndrome, Lyme disease, Malignant synovioma, Marfan's syndrome, Medial plica syndrome, Metastatic carcinomatous arthritis, Mixed connective tissue disease (MCTD), Mixed cryoglobulinemia, Mucopolysaccharidosis, Multicentric reticulohistiocytosis, Multiple epiphyseal dysplasia, Mycoplasmal arthritis, Myofascial pain syndrome, Neonatal lupus, Neuropathic arthropathy, Nodular panniculitis, Ochronosis, Olecranon bursitis, Osgood-Schlatter's disease, Osteoarthritis, Osteochondromatosis, Osteogenesis imperfecta, Osteomalacia, Osteomyelitis, Osteonecrosis, Osteoporosis, Overlap syndrome, Pachydermoperiostosis Paget's disease of bone, Palindromic rheumatism, Patellofemoral pain syndrome, Pellegrini-Stieda syndrome, Pigmented villonodular synovitis, Piriformis syndrome, Plantar fasciitis, Polyarteritis nodosa, Polymyalgia rheumatica, Polymyositis, Popliteal cysts, Posterior tibial tendonitis, Pott's disease, Prepatellar bursitis, Prosthetic joint infection, Pseudoxanthoma elasticum, Psoriatic arthritis, Raynaud's phenomenon, Reactive arthritis/Reiter's syndrome, Reflex sympathetic dystrophy syndrome, Relapsing polychondritis, Retrocalcaneal bursitis, Rheumatic fever, Rheumatoid arthritis, Rheumatoid vasculitis, Rotator cuff tendonitis, Sacroiliitis, Salmonella osteomyelitis, Sarcoidosis, Saturnine gout, Scheuermann's osteochondritis, Scleroderma, Septic arthritis, Seronegative arthritis, Shigella arthritis, Shoulder-hand syndrome, Sickle cell arthropathy, Sjogren's syndrome, Slipped capital femoral epiphysis, Spinal stenosis, Spondylolysis, Staphylococcus arthritis, Stickler syndrome, Subacute cutaneous lupus, Sweet's syndrome, Sydenham's chorea, Syphilitic arthritis, Systemic lupus erythematosus (SLE), Takayasu's arteritis, Tarsal tunnel syndrome, Tennis elbow, Tietse's syndrome, Transient osteoporosis, Traumatic arthritis, Trochanteric bursitis, Tuberculosis arthritis, Arthritis of Ulcerative colitis, Undifferentiated connective tissue syndrome (UCTS), Urticarial vasculitis, Viral arthritis, Wegener's granulomatosis, Whipple's disease, Wilson's disease, or Yersinial arthritis.

23. The method of claim 13, wherein the composition administered comprises at least one of the following: an herbal component; a vitamin component; a mineral component; and a homeopathic component.

24. The composition of claim 23, wherein an effective amount of the herbal component for Joint Health comprises at least one of the following: Black Currant Oil; Black Currant Seed Oil; Ribes nigrum; Borage Oil; Borage Seed Oil; Borago officinalis; Bovine Cartilage; Bromelain; Ananas comosus; Cat's Claw; Uncaria tomentosa; Cetyl Myristoleate; Cetyl-M; Cis-9cetylmyristoleate; Cmo; Chondroitin Sulfate; Collagen Hydrolysate; Collagen; Gelatin; Gelatine; Gelatin Hydrolysate; Hydrolyzed [Denatured] Collagen; Devil's Claw; Devil's Claw Root; Grapple Plant; Wood Spider; Harpagophytum procumbens; Dhea-Dehydroepiandrosterone; Dmso-Dimethyl Dimethyl Sulfoxide; Evening Primrose Oil; Evening Primrose; Primrose; Oenothera biennis; other Oenothera species; Feverfew; Tanacetum parthenium; Fish Oil; Flaxseed; Flaxseed Oil; Flax Oil; Linseed Oil; Linum usitatissimum; Ginger; Zingiber officinale; Gingko; Gingko biloba; Ginseng; American ginseng; panax quinquefolius; Asian ginseng; panax ginseng; Siberian ginseng; eleutherococcus senticosus; Gla (Gamma-Linolenic Acid); Glucosamine; Glucosamine sulfate; glucosamine hydrochloride; N-acetyl glucosamine; Gotu Kola; Gotu Cola; Brahmi; Brahma-Buti; Indian Pennywort; Centella asiatica; Grapeseed; Grapeseed Oil; Grapeseed Extract; Vitis vinifera; Green Tea; Chinese Tea; Camellia sinensis; Guggul; Gugulipid; Guggal; Commiphora mukul; Indian Frankincense; Frankincense; Boswellia; Boswellin; Salai Guggal; Boswellia serrata; Kava Kava; Kava; Kava Pepper; Tonga; Kava Root; Piper methysticum; Melatonin; Msm (Methylsulfonylmethane); New Zealand Green-Lipped Mussel; Perna Canaliculus; Phellodendron Amurense; Sam-E (S-adenosyl-L-methione); Shark Cartilage; Cartilage; St. John's Wort; Hypercium perforatum; Stinging Nettle; Urtica dioica; Thunder God Vine; Tripterygium wilfordii; Turmeric; Curcuma longa; Curcuma domestica; Type II Undenatured Chicken Collagen; Chicken Collagen; Chicken Type II Collagen; Type II Collagen; Valerian; Valeriana officianalis; White Willow; Willow Bark; Salix Alba; White Willow Bark; Wild Yam; Discorea villosa; Ganoderma Lucidum; Mangosteen Extract; and Quercetin.

25. The composition of claim 24, wherein the herbal component ranges from approximately 1 mcg to 100,000 mg in a daily therapeutic administration.

26. The composition of claim 23, wherein an effective amount of the vitamin component for Joint Health comprises at least one of the following: Folic Acid; Vitamin C; and Vitamin B₆.

27. The composition of claim 23, wherein an effective amount of the mineral component for Joint Health comprises at least one of the following: manganese; potassium; magnesium; calcium; coral calcium; Sierasil®; Algae Cal® and any active salt thereof.

28. The composition of claim 23, wherein an effective amount of the homeopathic component for Joint Health comprises at least one of the following: Aceonite 12X; Belladonna 12X; Bryonia 12X; Chamonlia 6X; Ferrum Phos 12X; Gelsemium 12X; and Berberis 6X.

29. The composition of claim 23, further comprises at least one of the following: an opiate destruction-inhibiting substance; a neurotransmitter synthesis precursor; a tryptohan enhancing substance; an catecholoamine-O-methyl transferase (COMT) inhibitor; and an acetylcholinase/cholinesterase inhibitor.

30. The composition of claim 29, wherein the opiate destruction inhibiting substance comprises at least one of the following: D-phenylalanine; D-Leucine; any D-amino acid; and hydrocinnamic acid.

31. The composition of claim 29, wherein the neurotransmitter synthesis precursor comprises at least one of the following: dopamine precursors L-Tyr, L-Phe, and L-dopa; serotonin precursors L-Trp and 5-hydroxytryptophan; gamma amino butyric acid (GABA) precursors L-glutamine, L-glutamic acid and L-glutamate; acetylcholine (ACH) and acetylcarnitine precursors L-choline and L-acetylcholine; L-carnitine; and aceytlcarnitine.

32. The composition of claim 29, wherein the tryptophan enhancing substance comprises at least one of the following chromium salts: picolinate, polyicotinate, chloride, and any active salts thereof.

33. The composition of claim 23, wherein the at least one other component comprises at least one of the following: Rhodiola (rosea extract) and huperazine (A).

34. The method of claim 13, wherein the composition administered comprises Synaptamine™ in a daily therapeutic administration from approximately: 32-10,000 mg of Dl-phenylalanine, 10-10,000 mg of l-tyrosine, 5-5,000 mg of l-tryptophan, 3-30,000 mg of L-glutamine, 2-30,000 mcg of chromium salt, 1-300 mg of pyridoxal-5′-phosphate, and 1-10,000 mg Rhodiola rosea.

35. The composition of claim 34 further comprises: 5-15,000 mg bromelian, 2400GDU/gm, 5-10,000 mg boswellia extract (65% boswellic acid), 5-20,000 mg Chondroitin Sulfate, 1-10,000 mcg folic acid, 2-20,000 mg Ganoderma Lucidium, 2-20,000 Glucosamine Sulfate, 3-3000 mg Hyaluronic Acid, 3-5,000 Mangosteen Extract (40% gamma-mangostin), 3-5,000 mg Quercetin, 1-1,000 mg Manganese, and 10-50,000 mg Vitamin C in a daily therapeutic administration.

36. The composition of claim 35 further comprises 5-10,000 mg SieraSil® (Siera Mountain Minerals, Vanvouver, BC) in a daily therapeutic administration.

37. The composition of claim 35 further comprises 5-10,000 mg AlgaeCal® (Algaecal International, Las Vegas, Nev.) in a daily therapeutic administration.

38. The composition of claim 35 further comprises 5-10,000 mg Coral Calcium (Marine Bio Tokoyo, Japan) in a daily therapeutic administration.

39. The method of claim 13, wherein the disease state being treated comprises Neurogenobolic Deficiency Syndrome (NGDS), including obesity, carbohydrate craving, increase lipid storage, unwanted weight gain, low energy, low metabolic rate, compromised immune response and anti-oxidant repair, high stress and a high cortisol level.

40. The method of claim 39, wherein the composition administered for comprises Synaptamine™ in a daily therapeutic administration from approximately: 32-10,000 mg of Dl-phenylalanine, 10-10,000 mg of l-tyrosine, 5-5,000 mg of l-tryptophan, 3-30,000 mg of L-glutami 2-30,000 mcg of chromium salt, 1-300 mg of pyridoxal-5′-phosphate, and 1-10,000 mg Rhodiola rosea.

41. The method of claim 40, wherein the composition administered further comprises: 5-2000 mg of Calcium citrate or aspartate; 5-2000 mg of Magnesium citrate or aspartate; 10-3,000 mg Calcium citrate or aspartate, 5-2000 mg Potassium citrate or aspartate; 5-2000 mg, Manganese ascorbate; 1-2000 mg B-complex (B-5); 2-1500 mg L-OptiZinc; 5-2000 mg Rhododendron; 2-3000 mg Citrus aurantium; 2-2000 mg Gymnema sylvestre leaves; 10-3000 mg Hoodia extract; 2-3000 mg Green Tea Extract-EGCG; 5-2000 mg 17-keto-DHEA; 5-2000 mg Banaba extract; 5-4000 mg Bromelain or Papain; 2-3000 mg Protykin; 5-10,0000 mg Gandema Lucidum; 2-5,000 mg-Passion Flower; 2-2000 mg Magnolia; 2-2000 mg Echinacea; 2-2000 mg astragulus; 2-2000 mg ligustrum; 2-2000 mg schizandra; 2-2000 mg shitake mushroom; 2-2000 mg Pau D'Arco.

42. The method of claim 39, wherein the composition administered comprises: 5-5,000 mg carnitine, 2.5-2,500 mg acetylcarnitine, and 5-20,000 mg (−)—Hydroxycitric acid (HCA), and 5-2000 mg desnutrin in a daily therapeutic administration.

43. The method of claim 13, wherein the disease state being treated comprises all forms of diabetes including Type II diabetes.

44. The method of claim 43, wherein the composition administered comprises: 5-10,000 mg Cinnamin Powder; 5-5,000 mg Banaba Extract; 2-6,000 mg No-Pal Cactus; 20-30,000 mcg Chromium Picolinate; 5-10,000 mg American Ginseng; 5-5,000 mg Gymnea Sylvestre; 2770 mg Synapatamine complex comprising: Dl-Phenylalanine 2000 mg, L-tyrosine 350 mg, L-glutamine 150 mg, 5-hydroxytryptophan 50 mg, B6 20 mg, and Rhodiola rosea 200 mg; 2-2000 mg Passion flower; 5-20,000 mg HCA; and 5-1000 mg Fenugreek.

45. The method of claim 43, wherein the composition administered comprises at least one of the following: 5-2000 mg Vanadyl Sulfate; 2-3000 mg Bitter Melon Extract; 10-1500 mg Bilberry Extract; 10-2500 mg Jambolan; 2-3000 mg Pterocarpus Marsupium; and 5-6000 mg Gulvel in a daily therapeutic administration.

46. The method of claim 13, wherein the disease state being treated comprises stress/anxiety including posttraumatic stress disorder and other known anxiety disorders.

47. The method of claim 46, wherein the composition administered comprises: 2-2000 mg Passion flower; 5-1500 mg Kava Kava; 5-10,000 mg Rhodiola rosea; 5-10,000 mg Rhodendron; 5-10,000 mg dl-phenylalanine; 2-5000 mg l-tyrosine; 10-5,000 mg L-glutamine; 5-2000 mg 5-Hyroxytryptophane; 20-30,000 mcg Chromium Picolonate or other active salt thereof; 1-1000 mg Pyridoxyl phosphate; 1-1000 mg Vitamin B complex; 5-2000 mg Calcium citrate; 5-2000 mg Magnesium acscorbate; 10-20,000 mg Hydroxycitric acid; and 2-2000 mg Magnolia.

48. The method of claim 13, wherein the composition administered comprises Neurotensin for the relief of pain and food cravings.

49. The method of claim 13, wherein the genes of the collected DNA processed, measured and analyzed comprise at least one of the following: polymorphisms in Beta-adrenergic receptors; angiotensin converting enzyme (ACE) gene polymorphisms; angiotensin 11 T1 receptor gene polymorphisms; polymorphisms in the gene that controls the enzyme cholesteryl ester transfer protein; potassium channel mutations; polymorphisms in cytochrome P-450 enzymes including CYP2D6; genetic mutation in a protein product of the HER2/neu oncogene; polymorphisms of the C825T gene involved in second messenger G-protein {beta}3; genetic variation of the apolipoprotein constituents of the lipoprotein molecules (APOE gene locus); variation of the CT and TT allele of the dopamine D2 receptor gene; a SNP (polymorphism) designated AA, at nucleotide position-6 of the ANG gene; Apo-A1 gene; Methylene Tetrahydrofolate Reductase (MTHFR) including the C677T polymorphism of this gene; polymorphisms in the proinflammatory cytokine tumor necrosis factor (TNF); polymorphisms in the carbohydrate responsive element-binding protein (ChREBP) gene; C polymorphisms of the Leptin receptor gene (Leptin Gene and Leptin Receptor Gene-R109R homozygotes, LEP A19G polymorphism and the LEPR 109R carriers); polymorphisms of the dopamine D2 receptors gene (DRD2); polymorphisms of the dopamine D1,D3, D4,D5 genes; dopamine D2 receptor polymorphisms Ser311cys and Taq1A; c-fos; c-jun and c-myc; : Sterol Regulatory Element Protein-i (SREBP-1c); mitochondrial glycerol-3-phosphate aceyltransferase gene (MGPAT) and the peroxisome proloferator-activated receptor (PPAR-gamma-2;. Pro12Ala polymorphism of PPARgamma gene; Tryptophan 2,3-Dioxygenase (TD02) gene; TCP-1, Mc4R and CART genes; interleukin-1 beta, tumor necrosis factor-alpha, intracellular adhesion molecule, and interleukin-8 and 10 genes; interferon-alpha gene; Ras-Protein and (HLA-DRB10404 and 0101or PTPN22 R620W); Dopamine Receptor D3 Ser9Gly (−205-G/A, −7685-G/C); Glutamine:fructose-6-phosphate amidotransferase (GFPT1 or GFPT 2) variant in exon 14, I471V or 3′ UTR, or glucosamine 6-P acyltransferase; Aggrecan proteoglycan allele 27; 11-beta hydroxysteroid dehydrogenase typel; FK506 binding protein 5; serum/glucosteroid kinase; Human tryptophan 2,3 dioxygenase; Myelin and Myelin associated glycoprotein genes (myelin oligodendrocyte glycoprotein (MOG), a tetranucleotide TAAA repeat (MOG4), C10991T SNP); Edg2; Fgfr2; Decorin; Brevican; Neurotensin (NT) receptors-1; Neurotensin (NT) receptors-2; Neurotensin (NT) receptors-3; Proenkephalin; prodynorphin(946C>G); Bdnf (Neurotrophic Factor (BDNF) Val66Met and-281 C>A, T allele of the C270T); Sgk(Serum-and glucose-regulated kinase (SGK 1) SNP Intron 6, Exon 8 (CC, CT, TT); Gab1; Id2; COMT; ANKK1; DAT1; DBH; HTT; HTR1A; HTR1D; HTR2A; HTR2C (5-HT-2A, 5-HT 2B, 5-HT-4 & 5-HT-7); ADRA2A; ADRA2; NET; MAOA; GABRA3; GABRB3; CNR1; CNRA4; NMDAR1; POMC; MGPAT; NYP; AgRP; OBR; Mc3R:UCP-1; GLUT4; PDGS; ALdB; LNC2; E23K Kir6.2 polymorphism; steroid sulfatase (STS) gene variation; G82G at the PTPN1 IVS6+G82A polymorphism; Sulfonylurea receptor 1; beta(3)-AR Trp64Arg; PC1; GHRELIN gene polymorphisms; FKBP5; VITAMIN D RECEPTOR GENE POLYMORPHISMS (BSMI AND FOKI; The lymphoid tyrosine phosphates (LYP), encoded by the protein tyrosine phosphatase-22 (PTPN22) gene, and all sodium ATPAses.

50. The method of claim 13, wherein the DNA collected is analyzed for variation of the CT and TT allele of the dopamine D2 receptor gene for indentifying a differential response to the nicotine patch.

51. The method of claim 13, wherein the DNA collected is analyzed for polymorphisms in the proinflammatory cytokine tumor necrosis factor (TNF) for identifying a differential response to fish oil supplementation for the treatment of rheumatoid arthritis.

52. The method of claim 13, wherein the DNA collected is analyzed for polymorphisms in the TNF gene for identifying a differential response to vitamin E for promoting anti-oxidant activity and reducing inflammatory processes.

53. The method of claim 13, wherein the DNA collected is analyzed for polymorphisms in the carbohydrate responsive element-binding protein (ChREBP), a key regulator of glucose metabolism and fat storage, cyclic AMP and a high fat diet inhibit ChREBP and slow down glucose utilization and based on the DNA result we will adjust the nutrient chromium (all salts), Banaba extract and Gymnea Sylvestre.

54. The method of claim 13, wherein the DNA collected is analyzed for polymorphisms of the dopamine D2 receptor gene for identifying a differential response to chromium salts.

55. The method of claim 13, 34 or 35, wherein the DNA collected is analyzed for at least one of the following polymorphisms of the dopamine D2, D1, D3, D4, and D5 receptor gene and information obtained is utilized to adjust the dosage of Synaptamine complex for pain control.

56. The method of claim 13, wherein the DNA collected is analyzed for polymorphisms of the human chromosome 2, uncoupling protein 2 and the APO-E genes, ob gene, PC1 and GHRELIN gene polymorphisms and information obtained is utilized to adjust the dosage of desnutrin.

57. The method of claim 13, wherein the DNA collected is analyzed for polymorphisms of PPAR-gamma 2; c-fos; c-jun and c-myc genes and information obtained is utilized to adjust the dosage of Banaba extract and/or Tannic acid.

58. The method of claim 13, wherein the DNA collected is analyzed for polymorphisms of Sterol Regulatory Element Protein-1 (SREBP-1c); mitochondrial glycerol-3-phosphate acyltransferase gene (MGPAT) and the peroxisome proloferator-activated receptor (PPAR-gamma-2) and information obtained is utilized to adjust the dosage desnutrin and adiponitrin.

59. The method of claim 13, wherein the DNA collected is analyzed for polymorphisms of the human TD02 gene and information obtained is utilized to adjust the dosage of L-tyrptophan, 5-hydroxytryptophan and chromium salts.

60. The method of claim 13, wherein the DNA collected is analyzed for polymorphisms of polymorphisms of TCP-1, Mc4R and CART and information obtained is utilized to adjust the dosage of appetite suppressants such as hoodia gordonii extracts, ephedrine, amphetamines, Synapatamine complex, synephrine, and citrus aurantium.

61. The method of claim 13, wherein the DNA collected is analyzed for polymorphisms of polymorphisms of the interleukin-1 alpha, interleukin 1 beta, tumor necrosis factor-alpha, intracellular adhesion molecule, interleukin-8, and interleukin-10 genes and information obtained is utilized to adjust the dosage of Echinacea.

62. The method of claim 13, wherein the DNA collected is analyzed for polymorphisms of polymorphisms of the Ras-Protein and HLA-DRB1 *0404 and *0101or PTPN22 R620W genes and interleukin-10 genes and information obtained is utilized to adjust the dosage of Ganoderma Lucidum.

63. The method of claim 13, wherein the DNA collected is analyzed for for polymorphisms of Dopamine Receptor D3 Ser9Gly (−205-G/A, −7685-G/C) gene and information obtained is utilized to adjust the dosage of Gamma-Mangostin.

64. The method of claim 13, wherein the DNA collected is analyzed for polymorphisms of Glutamine:fructose-6-phosphate amidotransferase (GFPT1 or GFPT 2) variant in exon 14, 1471V or 3′ UTR, or glucosamine 6-P ascetyltransferase genes and information obtained is utilized to adjust the dosage of Glucosamine Sulfate.

65. The method of claim 13, wherein the DNA collected is analyzed for polymorphisms of Aggrecan proteoglycan allele 27 gene and information obtained is utilized to adjust the dosage of Chondroitin Sulfate.

66. The method of claim 13, wherein the DNA collected is analyzed for polymorphisms of MTHFR C677T (heterozygous/homozygous mutant versus homozygous normal) gene and information obtained is utilized to adjust the dosage of Folic Acid.

67. The method of claim 13, wherein the DNA collected is analyzed for polymorphisms of collected will be analyzed for polymorphisms of hippocalcin like 1 (Hpcall) gene and information obtained is utilized to adjust the dosage of calcium.

68. The method of claim 13, wherein the DNA collected is analyzed for polymorphisms of proenkephalin, prodynorphin, neurotensin (1,2,3) Bdnf, TD02, Sgk, Fkbp5&4, Edg2, Id2, Gab1 Fgfr2 genes and information obtained is utilized to adjust the dosage of Passion flower.

69. The method of claim 13, wherein the DNA collected is analyzed for polymorphisms of proenkephalin, prodynorphin, neurotensin (1,2,3) Bdnf, TD02, Sgk, Fkbp5&4, Edg2, Id2, Gab1 Fgfr2 genes and information obtained is utilized to adjust the dosage of Kava Kava.

70. The method of claim 13, wherein the DNA collected is analyzed for polymorphisms of COMT, proenkephalin, prodynorphin, neurotensin (1,2,3) Bdnf, TD02, Sgk, Fkbp5&4, Edg2, Id2, Gab1 Fgfr2 genes and information obtained is utilized to adjust the dosage of Rhodiola rosea.

71. The method of claim 13, wherein the DNA collected is analyzed for polymorphisms of COMT, proenkephalin, prodynorphin, neurotensin (1,2,3) Bdnf, TD02, Sgk, Fkbp5&4, Edg2, Id2 genes and information obtained is utilized to adjust the dosage of Rhodendron.

72. The method of claim 13, wherein the DNA collected is analyzed for polymorphisms of COMT, DRD1-5, ANKK1, DAT1, DBH, TD02, HTT, HTR1A, HTR1D, HTR2A, HTR2C, ADRA2A, ADRA2, NET, MAOA, GABRA3, GABRB3, CNR1, CNRA4, NMDAR1, POMC genes and information obtained is utilized to adjust the dosage of dl-phenylalanine.

73. The method of claim 13, wherein the DNA collected is analyzed for polymorphisms of COMT NET MAOA DRD1-5 ANKK1 DAT1 DBH POMC proenkephalin, prodynorphin, neurotensin (1,2,3) Bdnf, TD02, Sgk, Fkbp5&4, Edg2, Id2, Gab1 Fgfr2 genes and information obtained is utilized to adjust the dosage of L-Tyrosine.

74. The method of claim 13, wherein the DNA collected is analyzed for polymorphisms of COMT NET MAOA POMC Proenkephalin, prodynorphin, neurotensin (1,2,3) GABRA3 GABRB3 NMDAR1 genes and information obtained is utilized to adjust the dosage of L-glutamine.

75. The method of claim 13, wherein the DNA collected is analyzed for polymorphisms of COMT NET MAOA POMC proenkephalin, prodynorphin, neurotensin (1,2,3) TD02, HTT, HTR1A, HTR1D, HTR2A, HTR2C genes and information obtained is utilized to adjust the dosage of 5-Hyroxytryptophane.

76. The method of claim 13, wherein the DNA collected is analyzed for polymorphisms of COMT, NET, MAOA, POMC, proenkephalin, prodynorphin, neurotensin (1,2,3), TD02, HTT, HTR1A, HTR1D, HTR2A, HTR2C, DRD1-5, ANKK1 HTR2A, HTR2C, DRD1-5, ANKK1, DAT1, DBH. genes and information obtained is utilized to adjust the dosage of Chromium (all salts).

77. The method of claim 13, wherein the DNA collected is analyzed for polymorphisms of HTT; HTR1A; HTR1D; HTR2A; HTR2C (5-HT-2A, 5-HT 2B, 5-HT-4 & 5-HT-7), COMT, D ANKK1, DAT1, DBH, TD02, ADRA2A, ADRA2, NET, MAOA, GABRA3, GABRB3, CNR1, CNRA4, NMDAR1, POMC, proenkephalin, prodynorphin, neurotensin (1,2,3), Bdnf, TD02, Sgk, Fkbp5&4, Edg2, Id2, Gab1, Fgfr2 genes and information obtained is utilized to adjust the dosage of (−)-Hydroxycitric acid.(HCA).

78. The method of claim 13, wherein the DNA collected is analyzed for polymorphisms of Hpcall COMT NET MAOA genes and information obtained is utilized to adjust the dosage of Pyridoxyl phosphate.

79. The method of claim 13, wherein the DNA collected is analyzed for polymorphisms of Hpcall gene and all ATPAse genes and information obtained is utilized to adjust the dosage of Magnesium.

80. The method of claim 13, wherein the DNA collected is analyzed for polymorphisms of leptin receptor, dopamineD1-5, Hpcall, HTT, HTR1A, HTR1D, HTR2A, HTR2C (5-HT-2A, 5-HT 2B, 5-HT-4 & 5-HT-7), ANKK1, DAT1, DBH, TD02 and information obtained is utilized to adjust the dosage of potassium.

81. The method of claim 13, wherein the DNA collected is analyzed for polymorphisms of interferon-CD8A, or PS1, SREBP-1c, PPAR-gamma-2, MGPAT. NYP, AgRP, POMC, CART, OBR, Mc3R, Mc4R, UCP-1, GLUT4, C-FOS, C-JUN, C-MYC, Interleukin 1-alpha, interleukin-1 beta, interleukin-8, tumor necrosis factor-alpha, intracellular adhesion molecule, interleikin-10, genes and information obtained is utilized to adjust the dosage of Magnolia.

82. The method of claim 13, wherein the composition administered comprises at least one of the following glyconutrients: Glucose, fucose, mannose, galactose, xylose, N-acetyl-glucosamine, N-acetyl-galactosamine, N-acetyl-neuraminic acid (sialic acid), arabinose, arabinogalactans, and DL-phenylalanine.

83. The method of claim 82, wherein the glyconutrients are comprised in at least one of the following foodstuffs: aloe, fenugreek, numerous species of medicinal mushrooms, Wester Larch (tree sap and bark) and other glycoside rich botanical substances.

84. The method of claim 34, wherein the composition administered further comprises at least one of the following: (−)-Hydroxycitric acid (HCA); Passion flower (Passionflora incarata L Extract; Potassium; Thiamin; Vitamin B₅; and Calcium in a daily therapeutic amount ranging from approximately 1 mcg to 30,000 mg.

85. The method of claim 13, wherein the composition administered comprises at least one immunomodulator wherein the immunomodulator comprises 2-3000 mg Protykin for the treatment of at least one of the following disease states: Neurogenobolic Deficiency Syndrome (NGDS); Reward Deficiency Syndrome.(RDS); Joint Health (JH); all types of Diabetes; stress; and anxiety.

86. The composition of claim 34, 35, 36, 37, or 38, wherein the composition is administered for the treatment of at least one of the following disease states: Neurogenobolic Deficiency Syndrome (NGDS); Reward Deficiency Syndrome.(RDS); Joint Health (JH); all types of Diabetes; stress; and anxiety.
